(19) United States
(12) Patent Application Publication
(10) Pub. No.: US 2020/0246359 A1
(43) Pub. Date: Aug. 6, 2020

(54) COMPOSITIONS AND METHODS FOR TREATING OPHTHALMIC CONDITIONS

(71) Applicant: MEDICON PHARMACEUTICALS, INC., Setauket, NY (US)

(72) Inventor: Basil RIGAS, Setauket, NY (US)

(73) Assignee: MEDICON PHARMACEUTICALS, INC., Setauket, NY (US)

(21) Appl. No.: 16/651,846

(22) PCT Filed: Sep. 28, 2018
(86) PCT No.: PCT/US2018/053451

§ 371 Date: Mar. 27, 2020

Related U.S. Application Data
(60) Provisional application No. 62/564,595, filed on Sep. 28, 2017.
(60) Provisional application No. 62/649,273, filed on Mar. 28, 2018.

(51) Int. Cl. A61K 31/661; A61K 47/40; A61K 47/22; A61K 47/26; A61K 47/32; A61K 47/10; A61P 27/02; A61K 47/18

(52) U.S. Cl. CPC A61K 31/661; A61K 47/40; A61K 47/22; A61K 47/26; A61K 45/06; A61K 47/10; A61P 27/02; A61K 47/18; A61K 47/32

(57) ABSTRACT

Methods and compositions that include the use NSAID derivatives for the treatment of disease, retinopathy, and related diseases are disclosed herein.

### CROSS-REFERENCE TO RELATED APPLICATIONS

This application claims the benefit of U.S. Provisional Patent Application No. 62/564,595, filed on Sep. 28, 2017, and U.S. Provisional Patent Application No. 62/649,273, filed Mar. 28, 2018, which are hereby incorporated by reference in their entirety.

### FIELD OF THE INVENTION

The invention relates generally to compounds and methods of using the same for treating conditions of the eye and more particularly, but not exclusively, to the use of phosphosulindac for the treatment of dry eye, retinopathy, and related disorders.

### BACKGROUND OF THE INVENTION

The eye consists of the eyeball and its adnexa, which includes the structures outside of the eyeball, such as the orbit, eye muscles, eyelids, eyelashes, conjunctiva, and lacrimal apparatus. The eye and its various structures may be affected by a number of pathological conditions including various inflammatory, autoimmune, and metabolic conditions.

### SUMMARY OF THE INVENTION

In order to address the needs in the field, the invention includes compounds, compositions, and methods for treating various conditions of the eye and its associated structures (i.e., ophthalmic conditions). In some embodiments, the ophthalmic conditions treated by the compounds, compositions, and/or kits may include dry eye disease and retinopathy. In some embodiments, retinopathy may include the diseases of diabetic retinopathy, retinopathy of prematurity, VEGF retinopathy, age related macular degeneration, retinal vein occlusion, and/or hypertensive retinopathy. In certain embodiments, retinopathy may be diabetic retinopathy.

In some embodiments, the invention may include compositions, methods, or kits that comprise or use an NSAID derivative as described herein. In some embodiments, the NSAID derivative may be a compound of formula I or formula II:

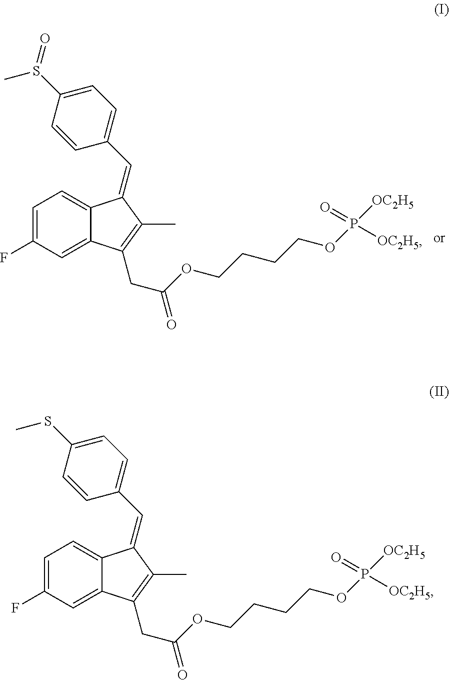 [SMILES: CCOP(=O)(OCC)OCCCCOC(=O)CC1=C(C)/C(=C/c2ccc(S(C)=O)cc2)c2ccc(F)cc21 ; CCOP(=O)(OCC)OCCCCOC(=O)CC1=C(C)/C(=C/c2ccc(SC)cc2)c2ccc(F)cc21]

or a pharmaceutically acceptable salt thereof. The compound of formula I may be referred to as phosphosulindac (PS). Any compositions and formulation described herein as including PS, can include either PS, PS-II, or both. The compound of formula II may be referred to as phosphosulindac II (PS-II). The compounds of formulas I and II are described in U.S. Pat. No. 8,236,820, the entirety of which is incorporated herein by reference.

In an embodiment, the invention includes a composition for the treatment of dry eye disease comprising a therapeutically effective amount of a compound of formula I or formula II, or a pharmaceutically acceptable salt thereof, and a pharmaceutically acceptable carrier.

In one embodiment, the invention relates to a composition for treating an ophthalmic condition in a patient in need thereof, wherein the ophthalmic condition is selected from the group consisting of dry eye disease and retinopathy, the composition comprising an emulsion comprising a therapeutically effective amount of a compound of formula I or formula II:

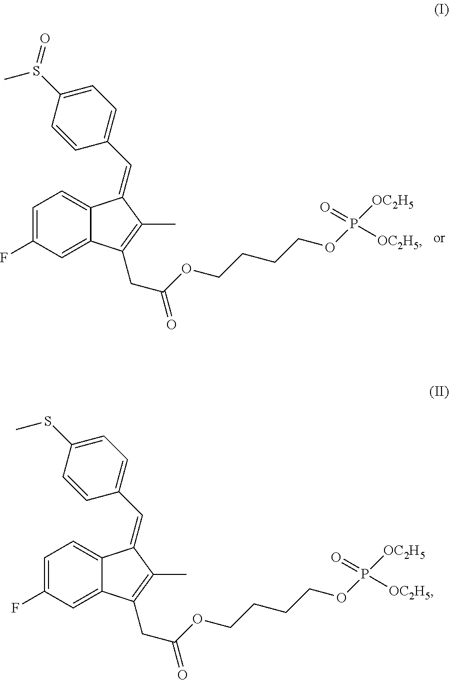 [SMILES: CCOP(=O)(OCC)OCCCCOC(=O)CC1=C(C)/C(=C/c2ccc(S(C)=O)cc2)c2ccc(F)cc21 ; CCOP(=O)(OCC)OCCCCOC(=O)CC1=C(C)/C(=C/c2ccc(SC)cc2)c2ccc(F)cc21]

or a pharmaceutically acceptable salt thereof, and a pharmaceutically acceptable carrier. In some embodiments, the emulsion comprises a therapeutically effective amount of a compound of formula I, or a pharmaceutically acceptable salt thereof. In some embodiments, the ophthalmic condition is dry eye disease. In some embodiments, the ophthalmic condition is retinopathy, which is selected from the group consisting of diabetic retinopathy, retinopathy of prematurity, VEGF retinopathy, age related macular degeneration, retinal vein occlusion, and hypertensive retinopathy. In some embodiments, the ophthalmic condition is diabetic retinopathy. In some embodiments, the emulsion comprises between about 0.01% and about 10% of a compound of formula I or formula II. In some embodiments, the emulsion further comprises between about 0.01% and about 10% propylene glycol. In some embodiments, the emulsion further comprises between about 1% and about 25% mineral oil. In some embodiments, the emulsion further comprises between about 0.5% and about 10% of one or more of Tween 60 and Tween 80. In some embodiments, the emulsion further comprises between about 1% and about 25% of (2-hydroxypropyl)-β-cyclodextrin (HP-(3-CD).

In one embodiment, the invention relates to a composition for treating an ophthalmic condition in a patient in need thereof, wherein the ophthalmic condition is selected from the group consisting of dry eye disease and retinopathy, the composition comprising an emulsion comprising between about 0.01% and about 10% of a compound of formula I or formula II; between about 0.01% and about 10% propylene glycol; between about 1% and about 25% mineral oil; between about 0.5% and about 10% of one or more of Tween 60 and Tween 80; and between about 1% and about 25% of (2-hydroxypropyl)-β-cyclodextrin (HP-(3-CD).

In one embodiment, the invention relates to a composition for treating an ophthalmic condition in a patient in need thereof, wherein the ophthalmic condition is selected from the group consisting of dry eye disease and retinopathy, the composition comprising an emulsion comprising about 0.5%, about 1%, about 1.5%, about 2%, about 2.5%, about 3%, about 3.5%, about 4%, about 4.5%, or about 5% of a compound of formula I or formula II; between about 0.01% and about 10% propylene glycol; between about 1% and about 25% mineral oil; between about 0.5% and about 10% of one or more of Tween 60 and Tween 80; and between about 1% and about 25% of (2-hydroxypropyl)-β-cyclodextrin (HP-β-CD).

In one embodiment, the invention relates to a composition for treating an ophthalmic condition in a patient in need thereof, wherein the ophthalmic condition is selected from the group consisting of dry eye disease and retinopathy, the composition comprising an emulsion comprising between about 0.01% and about 10% of a compound of formula I or formula II; about 1%, about 2%, about 3%, about 4%, about 5%, about 6%, about 7%, about 8%, about 9%, or about 10% propylene glycol; between about 1% and about 25% mineral oil; between about 0.5% and about 10% of one or more of Tween 60 and Tween 80; and between about 1% and about 25% of (2-hydroxypropyl)-β-cyclodextrin (HP-β-CD).

In one embodiment, the invention relates to a composition for treating an ophthalmic condition in a patient in need thereof, wherein the ophthalmic condition is selected from the group consisting of dry eye disease and retinopathy, the composition comprising an emulsion comprising between about 0.01% and about 10% of a compound of formula I or formula II; between about 0.01% and about 10% propylene glycol; about 5%, about 6%, about 7%, about 8%, about 9%, about 10%, about 11%, about 12%, about 13%, about 14%, or about 15% mineral oil; between about 0.5% and about 10% of one or more of Tween 60 and Tween 80; and between about 1% and about 25% of (2-hydroxypropyl)-β-cyclodextrin (HP-β-CD).

In one embodiment, the invention relates to a composition for treating an ophthalmic condition in a patient in need thereof, wherein the ophthalmic condition is selected from the group consisting of dry eye disease and retinopathy, the composition comprising an emulsion comprising between about 0.01% and about 10% of a compound of formula I or formula II; between about 0.01% and about 10% propylene glycol; between about 1% and about 25% mineral oil; about 1%, about 2%, about 3%, about 4%, about 5%, about 6%, about 7%, about 8%, about 9%, or about 10% Tween 60; and between about 1% and about 25% of (2-hydroxypropyl)-β-cyclodextrin (HP-β-CD).

In one embodiment, the invention relates to a composition for treating an ophthalmic condition in a patient in need thereof, wherein the ophthalmic condition is selected from the group consisting of dry eye disease and retinopathy, the composition comprising an emulsion comprising between about 0.01% and about 10% of a compound of formula I or formula II; between about 0.01% and about 10% propylene glycol; between about 1% and about 25% mineral oil; about 1%, about 2%, about 3%, about 4%, about 5%, about 6%, about 7%, about 8%, about 9%, or about 10% Tween 80; and between about 1% and about 25% of (2-hydroxypropyl)-β-cyclodextrin (HP-β-CD).

In one embodiment, the invention relates to a composition for treating an ophthalmic condition in a patient in need thereof, wherein the ophthalmic condition is selected from the group consisting of dry eye disease and retinopathy, the composition comprising an emulsion comprising between about 0.01% and about 10% of a compound of formula I or formula II; between about 0.01% and about 10% propylene glycol; between about 1% and about 25% mineral oil; about 1%, about 2%, about 3%, about 4%, about 5%, about 6%, about 7%, about 8%, about 9%, or about 10% Tween 60; about 1%, about 2%, about 3%, about 4%, about 5%, about 6%, about 7%, about 8%, about 9%, or about 10% Tween 80; and between about 1% and about 25% of (2-hydroxypropyl)-β-cyclodextrin (HP-β-CD).

In one embodiment, the invention relates to a composition for treating an ophthalmic condition in a patient in need thereof, wherein the ophthalmic condition is selected from the group consisting of dry eye disease and retinopathy, the composition comprising an emulsion comprising between about 0.01% and about 10% of a compound of formula I or formula II; between about 0.01% and about 10% propylene glycol; between about 1% and about 25% mineral oil; between about 0.5% and about 10% of one or more of Tween 60 and Tween 80; and about 5%, about 6%, about 7%, about 8%, about 9%, about 10%, about 11%, about 12%, about 13%, about 14%, or about 15% (2-hydroxypropyl)-β-cyclodextrin (HP-β-CD).

In one embodiment, the invention relates to a composition for treating an ophthalmic condition in a patient in need thereof, wherein the ophthalmic condition is selected from the group consisting of dry eye disease and retinopathy, the composition comprising an emulsion comprising about 2% of a compound of formula I or formula II; about 5% propylene glycol; about 10% mineral oil; about 4% Tween 60; about 4% Tween 80; and about 10% (2-hydroxypropyl)-β-cyclodextrin (HP-β-CD).

In an embodiment, the invention includes a composition for the treatment of dry eye disease comprising a therapeutically effective amount of a compound of formula I or formula II, or a pharmaceutically acceptable salt thereof, and a therapeutically effective amount of an additional active agent, and a pharmaceutically acceptable carrier. In some embodiments, the additional active agent may include one or more of an antibiotic, cyclosporine, and lifitegrast.

In some embodiments, the invention includes a composition for the treatment of dry eye disease comprising a therapeutically effective amount of a compound of formula I, or a pharmaceutically acceptable salt thereof, and a pharmaceutically acceptable carrier.

In an embodiment, the invention includes a method for treating dry eye disease in a patient in need thereof, the method comprising administering to the patient a therapeutically effective amount of a compound of formula I or formula II, or a pharmaceutically acceptable salt thereof.

In an embodiment, the invention includes a method for treating dry eye disease in a patient in need thereof, the method comprising administering to the patient a therapeutically effective amount of a compound of formula I or formula II, or a pharmaceutically acceptable salt thereof, and a therapeutically effective amount of an additional active agent. In some embodiments, the additional active agent may include one or more of an antibiotic, cyclosporine, and lifitegrast.

In some embodiments, the invention includes a method for treating dry eye disease in a patient in need thereof, the method comprising administering to the patient a therapeutically effective amount of a compound of formula I, or a pharmaceutically acceptable salt thereof.

In an embodiment, the invention includes a composition for the treatment of retinopathy comprising a therapeutically effective amount of a compound of formula I or formula II, or a pharmaceutically acceptable salt thereof, and a pharmaceutically acceptable carrier.

In an embodiment, the invention includes a composition for the treatment of retinopathy comprising a therapeutically effective amount of a compound of formula I or formula II, or a pharmaceutically acceptable salt thereof, and a therapeutically effective amount of an additional active agent, and a pharmaceutically acceptable carrier. In some embodiments, the additional active agent may include one or more of an antibiotic, cyclosporine, and lifitegrast.

In some embodiments, the antibiotic may include one or more of tetracycline, tobramycin, chlortetracycline, bacitracin, neomycin, polymyxin, gramicidin, oxytetracycline, chloramphenicol, gentamycin, and erythromycin. Other antibiotics include aminoglycoside, ampicillin, carbenicillin, cefazolin, cephalosporin, chloramphenicol, clindamycin, everninomycin, gentamycin, kanamycin, lipopeptides, methicillin, nafcillin, novobiocia, oxazolidinones, penicillin, quinolones, rifampin, streptogramins, streptomycin, sulfamethoxazole, sulfonamide, trimethoprim, and vancomycin.

In some embodiments, the invention includes a composition for the treatment of retinopathy comprising a therapeutically effective amount of a compound of formula I, or a pharmaceutically acceptable salt thereof, and a pharmaceutically acceptable carrier.

In an embodiment, the invention includes a method for treating retinopathy in a patient in need thereof, the method comprising administering to the patient a therapeutically effective amount of a compound of formula I or formula II, or a pharmaceutically acceptable salt thereof.

In an embodiment, the invention includes a method for treating retinopathy in a patient in need thereof, the method comprising administering to the patient a therapeutically effective amount of a compound of formula I or formula II, or a pharmaceutically acceptable salt thereof, and a therapeutically effective amount of an additional active agent. In some embodiments, the additional active agent may include one or more of an antibiotic, cyclosporine, and lifitegrast.

In some embodiments, the invention includes a method for treating retinopathy in a patient in need thereof, the method comprising administering to the patient a therapeutically effective amount of a compound of formula I, or a pharmaceutically acceptable salt thereof.

In an embodiment, the invention includes a method of treating an ophthalmic condition in a patient in need thereof, wherein the ophthalmic condition is selected from the group consisting of dry eye disease and retinopathy, the method comprising administering to the patient a therapeutically effective amount of a compound with reduced risk of corneal melt of formula I or formula II, or a pharmaceutically acceptable salt thereof.

In an embodiment, the invention includes a composition for treating an ophthalmic condition in a patient in need thereof, wherein the ophthalmic condition is selected from the group consisting of dry eye disease and retinopathy, the composition comprising a therapeutically effective amount of a compound with reduced risk of corneal melt of formula I or formula II, or a pharmaceutically acceptable salt thereof, and a pharmaceutically acceptable carrier.

In an embodiment, the invention includes a composition for treating an ophthalmic condition in a patient in need thereof, wherein the ophthalmic condition is selected from the consisting of dry eye disease and retinopathy, the group composition comprising a therapeutically effective amount of a compound of formula I or formula II, or a pharmaceutically acceptable salt thereof, a pharmaceutically acceptable carrier, and one or more of a solubilizing agent (e.g., vitamin E TPGS (d-α-tocopheryl polyethylene glycol 1000 succinate)), a sugar alcohol (e.g., mannitol), an acid (e.g., boric acid), and a preservative (e.g., polyquaternium-1 (polyquad)). In some embodiments, such formulations may be used to deliver a compound of formula I or formula II, or a pharmaceutically acceptable salt thereof, to the retina following topical administration to the eye.

In an embodiment, the invention includes a composition for treating an ophthalmic condition in a patient in need thereof, wherein the ophthalmic condition is selected from the group consisting of dry eye disease and retinopathy, the composition comprising, by weight, about 0.5% to about 10% of a compound of formula I or formula II, or a pharmaceutically acceptable salt thereof, a pharmaceutically acceptable carrier, and one or more of about 0% to about 25% vitamin E TPGS (d-α-tocopheryl polyethylene glycol 1000 succinate), about 0% to about 10% mannitol, about 0% to about 10% boric acid, and about 0% to about 1% polyquaternium-1 (polyquad).

In an embodiment, the invention includes a composition for treating an ophthalmic condition in a patient in need thereof, wherein the ophthalmic condition is selected from the group consisting of dry eye disease and retinopathy, the composition comprising, by weight, greater than 0.5% of a compound of formula I or formula II, or a pharmaceutically acceptable salt thereof, a pharmaceutically acceptable carrier, and one or more of greater than 5% vitamin E TPGS (d-α-tocopheryl polyethylene glycol 1000 succinate), greater than 0.5% mannitol, greater than 0.5% boric acid, and greater than 0.001% polyquaternium-1 (polyquad).

In an embodiment, the invention includes a composition for treating an ophthalmic condition in a patient in need thereof, wherein the ophthalmic condition is selected from the group consisting of dry eye disease and retinopathy, the composition comprising, by weight, less than 10% of a compound of formula I or formula II, or a pharmaceutically acceptable salt thereof, a pharmaceutically acceptable carrier, and one or more of less than 25% vitamin E TPGS (d-α-tocopheryl polyethylene glycol 1000 succinate), less than 10% mannitol, less than 10% boric acid, and less than 1% polyquaternium-1 (polyquad).

In an embodiment, the invention includes a composition for treating an ophthalmic condition in a patient in need thereof, wherein the ophthalmic condition is selected from the group consisting of dry eye disease and retinopathy, the composition comprising, by weight, about 3.5% of a compound of formula I or formula II, or a pharmaceutically acceptable salt thereof, a pharmaceutically acceptable carrier, and one or more of about 16% vitamin E TPGS (d-α-tocopheryl polyethylene glycol 1000 succinate), about 3.18% mannitol, about 1.2% boric acid, and about 0.005% polyquaternium-1 (polyquad).

In an embodiment, the invention includes a composition for treating an ophthalmic condition in a patient in need thereof, wherein the ophthalmic condition is selected from the group consisting of dry eye disease and retinopathy, the composition comprising a therapeutically effective amount of a compound of formula I or formula II, or a pharmaceutically acceptable salt thereof, a pharmaceutically acceptable carrier, and one or more of a gelling excipient (e.g., gellan gum or sodium alginate), a poloxamer, a solubilizing agent (e.g., vitamin E TPGS), a surfactant, a polyether, and a cyclodextrin (e.g., (2-hydroxypropyl)-β-cyclodextrin). In some embodiments, such formulations may allow for delivery of a compound of formula I or formula II, or a pharmaceutically acceptable salt thereof, to anterior segments of the eye following topical administration.

In an embodiment, the invention includes a composition for treating an ophthalmic condition in a patient in need thereof, wherein the ophthalmic condition is selected from the group consisting of dry eye disease and retinopathy, the composition comprising a therapeutically effective amount of a compound of formula I or formula II, or a pharmaceutically acceptable salt thereof, a pharmaceutically acceptable carrier, and one or more of gellan gum, vitamin E TPGS, and a (2-hydroxypropyl)-β-cyclodextrin.

In an embodiment, the invention includes a composition for treating an ophthalmic condition in a patient in need thereof, wherein the ophthalmic condition is selected from the group consisting of dry eye disease and retinopathy, the composition comprising, by weight, about 0.5% to about 10% of a compound of formula I or formula II, or a pharmaceutically acceptable salt thereof, a pharmaceutically acceptable carrier, and one or more of about 0% to about 5% gellan gum, about 0% to about 20% vitamin E TPGS, and about 0% to about 20% (2-hydroxypropyl)-β-cyclodextrin.

In an embodiment, the invention includes a composition for treating an ophthalmic condition in a patient in need thereof, wherein the ophthalmic condition is selected from the group consisting of dry eye disease and retinopathy, the composition comprising, by weight, greater than 0.5% of a compound of formula I or formula II, or a pharmaceutically acceptable salt thereof, a pharmaceutically acceptable carrier, and one or more of greater than 0.1% gellan gum, greater than 1% vitamin E TPGS, and greater than 5% (2-hydroxypropyl)-β-cyclodextrin.

In an embodiment, the invention includes a composition for treating an ophthalmic condition in a patient in need thereof, wherein the ophthalmic condition is selected from the group consisting of dry eye disease and retinopathy, the composition comprising, by weight, less than 20% of a compound of formula I or formula II, or a pharmaceutically acceptable salt thereof, a pharmaceutically acceptable carrier, and one or more of less than 5% gellan gum, less than 20% vitamin E TPGS, less than 20% (2-hydroxypropyl)-β-cyclodextrin.

In an embodiment, the invention includes a composition for treating an ophthalmic condition in a patient in need thereof, wherein the ophthalmic condition is selected from the group consisting of dry eye disease and retinopathy, the composition comprising, by weight, about 2.4% to about 3% of a compound of formula I or formula II, or a pharmaceutically acceptable salt thereof, a pharmaceutically acceptable carrier, and one or more of about 0.5% gellan gum, about 5% vitamin E TPGS, about 10% (2-hydroxypropyl)-β-cyclodextrin.

In an embodiment, the invention includes a composition for treating an ophthalmic condition in a patient in need thereof, wherein the ophthalmic condition is selected from the group consisting of dry eye disease and retinopathy, the composition comprising, by weight, about 2.4% to about 3% of a compound of formula I or formula II, or a pharmaceutically acceptable salt thereof, a pharmaceutically acceptable carrier, and one or more of about 0.4% gellan gum, about 10% vitamin E TPGS, about 5% (2-hydroxypropyl)-β-cyclodextrin.

In an embodiment, the invention includes a composition for treating an ophthalmic condition in a patient in need thereof, wherein the ophthalmic condition is selected from the group consisting of dry eye disease and retinopathy, the composition comprising a therapeutically effective amount of a compound of formula I or formula II, or a pharmaceutically acceptable salt thereof, a pharmaceutically acceptable carrier, and one or more of sodium alginate, vitamin E TPGS, a (2-hydroxypropyl)-β-cyclodextrin, Tween (e.g., Tween 80), poly(ethylene glycol) (PEG) (e.g., PEG 400), and polyoxyl stearate.

In an embodiment, the invention includes a composition for treating an ophthalmic condition in a patient in need thereof, wherein the ophthalmic condition is selected from the group consisting of dry eye disease and retinopathy, the composition comprising, by weight, about 0.5% to about 10% of a compound of formula I or formula II, or a pharmaceutically acceptable salt thereof, a pharmaceutically acceptable carrier, and one or more of about 0% to about 5% sodium alginate, about 0% to about 20% vitamin E TPGS, and about 0% to about 20% (2-hydroxypropyl)-β-cyclodextrin.

In an embodiment, the invention includes a composition for treating an ophthalmic condition in a patient in need thereof, wherein the ophthalmic condition is selected from the group consisting of dry eye disease and retinopathy, the composition comprising, by weight, greater than 0.5% of a compound of formula I or formula II, or a pharmaceutically acceptable salt thereof, a pharmaceutically acceptable carrier, and one or more of greater than 0.1% sodium alginate, greater than 1% vitamin E TPGS, and greater than 5% (2-hydroxypropyl)-β-cyclodextrin.

In an embodiment, the invention includes a composition for treating an ophthalmic condition in a patient in need thereof, wherein the ophthalmic condition is selected from the group consisting of dry eye disease and retinopathy, the composition comprising, by weight, less than 10% of a compound of formula I or formula II, or a pharmaceutically acceptable salt thereof, a pharmaceutically acceptable carrier, and one or more of less than 5% sodium alginate, less than 20% vitamin E TPGS, less than 20% (2-hydroxypropyl)-β-cyclodextrin.

In an embodiment, the invention includes a composition for treating an ophthalmic condition in a patient in need thereof, wherein the ophthalmic condition is selected from the group consisting of dry eye disease and retinopathy, the composition comprising, by weight, about 3% of a compound of formula I or formula II, or a pharmaceutically acceptable salt thereof, a pharmaceutically acceptable carrier, and one or more of about 1.5% sodium alginate, about 5% vitamin E TPGS, about 10% (2-hydroxypropyl)-β-cyclodextrin.

In an embodiment, the invention includes a composition for treating an ophthalmic condition in a patient in need thereof, wherein the ophthalmic condition is selected from the group consisting of dry eye disease and retinopathy, the composition comprising, by weight, about 0.5% to about 10% of a compound of formula I or formula II, or a pharmaceutically acceptable salt thereof, a pharmaceutically acceptable carrier, and one or more of about 0% to about 5% sodium alginate, about 0% to about 25% Tween 80, about 0% to about 20% (2-hydroxylpropyl)-β-cyclodextrin, about 0% to about 20% PEG 400, and about 0% to about 10% polyoxyl stearate.

In an embodiment, the invention includes a composition for treating an ophthalmic condition in a patient in need thereof, wherein the ophthalmic condition is selected from the group consisting of dry eye disease and retinopathy, the composition comprising, by weight, greater than 0.5% of a compound of formula I or formula II, or a pharmaceutically acceptable salt thereof, a pharmaceutically acceptable carrier, and one or more of greater than 1% sodium alginate, greater than 1% Tween 80, greater than 1% (2-hydroxylpropyl)-β-cyclodextrin, greater than 1% PEG 400, and greater than 1% polyoxyl stearate.

In an embodiment, the invention includes a composition for treating an ophthalmic condition in a patient in need thereof, wherein the ophthalmic condition is selected from the group consisting of dry eye disease and retinopathy, the composition comprising, by weight, less than 10% of a compound of formula I or formula II, or a pharmaceutically acceptable salt thereof, a pharmaceutically acceptable carrier, and one or more of less than 5% sodium alginate, less than 25% Tween 80, less than 20% (2-hydroxylpropyl)-β-cyclodextrin, less than 20% PEG 400, and less than 10% polyoxyl stearate.

In an embodiment, the invention includes a composition for treating an ophthalmic condition in a patient in need thereof, wherein the ophthalmic condition is selected from the group consisting of dry eye disease and retinopathy, the composition comprising, by weight, 3% of a compound of formula I or formula II, or a pharmaceutically acceptable salt thereof, a pharmaceutically acceptable carrier, and one or more of about 1.5% sodium alginate, about 15% Tween 80, about 10% (2-hydroxylpropyl)-β-cyclodextrin, about 10% PEG 400, and about 5% polyoxyl stearate.

In an embodiment, the invention includes a composition for treating an ophthalmic condition in a patient in need thereof, wherein the ophthalmic condition is selected from the group consisting of dry eye disease and retinopathy, the composition comprising, by weight, about 1% to about 5% of a compound of formula I or formula II, or a pharmaceutically acceptable salt thereof, a pharmaceutically acceptable carrier, and one or more of about 50% to about 90% (2-hydroxypropyl)-β-cyclodextrin (HP-β-CD), about 0.05% to about 1% cremophor EL (F1), and about 0.5% to about 5% Tween 80 (F2).

In an embodiment, the invention includes a composition for treating an ophthalmic condition in a patient in need thereof, wherein the ophthalmic condition is selected from the group consisting of dry eye disease and retinopathy, the composition comprising, by weight, about 1% to about 5% of a compound of formula I or formula II, or a pharmaceutically acceptable salt thereof, a pharmaceutically acceptable carrier, and one or more of about 50% to about 90% (2-hydroxypropyl)-β-cyclodextrin (HP-β-CD), and about 0.05% to about 1% cremophor EL.

In an embodiment, the invention includes a composition for treating an ophthalmic condition in a patient in need thereof, wherein the ophthalmic condition is selected from the group consisting of dry eye disease and retinopathy, the composition comprising, by weight, about 1% to about 5% of a compound of formula I or formula II, or a pharmaceutically acceptable salt thereof, a pharmaceutically acceptable carrier, and one or more of about 50% to about 90% (2-hydroxypropyl)-β-cyclodextrin (HP-β-CD), and about 0.5% to about 5% Tween 80 (F2).

In an embodiment, the invention includes a composition for treating an ophthalmic condition in a patient in need thereof, wherein the ophthalmic condition is selected from the group consisting of dry eye disease and retinopathy, the composition comprising, by weight, about 3% to about 4% of a compound of formula I or formula II, or a pharmaceutically acceptable salt thereof, a pharmaceutically acceptable carrier, and one or more of about 80% (2-hydroxypropyl)-β-cyclodextrin (HP-β-CD), and about 0.1% cremophor EL.

In an embodiment, the invention includes a composition for treating an ophthalmic condition in a patient in need thereof, wherein the ophthalmic condition is selected from the group consisting of dry eye disease and retinopathy, the composition comprising, by weight, about 3% to about 4% of a compound of formula I or formula II, or a pharmaceutically acceptable salt thereof, a pharmaceutically acceptable carrier, and one or more of about 80% (2-hydroxypropyl)-β-cyclodextrin (HP-β-CD), and about 1% Tween 80 (F2).

In an embodiment, the invention includes a composition for treating an ophthalmic condition in a patient in need thereof, wherein the ophthalmic condition is selected from the group consisting of dry eye disease and retinopathy, the composition comprising, by weight, about 1% to about 10% of a compound of formula I or formula II, or a pharmaceutically acceptable salt thereof, a pharmaceutically acceptable carrier, and one or more of about 1% to about 40% Poloxamer 407 and about 1% to about 20% vitamin E TPGS.

In an embodiment, the invention includes a composition for treating an ophthalmic condition in a patient in need thereof, wherein the ophthalmic condition is selected from the group consisting of dry eye disease and retinopathy, the composition comprising, by weight, greater than 1% of a compound of formula I or formula II, or a pharmaceutically acceptable salt thereof, a pharmaceutically acceptable carrier, and one or more of greater than 1% Poloxamer 407 and greater than 1% vitamin E TPGS.

In an embodiment, the invention includes a composition for treating an ophthalmic condition in a patient in need thereof, wherein the ophthalmic condition is selected from the group consisting of dry eye disease and retinopathy, the composition comprising, by weight, less than 10% of a compound of formula I or formula II, or a pharmaceutically acceptable salt thereof, a pharmaceutically acceptable carrier, and one or more of less than 40% Poloxamer 407 and less than 20% vitamin E TPGS.

In an embodiment, the invention includes a composition for treating an ophthalmic condition in a patient in need thereof, wherein the ophthalmic condition is selected from the group consisting of dry eye disease and retinopathy, the composition comprising, by weight, about 5.4% of a compound of formula I or formula II, or a pharmaceutically acceptable salt thereof, a pharmaceutically acceptable carrier, and one or more of about 20% Poloxamer 407 and about 12% vitamin E TPGS.

In an embodiment, the invention includes a composition for treating an ophthalmic condition in a patient in need thereof, wherein the ophthalmic condition is selected from the group consisting of dry eye disease and retinopathy, the composition comprising a nanoparticle formulation comprising a compound of formula I or formula II, or a pharmaceutically acceptable salt thereof, and a pharmaceutically acceptable carrier. In some embodiments, the nanoparticle formulation may include poly(ethylene glycol) (PEG) nanoparticles. In some embodiments, the nanoparticle formulation may include methoxy poly(ethylene glycol)-poly(lactide) (mPEG-PLA) nanoparticles. In some embodiments, such formulations may allow for delivery of PS to anterior segments of the eye following topical administration.

In an embodiment, the invention includes a composition for treating an ophthalmic condition in a patient in need thereof, wherein the ophthalmic condition is selected from the group consisting of dry eye disease and retinopathy, the composition comprising a nanoparticle formulation comprising, by weight, about 1% to about 5% a compound of formula I or formula II, or a pharmaceutically acceptable salt thereof, a pharmaceutically acceptable carrier, and about 90% to about 98% mPEG-PLA.

In an embodiment, the invention includes a composition for treating an ophthalmic condition in a patient in need thereof, wherein the ophthalmic condition is selected from the group consisting of dry eye disease and retinopathy, the composition comprising a nanoparticle formulation comprising, by weight, about 3% to about 3.5% a compound of formula I or formula II, or a pharmaceutically acceptable salt thereof, a pharmaceutically acceptable carrier, and about 96.5% to about 97% mPEG-PLA.

In some embodiments, the compounds of formula I and/or formula II are analgesic agents.

In an embodiment, the invention includes an analgesic composition comprising about 0.1% to about 1% a compound of formula I or formula II, or a pharmaceutically acceptable salt thereof; about 10% to about 30% (2-hydroxypropyl)β-cyclodextrin (HP-β-CD); and about 0.1% to about 10% Tween 80.

In an embodiment, the invention includes an anesthetic composition comprising about 0.1% to about 1% a compound of formula I or formula II, or a pharmaceutically acceptable salt thereof; about 10% to about 30% (2-hydroxypropyl)β-cyclodextrin (HP-β-CD); and about 0.1% to about 10% Tween 80.

In some embodiments, the compounds of formula I and/or formula II are anti-inflammatory agents.

In some embodiments, the compounds of formula I and/or formula II have a reduced risk of corneal melt or do not result in corneal melt upon administration to the eye.

### BRIEF DESCRIPTION OF THE DRAWINGS

FIG. 1 illustrates the injection sites to the rabbit eye. The right eye of the rabbit and its two lacrimal glands are depicted along with the sites where Con A is administered. Part of the ILG is underneath the zygomatic bone. Upper right: orientation coordinates.

FIG. 2 illustrates ultrasonographic images of the head of the ILG before and after injection of Con A. The characteristic hypoechoic space seen in the post injection image confirms the success of the injection.

FIG. 3 illustrates that Con A induces inflammation in the lacrimal gland. Microtome sections of the head of the ILG from a naïve and a Con A-injected rabbit stained with H&E.

FIG. 4 illustrates that PS suppresses dry eye disease in rabbits. DED was induced by three sets of Con A injections as in Methods in two groups of rabbits that were treated with either vehicle or PS for three weeks and compared to a control naïve group (n=8-10 eyes/group). PS normalized TBUT, osmolarity and tear lactoferrin levels in contrast to vehicle. STT was improved by PS but the difference from vehicle did not reach statistical significance. Values=mean±SEM.

FIG. 5 illustrates a comparison of the effect on DED in rabbits of PS to two ophthalmic NSAIDs. Four groups of rabbits with DED induced by Con A were treated with vehicle or PS or ketorolac or diclofenac daily for one week as in Methods. A naïve group was used as a control. The values of TBUT, osmolarity and STT were comparable at baseline. The histograms depict the results for these three parameters on day 5. The results from the three test drugs were compared to those from the vehicle group; the three statistically significant differences are shown; all others were not significant. The vehicle group values were significantly different from the naïve group (not shown). Values=mean±SEM

FIGS. 6A and 6B illustrate that PS suppresses the activation of NF-κB and MAPKs. In FIG. 6A, NF-κB activation was determined by EMSA in cultured human conjunctival cells stimulated with TNFα (top) and in the ILG of rabbits with Con A-induced DED and treated for one week with either vehicle or PS (bottom). In FIG. 6B, immunoblots detecting the activation of MAPKs by phosphorylation in cultured human conjunctival cells treated with PS at the indicated concentrations for 3.5 h. Loading control: β-actin.

FIGS. 7A and 7B illustrate that PS suppresses cytokine levels in cultured conjunctival cells and the ILG of rabbits with DED. In FIG. 7A, human conjunctival cells were treated for 24 h with PS at 1×IC₅₀ (TNF-α was added to the culture medium at a concentration of 10 ng/ml 2 h after PS). Cytokine levels were determined by ELISA and represent the average of a three samples. In FIG. 7B, IL-1β and IL-8 levels were determined by ELISA in the lacrimal glands of rabbits with Con A-induced DED that were treated with vehicle or PS for one week as previously. Gland tissue was homogenized and ELISA was performed on whole-tissue lysates. n=8 glands/group. Values=mean±SEM

FIGS. 8A and 8B illustrate that PS suppresses the levels and activity of MMPs. In FIG. 8A, the human conjunctival cells were treated with PS at 1×IC₅₀(TNF-α was added to the culture medium at a concentration of 10 ng/ml 2 h after PS. The levels of MMP-1 in the culture medium were determined by ELISA as in Methods (n=3). Values=mean±SEM. In FIG. 8B, two groups of rabbits with Con A-induced DED were treated with vehicle or PS for 1 week as in Methods. Naïve rabbits served as controls. MMP-9 levels in the ILG (top) and the aqueous humor (middle) were determined by ELISA. MMP activity was determined in the cornea of naïve and PS- or ketorolac-treated rabbits with Con A-induced DED as previously. n=8 eyes/group. Values=mean±SEM.

FIGS. 9A and 9B illustrate that PS preserves the levels of PGE₂ in tears and the cornea. In FIG. 9A, PGE₂ levels were determined by ELISA in tears collected on day 7 from naïve rabbits and rabbits with Con A-induced DED treated for 1 week with vehicle or PS. In FIG. 9B, PGE₂ levels were further examined. Upper panel: PGE₂ levels in the tears of naïve rabbits and rabbits with Con A-induced DED treated for 1 h with PS or ketorolac as in Methods. Lower panel: PGE₂ levels in the corneal tissue of naïve rabbits and rabbits with Con A-induced DED treated for 1 week with vehicle or PS or ketorolac or diclofenac. n=8 eyes/group. Values=mean±SEM.

FIGS. 10A and 10B illustrate the ocular analgesic effect of PS. FIG. 10A: One drop of PS 0.5%, vehicle, or lidocaine was applied to one eye of rabbits (n=4/group) and the corneal touch threshold (CTT) was determined using an Eshesiometer. Vehicle had no effect on CTT (not shown; overlaps with the 0 value horizontal line). Values=mean±SEM. FIG. 10B: PS 0.5% in formulations differing in pH as indicated produced different analgesic responses in rabbits. The areas under each curve (AUC), indicted in the figure, that quantify these responses vary by as much as >5 fold. Values are the average of 2; all were within 11% of each other.

FIGS. 11A and 11B illustrates the effect of various concentrations of PS on corneal sensitivity determined by the corneal touch threshold (CTT) assay. The CTT score is expressed as length of filament. Measurements were performed at the indicated time points after a single application of PS as an eye drop. Rabbits with normal or dry eyes were studied (n=6 eyes per group). Dry eyes were induced by Concanavalin A injection as described in the text. The % PS content in each study is shown. The numbers in parentheses indicate the corresponding value of the area under the curve. Values=mean±SEM.

FIGS. 12A and 12B illustrate the effect of various drugs on corneal sensitivity determined by the corneal touch threshold (CTT) assay described herein. Each drug was used in its commercially available form; one eye drop of each was applied. The numbers in parentheses indicate the corresponding value of the area under the curve. Values=mean±SEM.

FIGS. 13A-13D illustrate images of chorioallantoic membrane (CAM) under various conditions where PS markedly decreased new vessel formation in CAM.

FIGS. 14A-14C illustrate the inhibition of angiogenesis in the lacrimal gland of rabbits with DED.

FIGS. 15A and 15B illustrate that PS suppresses ocular inflammation in rabbits. Photographs were obtained 24 h after initiation of treatment. FIG. 15A: Rabbits treated with vehicle show a marked inflammatory reaction, making opening of their eyes difficult due to periorbital edema. FIG. 15B: PS-treated rabbits have minimal or no inflammatory reaction, permitting them to fully open their eyes.

FIGS. 16A and 16B illustrates that PS suppresses the number of inflammatory cells in rabbits. FIGS. 16A and 16B upper panels: the marked inflammatory reaction induced in rabbits by cataract surgery plus LPS, led to a dramatic increase in the number of inflammatory cells in AH in vehicle-treated rabbits, which was prevented by PS. Data are from the four rabbits depicted in FIG. 15. Individual values are the average of the two eyes of each rabbit. FIGS. 16A and 16B lower panel: representative photographs of two implanted lenses removed on day 5. The one from a vehicle-treated rabbit shows an abundance of cells attached to it. Very few cells can be seen in the lens from the PS-treated rabbit.

FIG. 17 illustrates an agar plate with susceptibility discs applied to a S. aureus growth. The growth inhibition zones are evident. Levofloxacin was the antibiotic tested

### DETAILED DESCRIPTION OF THE INVENTION

Unless defined otherwise, all technical and scientific terms used herein have the same meaning as is commonly understood by one of skill in the art to which this invention belongs. All patents and publications referred to herein are incorporated by reference in their entireties.

### Definitions

As used herein, the terms “administer,” “administration” or “administering” refer to (1) providing, giving, dosing, and/or prescribing by either a health practitioner or his authorized agent or under his or her direction according to the disclosure; and/or (2) putting into, taking or consuming by the mammal, according to the disclosure.

The terms “co-administration,” “co-administering,” “administered in combination with,” “administering in combination with,” “simultaneous,” and “concurrent,” as used herein, encompass administration of two or more active pharmaceutical ingredients to a subject so that both active pharmaceutical ingredients and/or their metabolites are present in the subject at the same time. Co-administration includes simultaneous administration in separate compositions, administration at different times in separate compositions, or administration in a composition in which two or more active pharmaceutical ingredients are present. Simultaneous administration in separate compositions and administration in a composition in which both agents are present are preferred.

The term “compound with reduced risk of corneal melt” refers to compounds that are less likely to cause corneal melt in a patient being treated when compared to an NSAID known to cause corneal melt (e.g., diclofenac (see, e.g., Julianne, C. et al. “Corneal Melting Associated with Use of Topical Nonsteroidal Anti-Inflammatory Drugs after Ocular Surger,” (2000) 118:1129-1132)) at about the same dosage. The compounds of formula (I) and formula (II) are compounds with reduced risk of corneal melt.

The terms “active pharmaceutical ingredient” and “drug” include the compounds described herein and, more specifically, the compounds described by formula (I) or formula (II).

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

The terms “QD,” “qd,” or “q.d.” mean quaque die, once a day, or once daily. The terms “BID,” “bid,” or “b.i.d.” mean bis in die, twice a day, or twice daily. The terms “TID,” “tid,” or “t.i.d.” mean ter in die, three times a day, or three times daily. The terms “QID,” “qid,” or “q.i.d.” mean quater in die, four times a day, or four times daily.

The term “pharmaceutically acceptable salt” refers to salts derived from a variety of organic and inorganic counter ions known in the art. Pharmaceutically acceptable acid addition salts can be formed with inorganic acids and organic acids. Preferred inorganic acids from which salts can be derived include, for example, hydrochloric acid, hydrobromic acid, sulfuric acid, nitric acid and phosphoric acid. Preferred organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese and aluminum. Organic bases from which salts can be derived include, for example, primary, secondary, and tertiary amines, substituted amines including naturally occurring substituted amines, cyclic amines and basic ion exchange resins. Specific examples include isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts. The term “cocrystal” refers to a molecular complex derived from a number of cocrystal formers known in the art. Unlike a salt, a cocrystal typically does not involve hydrogen transfer between the cocrystal and the drug, and instead involves intermolecular interactions, such as hydrogen bonding, aromatic ring stacking, or dispersive forces, between the cocrystal former and the drug in the crystal structure.

“Pharmaceutically acceptable carrier” or “pharmaceutically acceptable excipient” is intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the invention is contemplated. Additional active pharmaceutical ingredients, such as other drugs disclosed herein, can also be incorporated into the described compositions and methods.

As used herein, the terms “treat,” “treatment,” and/or “treating” may refer to the management of a disease, disorder, or pathological condition, or symptom thereof with the intent to cure, ameliorate, stabilize, and/or control the disease, disorder, pathological condition or symptom thereof. Regarding control of the disease, disorder, or pathological condition more specifically, “control” may include the absence of condition progression, as assessed by the response to the methods recited herein, where such response may be complete (e.g., placing the disease in remission) or partial (e.g., lessening or ameliorating any symptoms associated with the condition).

As used herein, the terms “modulate” and “modulation” refer to a change in biological activity for a biological molecule (e.g., a protein, gene, peptide, antibody, and the like), where such change may relate to an increase in biological activity (e.g., increased activity, agonism, activation, expression, upregulation, and/or increased expression) or decrease in biological activity (e.g., decreased activity, antagonism, suppression, deactivation, downregulation, and/or decreased expression) for the biological molecule.

As used herein, the term “prodrug” refers to a derivative of a compound described herein, the pharmacologic action of which results from the conversion by chemical or metabolic processes in vivo to the active compound. Prodrugs include compounds wherein an amino acid residue, or a polypeptide chain of two or more (e.g., two, three or four) amino acid residues is covalently joined through an amide or ester bond to a free amino, hydroxyl or carboxylic acid group of formula (I) or formula (II). The amino acid residues include but are not limited to the 20 naturally occurring amino acids commonly designated by one or three letter symbols but also include, for example, 4-hydroxyproline, hydroxylysine, desmosine, isodesmosine, 3-methylhistidine, beta-alanine, gamma-aminobutyric acid, citrulline, homocysteine, homoserine, ornithine and methionine sulfone. Additional types of prodrugs are also encompassed. For instance, free carboxyl groups can be derivatized as amides or alkyl esters (e.g., methyl esters and acetoxy methyl esters). Prodrug esters as employed herein includes esters and carbonates formed by reacting one or more hydroxyls of compounds of the method of the invention with alkyl, alkoxy, or aryl substituted acylating agents employing procedures known to those skilled in the art to generate acetates, pivalates, methylcarbonates, benzoates and the like. As further examples, free hydroxyl groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxyl and amino groups are also included, as are carbonate prodrugs, sulfonate prodrugs, sulfonate esters and sulfate esters of hydroxyl groups. Free amines can also be derivatized to amides, sulfonamides or phosphonamides. All of the stated prodrug moieties may incorporate groups including but not limited to ether, amine and carboxylic acid functionalities. Moreover, any compound that can be converted in vivo to provide the bioactive agent (e.g., a compound of formula (I) or formula (II)) is a prodrug within the scope of the invention. Various forms of prodrugs are well known in the art. A comprehensive description of pro drugs and prodrug derivatives are described in: (a) The Practice of Medicinal Chemistry, Camille G. Wermuth et al., (Academic Press, 1996); (b) Design of Prodrugs, edited by H. Bundgaard, (Elsevier, 1985); (c) A Textbook of Drug Design and Development, P. Krogsgaard-Larson and H. Bundgaard, eds., (Harwood Academic Publishers, 1991). In general, prodrugs may be designed to improve the penetration of a drug across biological membranes in order to obtain improved drug absorption, to prolong duration of action of a drug (slow release of the parent drug from a prodrug, decreased first-pass metabolism of the drug), to target the drug action (e.g. organ or tumor-targeting, lymphocyte targeting), to modify or improve aqueous solubility of a drug (e.g., i.v. preparations and eyedrops), to improve topical drug delivery (e.g. dermal and ocular drug delivery), to improve the chemical/enzymatic stability of a drug, or to decrease off-target drug effects, and more generally in order to improve the therapeutic efficacy of the compounds utilized in the invention.

Unless otherwise stated, the chemical structures depicted herein are intended to include compounds which differ only in the presence of one or more isotopically enriched atoms. For example, compounds where one or more hydrogen atoms is replaced by deuterium or tritium, or wherein one or more carbon atoms is replaced by ¹³C- or ¹⁴C-enriched carbons, are within the scope of this invention.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 15%, preferably from 0% to 10%, more preferably from 0% to 5% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

“Isomers” are different compounds that have the same molecular formula. “Stereoisomers” are isomers that differ only in the way the atoms are arranged in space—i.e., having a different stereochemical configuration. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon can be specified by either (R) or (S). Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry, as (R) or (S). The present chemical entities, pharmaceutical compositions and methods are meant to include all such possible isomers, including racemic mixtures, optically pure forms and intermediate mixtures. Optically active (R)- and (S)-isomers can be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers.

“Enantiomeric purity” as used herein refers to the relative amounts, expressed as a percentage, of the presence of a specific enantiomer relative to the other enantiomer. For example, if a compound, which may potentially have an (R)- or an (S)-isomeric configuration, is present as a racemic mixture, the enantiomeric purity is about 50% with respect to either the (R)- or (S)-isomer. If that compound has one isomeric form predominant over the other, for example, 80% (S)-isomer and 20% (R)-isomer, the enantiomeric purity of the compound with respect to the (S)-isomeric form is 80%. The enantiomeric purity of a compound can be determined in a number of ways known in the art, including but not limited to chromatography using a chiral support, polarimetric measurement of the rotation of polarized light, nuclear magnetic resonance spectroscopy using chiral shift reagents which include but are not limited to lanthanide containing chiral complexes or Pirkle's reagents, or derivatization of a compounds using a chiral compound such as Mosher's acid followed by chromatography or nuclear magnetic resonance spectroscopy.

In preferred embodiments, the enantiomerically enriched composition has a higher potency with respect to therapeutic utility per unit mass than does the racemic mixture of that composition. Enantiomers can be isolated from mixtures by methods known to those skilled in the art, including chiral high pressure liquid chromatography (HPLC) and the formation and crystallization of chiral salts; or preferred enantiomers can be prepared by asymmetric syntheses. See, for example, Jacques, et al., Enantiomers, Racemates and Resolutions, Wiley Interscience, New York (1981); E. L. Eliel, Stereochemistry of Carbon Compounds, McGraw-Hill, New York (1962); and E. L. Eliel and S. H. Wilen, Stereochemistry of Organic Compounds, Wiley-Interscience, New York (1994).

The terms “enantiomerically enriched” and “non-racemic,” as used herein, refer to compositions in which the percent by weight of one enantiomer is greater than the amount of that one enantiomer in a control mixture of the racemic composition (e.g., greater than 1:1 by weight). For example, an enantiomerically enriched preparation of the (S)-enantiomer, means a preparation of the compound having greater than 50% by weight of the (S)-enantiomer relative to the (R)-enantiomer, such as at least 75% by weight, or such as at least 80% by weight. In some embodiments, the enrichment can be significantly greater than 80% by weight, providing a “substantially enantiomerically enriched” or a “substantially non-racemic” preparation, which refers to preparations of compositions which have at least 85% by weight of one enantiomer relative to other enantiomer, such as at least 90% by weight, or such as at least 95% by weight. The terms “enantiomerically pure” or “substantially enantiomerically pure” refers to a composition that comprises at least 98% of a single enantiomer and less than 2% of the opposite enantiomer.

“Moiety” refers to a specific segment or functional group of a molecule. Chemical moieties are often recognized chemical entities embedded in or appended to a molecule.

“Tautomers” are structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g., in solution), a chemical equilibrium of tautomers can be reached. An example of tautomerization is keto-enol tautomerization. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

“Protecting group” is intended to mean a group that selectively blocks one or more reactive sites in a multifunctional compound such that a chemical reaction can be carried out selectively on another unprotected reactive site and the group can then be readily removed or deprotected after the selective reaction is complete. A variety of protecting groups are disclosed, for example, in T. H. Greene and P. G. M. Wuts, Protective Groups in Organic Synthesis, Third Edition, John Wiley & Sons, New York (1999).

“Solvate” refers to a compound in physical association with one or more molecules of a pharmaceutically acceptable solvent.

Compounds of the invention also include crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof. “Crystalline form” and “polymorph” are intended to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to.

For the avoidance of doubt, it is intended herein that particular features (for example integers, characteristics, values, uses, diseases, formulae, compounds or groups) described in conjunction with a particular aspect, embodiment or example of the invention are to be understood as applicable to any other aspect, embodiment or example described herein unless incompatible therewith. Thus such features may be used where appropriate in conjunction with any of the definition, claims or embodiments defined herein. All of the features disclosed in this specification (including any accompanying claims, abstract and drawings), and/or all of the steps of any method or process so disclosed, may be combined in any combination, except combinations where at least some of the features and/or steps are mutually exclusive. The invention is not restricted to any details of any disclosed embodiments. The invention extends to any novel one, or novel combination, of the features disclosed in this specification (including any accompanying claims, abstract and drawings), or to any novel one, or any novel combination, of the steps of any method or process so disclosed.

Moreover, as used herein, the term “about” means that dimensions, sizes, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

Furthermore, the transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All embodiments of the invention can, in the alternative, be more specifically defined by any of the transitional terms “comprising,” “consisting essentially of,” and “consisting of.”

### Methods of Treating Diseases and Conditions of the Eye

The compounds and compositions described herein can be used in methods for treating diseases of the eye. In some embodiments, the diseases of the eye that are treated by the compounds, compositions, methods, and kits described herein include dry eye disease and retinopathy. In some embodiments, retinopathy may include the diseases of diabetic retinopathy, retinopathy of prematurity, VEGF retinopathy, age related macular degeneration, retinal vein occlusion, and/or hypertensive retinopathy. In certain embodiments, retinopathy may be diabetic retinopathy.

Dry eye disease (DED) is a multi-factorial disease of the ocular surface characterized by loss of homeostasis of the tear film and accompanied by ocular symptoms. The tear film in DED is abnormal because of one or more of three reasons: tear production is decreased; tear evaporation is increased; or the mucus or lipids of the tear are abnormal. The clinical manifestations of DED can vary in severity from very mild to the point that they decrease the ability to perform activities requiring visual attention such as reading and driving, seriously affecting the patient's quality of life. Given its worldwide distribution and the lack of a single definitive test or consensus of criteria for its diagnosis, prevalence figures for DED vary. The best estimate of its prevalence is 15% (17.9% for women and 10.5% for men); some authors consider even 15% an underestimate.

DED is an inflammatory disease whose pathogenesis is under extensive study. For example, dysfunction of the tear glands, chronic irritative stress or systemic autoimmune diseases can lead to ocular inflammation. In turn, inflammation causes dysfunction or death of cells responsible for tear secretion establishing a vicious cycle, which, regardless of the initiating insult, leads to ocular surface disease. The important contributors to the inflammatory process in DED are: (1) activation of pro-inflammatory cytokines; tear hyperosmolarity, which stimulates inflammatory mediators through MAPKs; (2) matrix metalloproteinases (MMPs), which lyse components of the corneal epithelial basement membrane and tight junction proteins; (3) chemokines, which recruit nearby responsive cells; and (4) T cells, which can amplify the cascade by attracting inflammatory cells, e.g., in Sjögren's syndrome.

The treatment of DED depends on its clinical severity. The symptoms of very mild disease are often treated with artificial tears, which provide partial relief but do not suppress inflammation. Advanced disease is managed with the immunosuppressant cyclosporine, the recently approved integrin antagonist lifitegrast, punctal plugs, or rarely corticosteroids. Non-steroidal anti-inflammatory drugs (NSAIDs) have no role in DES.

In an embodiment, the invention includes a method for treating dry eye disease in a patient in need thereof, the method comprising administering to the patient a therapeutically effective amount of a compound of formula I or formula II, or a pharmaceutically acceptable salt thereof.

In some embodiments, the compound may be a compound of formula I or a pharmaceutically acceptable salt thereof.

In some embodiments, the methods for the treatment of dry eye disease may include the administration of a therapeutically effective amount of an additional active agent. In some embodiments, the additional active agent may include one or more of an antibiotic, cyclosporine, and lifitegrast.

Diabetic retinopathy refers to retinal changes that occur in patients with diabetes mellitus. These changes affect the small blood vessels of the retina and can lead to vision loss through several different pathways. Macular edema, defined as retinal thickening and edema involving the macula can occur at any stage of diabetic retinopathy. Diabetic retinopathy is one of the commonest causes of vision loss. Vascular endothelial growth factor (VEGF) is secreted by ischemic retina. VEGF leads to (a) increased vascular permeability resulting in retinal swelling/edema and (b) angiogenesis-new blood vessel formation. Agents that suppress VEGF can control diabetic retinopathy.

In addition to diabetic retinopathy, several other ocular diseases are characterized by abnormal vascular phenomena that are predominantly dependent on VEGF. Given the role of VEGF in these disorders, controlling VEGF is an approach to their prevention and treatment. Prominent among them is age-related macular degeneration (AMD), a degenerative disease of the central portion of the retina (the macula) that results primarily in loss of central vision. Central vision is required for activities such as driving, reading, watching television, and performing activities of daily living. AMD is classified as dry (atrophic) or wet (neovascular or exudative) for clinical purposes. Wet AMD, also referred to as choroidal neovascularization is characterized by growth of abnormal vessels into the subretinal space, usually from the choroidal circulation and less frequently from the retinal circulation. These abnormal blood vessels leak, leading to collections of subretinal fluid and/or blood beneath the retina.

Retinal vein occlusion (RVO) is an important cause of visual loss among older adults throughout the world. An important component of RVO which is also a therapeutic target for this entity are its secondary complications that affect vision, including macular edema, retinal neovascularization, and anterior segment neovascularization. VEGF pays a crucial role in these vision-determining complications. Patients with severe (ischemic) central retinal vein occlusion are at particularly high risk for neovascular glaucoma, often within the first few months of diagnosis, and should be observed at least monthly for development of anterior segment neovascularization during this period. Indeed, patients with severe (ischemic) central retinal vein occlusion are at particularly high risk for neovascular glaucoma, and are observed closely for development of anterior segment neovascularization. VEGF inhibitors in patients with RVO are hypothesized to limit macular edema and improve vision by decreasing vascular permeability.

In an embodiment, the invention includes a method for treating diabetic retinopathy in a patient in need thereof, the method comprising administering to the patient a therapeutically effective amount of a compound of formula I or formula II, or a pharmaceutically acceptable salt thereof.

In an embodiment, the invention includes a method of treating an ophthalmic condition in a patient in need thereof, wherein the ophthalmic condition is selected from the group consisting of dry eye disease and retinopathy, the method comprising administering to the patient a therapeutically effective amount of a compound with reduced risk of corneal melt of formula I or formula II, or a pharmaceutically acceptable salt thereof.

In an embodiment, the invention includes a composition for treating an ophthalmic condition in a patient in need thereof, wherein the ophthalmic condition is selected from the group consisting of dry eye disease and retinopathy, the composition comprising a therapeutically effective amount of a compound with reduced risk of corneal melt of formula I or formula II, or a pharmaceutically acceptable salt thereof, and a pharmaceutically acceptable carrier.

In some embodiments, the compound may be a compound of formula I or a pharmaceutically acceptable salt thereof.

In some embodiments, the methods for the treatment of diabetic retinopathy may include the administration of a therapeutically effective amount of an additional active agent. In some embodiments, the additional active agent may include one or more of an antibiotic, cyclosporine, and lifitegrast.

In some embodiments, the antibiotic the antibiotic may include one or more of tetracycline, tobramycin, chlortetracycline, bacitracin, neomycin, polymyxin, gramicidin, oxytetracycline, chloramphenicol, gentamycin, and erythromycin. Other antibiotics include aminoglycoside, ampicillin, carbenicillin, cefazolin, cephalosporin, chloramphenicol, clindamycin, everninomycin, gentamycin, kanamycin, lipopeptides, methicillin, nafcillin, novobiocia, oxazolidinones, penicillin, quinolones, rifampin, streptogramins, streptomycin, sulfamethoxazole, sulfonamide, trimethoprim, and vancomycin.

In some embodiments, the antibiotic may include neomycin sulfate or polymyxin B sulfate.

In some embodiments, the methods described herein may include the administration of an additional compound for treating an ophthalmic condition, the additional compound may comprise one or more of the compounds disclosed in U.S. Pat. No. 8,236,820 and/or U.S. Patent Application Nos. 2009/0099137, 2013/0225529, and 2014/0315834, the entireties of which are incorporated herein by reference.

Efficacy of the methods, compounds, and combinations of compounds described herein in treating, preventing and/or managing the indicated diseases or disorders can be tested using various animal models known in the art.

### Non-Steroidal Anti-Inflammatory Drug (NSAID) Derivative Compounds

In an embodiment, the compounds described herein may be NSAID derivative compounds.

NSAIDs are not used in the treatment of DED for two reasons. First, there is no evidence that they would be efficacious. Second, they are associated with prohibitive ocular side effects, most notably corneal melt. Indeed, NSAIDs are contraindicated in patients with DED.

The most dangerous complication of topical ophthalmic NSAIDs is corneal melt. Corneal melt is a condition where the corneal epithelium is severely damaged or lost and is accompanied by thinning of the corneal stroma, which consists mainly of collagen. Progressive thinning of the stroma may result in perforation of the eye that can lead to loss of vision through major refractive errors or even to loss of the eye itself from subsequent complications such as infection. Corneal melts typically occur after ocular surgery and in the setting of inflammation or other insult to the corneal surface. However, corneal melt may occur in the absence of inflammation or other insult.

In general, opinion leaders recommend extreme care in the use of NSAIDs in ophthalmology and do not recommend their use in DED because the risk of corneal melt is increased as the cornea is already compromised by DED.

In an embodiment, the compounds described herein include the NSAID derivative compounds of Formula I and Formula II, or the pharmaceutically acceptable salts thereof.

In an embodiment, the compound of the invention may include the compound of Formula I:

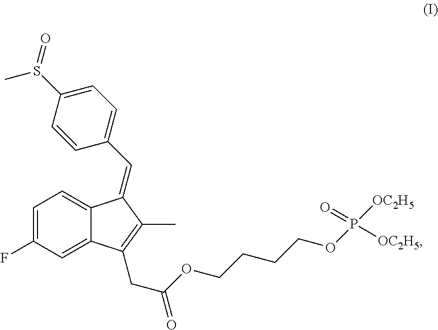 [SMILES: CCOP(=O)(OCC)OCCCCOC(=O)CC1=C(C)/C(=C/c2ccc(S(C)=O)cc2)c2ccc(F)cc21]

or a pharmaceutically acceptable salt thereof.

In an embodiment, the compound of the invention may include the compound of Formula II:

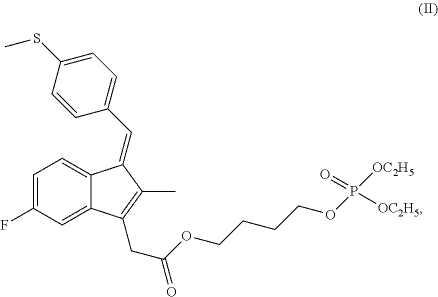 [SMILES: CCOP(=O)(OCC)OCCCCOC(=O)CC1=C(C)/C(=C/c2ccc(SC)cc2)c2ccc(F)cc21]

or a pharmaceutically acceptable salt thereof.

The compounds of formulas I and II are described in U.S. Pat. No. 8,236,820, the entirety of which is incorporated herein by reference.

For example, the Formula I compound (PS) is a derivative of the NSAID sulindac. Thus, one may anticipate that it would also be either ineffective or contraindicated in the treatment of DED.

In some embodiments, the compounds of Formula I and Formula II may penetrate one or more of the cornea, sclera, and conjunctiva to contact the retina.

However, PS is efficacious and safe in the treatment of DED. In particular, PS, when administered at doses and over time periods effective to treat DED, does not cause corneal melt.

PS is also efficacious and safe as an analgesic for eye pain. Since PS is not behaving as a conventional NSAID, one would expect that PS would lose the beneficial analgesic properties displayed by ophthalmic NSAIDs such as ketorolac and others. However, PS displays a strong analgesic effect in ocular tissues.

### Pharmaceutical Compositions

In an embodiment, the invention provides a pharmaceutical composition for use in the treatment of the diseases and conditions described herein.

The pharmaceutical compositions are typically formulated to provide a therapeutically effective amount of a compound of formula (I) or formula (II), as described herein, or a pharmaceutically acceptable salt, solvate, or hydrate thereof, as the active ingredient.

In some embodiments, the pharmaceutical compositions are formulated as emulsions able to provide a therapeutically effective amount of a compound of formula (I) or formula (II), as described herein, or a pharmaceutically acceptable salt, solvate, or hydrate thereof, as the active ingredient.

In some embodiments, the pharmaceutical compositions described herein may include an additional active agent. In some embodiments, the additional active agent may include one or more of an antibiotic, cyclosporine, and lifitegrast.

Typically, the pharmaceutical compositions also comprise one or more pharmaceutically acceptable excipients, carriers, including inert solid diluents and fillers, diluents, including sterile aqueous solution and various organic solvents, permeation enhancers, solubilizers and adjuvants.

The pharmaceutical compositions described above are preferably for use in the treatment of an ophthalmic condition or disease, such as dry eye disease or diabetic retinopathy.

In some embodiments, the concentration of a compound of formula (I) or formula (II) provided in the pharmaceutical compositions of the invention is less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v of the pharmaceutical composition.

In some embodiments, the concentration of a compound of formula (I) or formula (II) provided in the pharmaceutical compositions of the invention is independently greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition.

In some embodiments, the concentration of a compound of formula (I) or formula (II) provided in the pharmaceutical compositions of the invention is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12% or about 1% to about 10% w/w, w/v or v/v of the pharmaceutical composition.

In some embodiments, the concentration of a compound of formula (I) or formula (II) provided in the pharmaceutical compositions of the invention is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v of the pharmaceutical composition.

In some embodiments, the amount of a compound of formula (I) or formula (II) provided in the pharmaceutical compositions of the invention is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of a compound of formula (I) or formula (II) provided in the pharmaceutical compositions of the invention is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5 g, 3 g, 3.5 g, 4 g, 4.5 g, 5 g, 5.5 g, 6 g, 6.5 g, 7 g, 7.5 g, 8 g, 8.5 g, 9 g, 9.5 g, or 10 g.

Each of the compounds provided according to the invention is effective over a wide dosage range. For example, in the treatment of adult humans, dosages independently ranging from 0.01 to 1000 mg, from 0.5 to 100 mg, from 1 to 50 mg per day, and from 5 to 40 mg per day are examples of dosages that may be used. The exact dosage will depend upon the route of administration, the form in which the compound is administered, the gender and age of the subject to be treated, the body weight of the subject to be treated, and the preference and experience of the attending physician.

Described below are non-limiting pharmaceutical compositions and methods for preparing the same.

### Pharmaceutical Compositions for Topical Delivery

In preferred embodiments, the invention provides a pharmaceutical composition for topical delivery containing a compound of formula (I) or formula (II) described herein, and a pharmaceutical excipient suitable for topical delivery.

Compositions of the invention can be formulated into preparations in solid, semi-solid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The compositions described herein may be formulated for administration topically to the eye and surrounding tissues, particularly to the inner surface of the eye and the inner surface of the eyelids (including e.g. cornea, conjunctiva and sclera). Such compositions, for example, may be formulated for instillation administration, administration into conjunctival sac and conjunctival administration. In particular, the compositions described herein may be formulated as eye drops. Such eye drop formulations may include a liquid or semisolid pharmaceutical composition adapted to administration to the eye. A typical example of an eye drop composition is an ophthalmic solution to be administered dropwise to the eye. In some embodiments, an eye drop composition is an ophthalmic emulsion to be administered dropwise to the eye.

In certain embodiments, the compositions of the invention are in the form of eye drops. In some embodiments, the size of the drop is between about 10 and about 100 μL. The drop size may be greater than about 10 greater than about 20 greater than about 30 greater than about 40 greater than about 50 greater than about 60 greater than about 70 greater than about 80 greater than about 90 or greater than about 100 μL. The drop size may be less than about 10 less than about 20 less than about 30 less than about 40 less than about 50 less than about 60 less than about 70 less than about 80 less than about 90 or less than about 100 μL.

The pharmaceutical compositions also may comprise suitable solid or gel phase carriers or excipients, which are compounds that allow increased penetration of, or assist in the delivery of, therapeutic molecules across the membranes of the eye, including, but not limited to, the cornea, conjunctiva, and sclera. There are many of these penetration-enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

In some embodiments, the compositions described herein may include liquid formulations, semi-solid formulations, and multicompartment formulations. In some embodiments, the compositions described herein may include emulsions.

In an embodiment, the compositions described herein may be liquid formulations that may include an ophthalmic solution of PS and/or a microemulsion of PS. Active pharmaceutical ingredients (APIs) for which microemulsions have been developed include cyclosporine A and flurbiprofen axetil. Successful approaches to extend the contact time of liquid dosage forms with ocular tissues and to increase the tissue uptake of the API include the use of excipients that increase viscosity, enhance penetration, or cyclodextrins. Cyclodextrins are cyclic oligosaccharides that form inclusion complexes with APIs that increase the aqueous solubility and bioavailability of hydrophobic APIs. In an embodiment, the compositions described herein may include β-cyclodextrin and a therapeutically effective amount of PS.

In an embodiment, the invention includes a composition for treating an ophthalmic condition in a patient in need thereof, wherein the ophthalmic condition is selected from the group consisting of dry eye disease and retinopathy, the composition comprising a therapeutically effective amount of a compound of formula I or formula II, or a pharmaceutically acceptable salt thereof. In some embodiments, the compositions described herein include a pharmaceutically acceptable carrier. In some embodiments, the compositions described herein include one or more of a solubilizing agent, an alcohol, an acid, and a preservative. In some embodiments, the compositions described herein include water.

In some embodiments, the compositions described herein include a solubilizing agent and an alcohol. In some embodiments, the compositions described herein include a solubilizing agents and an acid. In some embodiments, the compositions described herein include a solubilizing agents and a preservative. In some embodiments, the compositions described herein include a solubilizing agent, an alcohol, and an acid. In some embodiments, the compositions described herein include a solubilizing agent, an alcohol, an acid, and a preservative.

In some embodiments, the compositions of the invention may include a compound of formula I or formula II, or a pharmaceutically acceptable salt thereof, in an amount, by weight, of about 0.5% to about 75%, or about 0.5% to about 70%, or about 0.5% to about 65%, or about 0.5% to about 60%, or about 0.5% to about 55%, or about 0.5% to about 50%, or about 0.5% to about 45%, or about 0.5% to about 40%, or about 0.5% to about 35%, or about 0.5% to about 30%, or about 0.5% to about 25%, or about 0.5% to about 20%, or about 0.5% to about 15%, or about 0.5% to about 10%, or about 0.5% to about 9%, or about 0.5% to about 8%, or about 0.5% to about 7%, or about 0.5% to about 6%, or about 0.5% to about 5%, or about 0.5% to about 4%, or about 0.5% to about 3%, or about 0.5% to about 2%, or about 0.5% to about 1%.

In some embodiments, the solubilizing agent is vitamin E TPGS (d-α-tocopheryl polyethylene glycol 1000 succinate). In some embodiments, the compositions described herein include a solubilizing agent in an amount, by weight, of about 0.5% to about 75%, or about 1% to about 70%, or about 1% to about 65%, or about 1% to about 60%, or about 1% to about 55%, or about 1% to about 50%, or about 1% to about 45%, or about 1% to about 40%, or about 1% to about 35%, or about 1% to about 30%, or about 1% to about 25%, or about 1% to about 20%, or about 1% to about 15%, or about 1% to about 10%, or about 1% to about 5%.

In some embodiments, the alcohol is a sugar alcohol, such as mannitol. In some embodiments, the compositions described herein include an alcohol in an amount by weight, of about 0.5% to about 75%, or about 0.5% to about 70%, or about 0.5% to about 65%, or about 0.5% to about 60%, or about 0.5% to about 55%, or about 0.5% to about 50%, or about 0.5% to about 45%, or about 0.5% to about 40%, or about 0.5% to about 35%, or about 0.5% to about 30%, or about 0.5% to about 25%, or about 0.5% to about 20%, or about 0.5% to about 15%, or about 0.5% to about 10%, or about 0.5% to about 9%, or about 0.5% to about 8%, or about 0.5% to about 7%, or about 0.5% to about 6%, or about 0.5% to about 5%, or about 0.5% to about 4%, or about 0.5% to about 3%, or about 0.5% to about 2%, or about 0.5% to about 1%.

In some embodiments, the acid is boric acid. In some embodiments, the compositions described herein include an acid in an amount, by weight, of about 0.5% to about 75%, or about 0.5% to about 70%, or about 0.5% to about 65%, or about 0.5% to about 60%, or about 0.5% to about 55%, or about 0.5% to about 50%, or about 0.5% to about 45%, or about 0.5% to about 40%, or about 0.5% to about 35%, or about 0.5% to about 30%, or about 0.5% to about 25%, or about 0.5% to about 20%, or about 0.5% to about 15%, or about 0.5% to about 10%, or about 0.5% to about 9%, or about 0.5% to about 8%, or about 0.5% to about 7%, or about 0.5% to about 6%, or about 0.5% to about 5%, or about 0.5% to about 4%, or about 0.5% to about 3%, or about 0.5% to about 2%, or about 0.5% to about 1%.

In some embodiments, the preservative is polyquaternium-1 (polyquad). In some embodiments, the compositions described herein include a preservative in an amount, by weight, of about 0.001% to about 5%, or about 0.001% to about 4%, or about 0.001% to about 3%, or about 0.001% to about 2%, or about 0.001% to about 1%, or about 0.001% to about 0.5%, or about 0.001% to about 0.1%, or about 0.001% to about 0.009%, or about 0.001% to about 0.008%, or about 0.007%, or about 0.001% to about 0.006%, or about 0.001% to about 0.005%.

In an embodiment, the compositions described herein may include a therapeutically effective amount of PS and one or more of a solubilizing agent (e.g., vitamin E TPGS (d-α-tocopheryl polyethylene glycol 1000 succinate)), a sugar alcohol (e.g., mannitol), an acid (e.g., boric acid), and a preservative (e.g., polyquaternium-1 (polyquad)). In some embodiments, such formulations may be used to deliver PS to the retina following topical administration to the eye. In some embodiments, such formulations may be used to deliver PS to the retina in an amount sufficient to treat a retinopathy (i.e., a therapeutically effective amount).

In an embodiment, the compositions described herein may include, by weight, about 0.5% to about 10% PS and one or more of about 0% to about 25% vitamin E TPGS (d-α-tocopheryl polyethylene glycol 1000 succinate), about 0% to about 10% mannitol, about 0% to about 10% boric acid, and about 0% to about 1% polyquaternium-1 (polyquad).

In an embodiment, the compositions described herein may include, by weight, greater than 0.5% PS and one or more of greater than 5% vitamin E TPGS (d-α-tocopheryl polyethylene glycol 1000 succinate), greater than 0.5% mannitol, greater than 0.5% boric acid, and greater than 0.001% polyquaternium-1 (polyquad).

In an embodiment, the compositions described herein may include, by weight, less than 10% PS and one or more of less than 25% vitamin E TPGS (d-α-tocopheryl polyethylene glycol 1000 succinate), less than 10% mannitol, less than 10% boric acid, and less than 1% polyquaternium-1 (polyquad).

In an embodiment, the compositions described herein may include, by weight, about 3.5% PS and one or more of about 16% vitamin E TPGS (d-α-tocopheryl polyethylene glycol 1000 succinate), about 3.18% mannitol, about 1.2% boric acid, and about 0.005% polyquaternium-1 (polyquad).

In an embodiment, the compositions described herein may be semi-solid formulations that include a gel or viscous excipient and PS. Such semi-solid formulations include high viscosity formulations that increase bioavailability by increasing the residence time of the API in the precorneal area. In situ gels are viscous liquids that undergo sol-to-gel transitions upon ocular application because of changes in pH, temperature or electrolyte concentration. Gelling excipients with favorable mucoadhesive properties further increase the residence time. Polymers or gelling excipients employed in developing these drug forms include gellan gum, sodium alginate, poloxamer, and cellulose acetate phthalate. In an embodiment, the compositions described herein may include a PS thermogel using poloxamer 407 or gellan gum, and comprising a therapeutically effective amount of PS.

In some embodiments, the compositions described herein may include a gelling excipient, such as gellan gum or sodium alginate. In some embodiments, the compositions described herein include a gelling excipient in an amount, by weight, of about 0.5% to about 20%, or about 0.1% to about 15%, or about 0.1% to about 10%, or about 0.1% to about 9%, or about 0.1% to about 8%, or about 0.1% to about 7%, or about 0.1% to about 6%, or about 0.1% to about 5%, or about 0.1% to about 4%, or about 0.1% to about 3%, or about 0.1% to about 2%, or about 0.1% to about 1%, or about 0.1% to about 0.9%, or about 0.1% to about 0.8%, or about 0.1% to about 0.7%, or about 0.1% to about 0.6%, or about 0.1% to about 0.5%.

In some embodiments, the compositions described herein may include a poloxamer. In some embodiments, the compositions described herein include a poloxamer in an amount, by weight, of about 1% to about 75%, or about 1% to about 70%, or about 1% to about 65%, or about 1% to about 60%, or about 1% to about 55%, or about 1% to about 50%, or about 1% to about 45%, or about 1% to about 40%, or about 1% to about 35%, or about 1% to about 30%, or about 1% to about 25%, or about 1% to about 20%, or about 1% to about 15%, or about 1% to about 10%, or about 1% to about 9%, or about 1% to about 8%, or about 1% to about 7%, or about 1% to about 6%, or about 1% to about 5%, or about 1% to about 4%, or about 1% to about 3%, or about 1% to about 2%.

In some embodiments, the compositions described herein include a surfactant, such as Tween 60, Tween 80, or polyoxyl stearate. In some embodiments, the compositions described herein include a surfactant in an amount, by weight, of about 0.01% to about 20%, or about 0.01% to about 15%, or about 0.01% to about 10%, or about 0.01% to about 9%, or about 0.01% to about 8%, or about 0.01% to about 7%, or about 0.01% to about 6%, or about 0.01% to about 5%, or about 0.01% to about 4%, or about 0.01% to about 3%, or about 0.01% to about 2%, or about 0.01% to about 1%, or about 0.01% to about 0.5%, or about 0.01% to about 0.1%, or about 0.01% to about 0.09%, or about 0.01% to about 0.08%, or about 0.07%, or about 0.01% to about 0.06%, or about 0.01% to about 0.05%.

In some embodiments, the compositions described herein include a cyclodextrin, such as (2-hydroxypropyl)-β-cyclodextrin. In some embodiments, the compositions described herein include a cyclodextrin in amount, by weight, of about 0.5% to about 95%, or about 0.5% to about 90%, or about 0.5% to about 85%, or about 0.5% to about 80%, or about 0.5% to about 75%, or about 0.5% to about 70%, or about 0.5% to about 65%, or about 0.5% to about 60%, or about 0.5% to about 55%, or about 0.5% to about 50%, or about 0.5% to about 45%, or about 0.5% to about 40%, or about 0.5% to about 35%, or about 0.5% to about 30%, or about 0.5% to about 25%, or about 0.5% to about 20%, or about 0.5% to about 15%, or about 0.5% to about 10%, or about 0.5% to about 9%, or about 0.5% to about 8%, or about 0.5% to about 7%, or about 0.5% to about 6%, or about 0.5% to about 5%, or about 0.5% to about 4%, or about 0.5% to about 3%, or about 0.5% to about 2%, or about 0.5% to about 1%.

In an embodiment, the compositions described herein may include a therapeutically effective amount of PS and one or more of a gelling excipient (e.g., gellan gum or sodium alginate), a poloxamer, a solubilizing agent (e.g., vitamin E TPGS), a surfactant (e.g., Tween 80 or polyoxyl stearate), a polyether (e.g., a polyethylene glycol, propylene glycol, Cremophor), and a cyclodextrin (e.g., (2-hydroxypropyl)-β-cyclodextrin). In some embodiments, such formulations may allow for delivery of PS to anterior segments of the eye following topical administration. In some embodiments, such formulations may be used to deliver PS to the anterior segments of the eye in an amount sufficient to treat a disease described herein that is associated with such anterior segments of the eye (i.e., a therapeutically effective amount).

As used herein, an amount described as “about 0%,” by weight, is understood to be an amount that is greater than 0%.

In an embodiment, the compositions described herein may include a therapeutically effective amount of PS and one or more of gellan gum, vitamin E TPGS, and a (2-hydroxypropyl)-β-cyclodextrin.

In an embodiment, the compositions described herein may include, by weight, about 0.5% to about 10% PS and one or more of about 0% to about 5% gellan gum, about 0% to about 20% vitamin E TPGS, and about 0% to about 20% (2-hydroxypropyl)-β-cyclodextrin.

In an embodiment, the compositions described herein may include, by weight, greater than 0.5% PS and one or more of greater than 0.1% gellan gum, greater than 1% vitamin E TPGS, and greater than 5% (2-hydroxypropyl)-β-cyclodextrin.

In an embodiment, the compositions described herein may include, by weight, less than 10% PS and one or more of less than 5% gellan gum, less than 20% vitamin E TPGS, less than 20% (2-hydroxypropyl)-β-cyclodextrin.

In an embodiment, the compositions described herein may include, by weight, about 2.4% to about 3% PS and one or more of about 0.5% gellan gum, about 5% vitamin E TPGS, about 10% (2-hydroxypropyl)-β-cyclodextrin.

In an embodiment, the compositions described herein may include, by weight, about 2.4% to about 3% PS and one or more of about 0.4% gellan gum, about 10% vitamin E TPGS, about 5% (2-hydroxypropyl)-β-cyclodextrin.

In an embodiment, the compositions described herein may include a therapeutically effective amount of PS and one or more of sodium alginate, vitamin E TPGS, a (2-hydroxypropyl)-β-cyclodextrin, Tween (e.g., Tween 60 or Tween 80), poly(ethylene glycol) (PEG) (e.g., PEG 400), and polyoxyl stearate.

In an embodiment, the compositions described herein may include a therapeutically effective amount of PS and one or more of propylene glycol, mineral oil, Tween 60 and/or Tween 80, and a (2-hydroxypropyl)-β-cyclodextrin.

In an embodiment, the compositions described herein may include, by weight, between about 0.01% and about 10% of a compound of formula I or formula II, and one or more of between about 0.01% and about 10% propylene glycol, between about 1% and about 25% mineral oil, between about 0.5% and about 10% of one or more of Tween 60 and Tween 80, and between about 1% and about 25% of (2-hydroxypropyl)-β-cyclodextrin (HP-β-CD).

In an embodiment, the compositions described herein may include, by weight, from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12%, or about 1% to about 10% of a compound of formula I or formula II, and one or more of between about 0.01% and about 10% propylene glycol, between about 1% and about 25% mineral oil, between about 0.5% and about 10% of one or more of Tween 60 and Tween 80, and between about 1% and about 25% of (2-hydroxypropyl)-β-cyclodextrin (HP-β-CD).

In an embodiment, the compositions described herein may include, by weight, about 0.5% to about 10% PS and one or more of about 0% to about 5% sodium alginate, about 0% to about 20% vitamin E TPGS, and about 0% to about 20% (2-hydroxypropyl)-β-cyclodextrin.

In an embodiment, the compositions described herein may include, by weight, greater than 0.5% PS and one or more of greater than 0.1% sodium alginate, greater than 1% vitamin E TPGS, and greater than 5% (2-hydroxypropyl)-β-cyclodextrin.

In an embodiment, the compositions described herein may include, by weight, less than 10% PS and one or more of less than 5% sodium alginate, less than 20% vitamin E TPGS, less than 20% (2-hydroxypropyl)-β-cyclodextrin.

In an embodiment, the compositions described herein may include, by weight, about 3% PS and one or more of about 1.5% sodium alginate, about 5% vitamin E TPGS, about 10% (2-hydroxypropyl)-β-cyclodextrin.

In an embodiment, the compositions described herein may include, by weight, about 0.5% to about 10% PS and one or more of about 0% to about 5% sodium alginate, about 0% to about 25% Tween 80, about 0% to about 20% (2-hydroxylpropyl)-β-cyclodextrin, about 0% to about 20% PEG 400, and about 0% to about 10% polyoxyl stearate.

In an embodiment, the compositions described herein may include, by weight, greater than 0.5% PS and one or more of greater than 1% sodium alginate, greater than 1% Tween 80, greater than 1% (2-hydroxylpropyl)-β-cyclodextrin, greater than 1% PEG 400, and greater than 1% polyoxyl stearate.

In an embodiment, the compositions described herein may include, by weight, less than 10% PS and one or more of less than 5% sodium alginate, less than 25% Tween 80, less than 20% (2-hydroxylpropyl)-β-cyclodextrin, less than 20% PEG 400, and less than 10% polyoxyl stearate.

In an embodiment, the compositions described herein may include, by weight, about 3% PS and one or more of about 1.5% sodium alginate, about 15% Tween 80, about 10% (2-hydroxylpropyl)-β-cyclodextrin, about 10% PEG 400, and about 5% polyoxyl stearate.

In an embodiment, the compositions described herein may include, by weight, about 1% to about 5% PS and one or more of about 50% to about 90% (2-hydroxypropyl)-β-cyclodextrin (HP-β-CD), about 0.05% to about 1% cremophor EL (F1), and about 0.5% to about 5% Tween 80 (F2).

In an embodiment, the compositions described herein may include, by weight, about 1% to about 5% PS and one or more of about 50% to about 90% (2-hydroxypropyl)-β-cyclodextrin (HP-β-CD), and about 0.05% to about 1% cremophor EL (F1).

In an embodiment, the compositions described herein may include, by weight, about 1% to about 5% PS and one or more of about 50% to about 90% (2-hydroxypropyl)-β-cyclodextrin (HP-β-CD), and about 0.5% to about 5% Tween 80 (F2).

In an embodiment, the compositions described herein may include, by weight, about 3 to about 4% PS and one or more of about 80% (2-hydroxypropyl)-β-cyclodextrin (HP-β-CD), and about 0.1% cremophor EL (F1).

In an embodiment, the compositions described herein may include, by weight, about 3 to about 4% PS and one or more of about 80% (2-hydroxypropyl)-β-cyclodextrin (HP-β-CD), and about 1% Tween 80 (F2).

In an embodiment, the compositions described herein may include, by weight, about 1% to about 10% PS and one or more of about 1% to about 40% Poloxamer 407 and about 1% to about 20% vitamin E TPGS.

In an embodiment, the compositions described herein may include, by weight, greater than 1% PS and one or more of greater than 1% Poloxamer 407 and greater than 1% vitamin E TPGS.

In some embodiments, the compositions and formulations described herein may include, by w/v % for solid components, and by v/v % for liquid components: about 1.5% PS, about 5% propylene glycol, about 10% mineral oil, about 4% Tween 60, about 4% Tween 80, and about 10% (2-hydroxypropyl)-β-cyclodextrin (HP-β-CD); or about 1.6% PS, about 5% propylene glycol, about 10% mineral oil, about 4% Tween 60, about 4% Tween 80, and about 10% (2-hydroxypropyl)-β-cyclodextrin (HP-β-CD); or about 1.7% PS, about 5% propylene glycol, about 10% mineral oil, about 4% Tween 60, about 4% Tween 80, and about 10% (2-hydroxypropyl)-β-cyclodextrin (HP-β-CD); or about 1.8% PS, about 5% propylene glycol, about 10% mineral oil, about 4% Tween 60, about 4% Tween 80, and about 10% (2-hydroxypropyl)-β-cyclodextrin (HP-β-CD); or about 1.9% PS, about 5% propylene glycol, about 10% mineral oil, about 4% Tween 60, about 4% Tween 80, and about 10% (2-hydroxypropyl)-β-cyclodextrin (HP-β-CD); or about 2% PS, about 5% propylene glycol, about 10% mineral oil, about 4% Tween 60, about 4% Tween 80, about 10% (2-hydroxypropyl)-β-cyclodextrin (HP-β-CD); or about 2.1% PS, about 5% propylene glycol, about 10% mineral oil, about 4% Tween 60, about 4% Tween 80, and about 10% (2-hydroxypropyl)-β-cyclodextrin (HP-β-CD); or about 2.2% PS, about 5% propylene glycol, about 10% mineral oil, about 4% Tween 60, about 4% Tween 80, about 10% (2-hydroxypropyl)-β-cyclodextrin (HP-β-CD); or about 2.3% PS, about 5% propylene glycol, about 10% mineral oil, about 4% Tween 60, about 4% Tween 80, and about 10% (2-hydroxypropyl)-β-cyclodextrin (HP-β-CD); or about 2.4% PS, about 5% propylene glycol, about 10% mineral oil, about 4% Tween 60, about 4% Tween 80, about 10% (2-hydroxypropyl)-β-cyclodextrin (HP-β-CD); or about 2.5% PS, about 5% propylene glycol, about 10% mineral oil, about 4% Tween 60, about 4% Tween 80, and about 10% (2-hydroxypropyl)-β-cyclodextrin (HP-β-CD).

In some embodiments, the compositions and formulations described herein may include, by w/v % for solid components, and by v/v % for liquid components: about 1.5% PS, between about 2.5% and about 7.5% propylene glycol, between about 7.5% and about 12.5% mineral oil, between about 2% and about 6% Tween 60, between about 2% and about 6% Tween 80, and between about 7.5% and about 12.5% (2-hydroxypropyl)-β-cyclodextrin (HP-β-CD); or about 1.6% PS, between about 2.5% and about 7.5% propylene glycol, between about 7.5% and about 12.5% mineral oil, between about 2% and about 6% Tween 60, between about 2% and about 6% Tween 80, and between about 7.5% and about 12.5% (2-hydroxypropyl)-β-cyclodextrin (HP-β-CD); or about 1.7% PS, between about 2.5% and about 7.5% propylene glycol, between about 7.5% and about 12.5% mineral oil, between about 2% and about 6% Tween 60, between about 2% and about 6% Tween 80, and between about 7.5% and about 12.5% (2-hydroxypropyl)-β-cyclodextrin (HP-β-CD); or about 1.8% PS, between about 2.5% and about 7.5% propylene glycol, between about 7.5% and about 12.5% mineral oil, between about 2% and about 6% Tween 60, between about 2% and about 6% Tween 80, and between about 7.5% and about 12.5% (2-hydroxypropyl)-β-cyclodextrin (HP-β-CD); or about 1.9% PS, between about 2.5% and about 7.5% propylene glycol, between about 7.5% and about 12.5% mineral oil, between about 2% and about 6% Tween 60, between about 2% and about 6% Tween 80, and between about 7.5% and about 12.5% (2-hydroxypropyl)-β-cyclodextrin (HP-β-CD); or about 2% PS, between about 2.5% and about 7.5% propylene glycol, between about 7.5% and about 12.5% mineral oil, between about 2% and about 6% Tween 60, between about 2% and about 6% Tween 80, and between about 7.5% and about 12.5% (2-hydroxypropyl)-β-cyclodextrin (HP-β-CD); or about 2.1% PS, between about 2.5% and about 7.5% propylene glycol, between about 7.5% and about 12.5% mineral oil, between about 2% and about 6% Tween 60, between about 2% and about 6% Tween 80, and between about 7.5% and about 12.5% (2-hydroxypropyl)-β-cyclodextrin (HP-β-CD); or about 2.2% PS, between about 2.5% and about 7.5% propylene glycol, between about 7.5% and about 12.5% mineral oil, between about 2% and about 6% Tween 60, between about 2% and about 6% Tween 80, and between about 7.5% and about 12.5% (2-hydroxypropyl)β-cyclodextrin (HP-β-CD); or about 2.3% PS, between about 2.5% and about 7.5% propylene glycol, between about 7.5% and about 12.5% mineral oil, between about 2% and about 6% Tween 60, between about 2% and about 6% Tween 80, and between about 7.5% and about 12.5% (2-hydroxypropyl)β-cyclodextrin (HP-β-CD); or about 2.4% PS, between about 2.5% and about 7.5% propylene glycol, between about 7.5% and about 12.5% mineral oil, between about 2% and about 6% Tween 60, between about 2% and about 6% Tween 80, and between about 7.5% and about 12.5% (2-hydroxypropyl)β-cyclodextrin (HP-β-CD); or about 2.5% PS, between about 2.5% and about 7.5% propylene glycol, between about 7.5% and about 12.5% mineral oil, between about 2% and about 6% Tween 60, between about 2% and about 6% Tween 80, and between about 7.5% and about 12.5% (2-hydroxypropyl)β-cyclodextrin (HP-β-CD).

In some embodiments, the compositions and formulations described herein may include, by w/v % for solid components, and by v/v % for liquid components: between about 0.5% and about 3% PS, between about 18% and about 66% (2-Hydroxypropyl)β-cyclodextrin (HP-β-CD), and between about 1% and about 7% Tween 80; or about 0.5% PS, between about 18% and about 66% (2-Hydroxypropyl)β-cyclodextrin (HP-β-CD), and between about 1% and about 7% Tween 80; or about 0.6% PS, between about 18% and about 66% (2-Hydroxypropyl)β-cyclodextrin (HP-β-CD), and between about 1% and about 7% Tween 80; or about 0.7% PS, between about 18% and about 66% (2-Hydroxypropyl)β-cyclodextrin (HP-β-CD), and between about 1% and about 7% Tween 80; or about 0.8% PS, between about 18% and about 66% (2-Hydroxypropyl)β-cyclodextrin (HP-β-CD), and between about 1% and about 7% Tween 80; or about 0.9% PS, between about 18% and about 66% (2-Hydroxypropyl)β-cyclodextrin (HP-β-CD), and between about 1% and about 7% Tween 80; or about 1% PS, between about 18% and about 66% (2-Hydroxypropyl)β-cyclodextrin (HP-β-CD), and between about 1% and about 7% Tween 80; or about 1.1% PS, between about 18% and about 66% (2-Hydroxypropyl)β-cyclodextrin (HP-β-CD), and between about 1% and about 7% Tween 80; or about 1.2% PS, between about 18% and about 66% (2-Hydroxypropyl)β-cyclodextrin (HP-β-CD), and between about 1% and about 7% Tween 80; or about 1.3% PS, between about 18% and about 66% (2-Hydroxypropyl)β-cyclodextrin (HP-β-CD), and between about 1% and about 7% Tween 80; or about 1.4% PS, between about 18% and about 66% (2-Hydroxypropyl)β-cyclodextrin (HP-β-CD), and between about 1% and about 7% Tween 80; or about 1.5% PS, between about 18% and about 66% (2-Hydroxypropyl)-β-cyclodextrin (HP-β-CD), and between about 1% and about 7% Tween 80; or about 1.6% PS, between about 18% and about 66% (2-Hydroxypropyl)-β-cyclodextrin (HP-β-CD), and between about 1% and about 7% Tween 80; or about 1.7% PS, between about 18% and about 66% (2-Hydroxypropyl)-β-cyclodextrin (HP-β-CD), and between about 1% and about 7% Tween 80; or about 1.8% PS, between about 18% and about 66% (2-Hydroxypropyl)-β-cyclodextrin (HP-β-CD), and between about 1% and about 7% Tween 80; or about 1.9% PS, between about 18% and about 66% (2-Hydroxypropyl)-β-cyclodextrin (HP-β-CD), and between about 1% and about 7% Tween 80; or about 2% PS, between about 18% and about 66% (2-Hydroxypropyl)-β-cyclodextrin (HP-β-CD), and between about 1% and about 7% Tween 80; or about 2.1% PS, between about 18% and about 66% (2-Hydroxypropyl)-β-cyclodextrin (HP-β-CD), and between about 1% and about 7% Tween 80; or about 2.2% PS, between about 18% and about 66% (2-Hydroxypropyl)-β-cyclodextrin (HP-β-CD), and between about 1% and about 7% Tween 80; or about 2.3% PS, between about 18% and about 66% (2-Hydroxypropyl)-β-cyclodextrin (HP-β-CD), and between about 1% and about 7% Tween 80; or about 2.4% PS, between about 18% and about 66% (2-Hydroxypropyl)-β-cyclodextrin (HP-β-CD), and between about 1% and about 7% Tween 80; or about 2.5% PS, between about 18% and about 66% (2-Hydroxypropyl)-β-cyclodextrin (HP-β-CD), and between about 1% and about 7% Tween 80; or about 2.6% PS, between about 18% and about 66% (2-Hydroxypropyl)-β-cyclodextrin (HP-β-CD), and between about 1% and about 7% Tween 80; or about 2.7% PS, between about 18% and about 66% (2-Hydroxypropyl)-β-cyclodextrin (HP-β-CD), and between about 1% and about 7% Tween 80; or about 2.8% PS, between about 18% and about 66% (2-Hydroxypropyl)-β-cyclodextrin (HP-β-CD), and between about 1% and about 7% Tween 80; or about 2.9% PS, between about 18% and about 66% (2-Hydroxypropyl)-β-cyclodextrin (HP-β-CD), and between about 1% and about 7% Tween 80; or about 3% PS, between about 18% and about 66% (2-Hydroxypropyl)-β-cyclodextrin (HP-β-CD), and between about 1% and about 7% Tween 80.

In some embodiments, the compositions and formulations described herein may include, by w/v % for solid components, and by v/v % for liquid components: about 2% PS, about 16% Vitamin E TPGS, about 3.18% mannitol, about 1.2% boric acid, and about 0.005% polyquad; or between about 1% and about 3% PS, between about 10% and about 20% Vitamin E TPGS, between about 1.5% and about 5% mannitol, between about 0.25% and about 2.5% boric acid, and between about 0.001% and about 0.05% polyquad; or about 1% PS, between about 10% and about 20% Vitamin E TPGS, between about 1.5% and about 5% mannitol, between about 0.25% and about 2.5% boric acid, and between about 0.001% and about 0.05% polyquad; or about 1.1% PS, between about 10% and about 20% Vitamin E TPGS, between about 1.5% and about 5% mannitol, between about 0.25% and about 2.5% boric acid, and between about 0.001% and about 0.05% polyquad; or about 1.2% PS, between about 10% and about 20% Vitamin E TPGS, between about 1.5% and about 5% mannitol, between about 0.25% and about 2.5% boric acid, and between about 0.001% and about 0.05% polyquad; or about 1.3% PS, between about 10% and about 20% Vitamin E TPGS, between about 1.5% and about 5% mannitol, between about 0.25% and about 2.5% boric acid, and between about 0.001% and about 0.05% polyquad; or about 1.4% PS, between about 10% and about 20% Vitamin E TPGS, between about 1.5% and about 5% mannitol, between about 0.25% and about 2.5% boric acid, and between about 0.001% and about 0.05% polyquad; or about 1.5% PS, between about 10% and about 20% Vitamin E TPGS, between about 1.5% and about 5% mannitol, between about 0.25% and about 2.5% boric acid, and between about 0.001% and about 0.05% polyquad; or about 1.6% PS, between about 10% and about 20% Vitamin E TPGS, between about 1.5% and about 5% mannitol, between about 0.25% and about 2.5% boric acid, and between about 0.001% and about 0.05% polyquad; or about 1.7% PS, between about 10% and about 20% Vitamin E TPGS, between about 1.5% and about 5% mannitol, between about 0.25% and about 2.5% boric acid, and between about 0.001% and about 0.05% polyquad; or about 1.8% PS, between about 10% and about 20% Vitamin E TPGS, between about 1.5% and about 5% mannitol, between about 0.25% and about 2.5% boric acid, and between about 0.001% and about 0.05% polyquad; or about 1.9% PS, between about 10% and about 20% Vitamin E TPGS, between about 1.5% and about 5% mannitol, between about 0.25% and about 2.5% boric acid, and between about 0.001% and about 0.05% polyquad; or about 2% PS, between about 10% and about 20% Vitamin E TPGS, between about 1.5% and about 5% mannitol, between about 0.25% and about 2.5% boric acid, and between about 0.001% and about 0.05% polyquad; or about 2.1% PS, between about 10% and about 20% Vitamin E TPGS, between about 1.5% and about 5% mannitol, between about 0.25% and about 2.5% boric acid, and between about 0.001% and about 0.05% polyquad; or about 2.2% PS, between about 10% and about 20% Vitamin E TPGS, between about 1.5% and about 5% mannitol, between about 0.25% and about 2.5% boric acid, and between about 0.001% and about 0.05% polyquad; or about 2.3% PS, between about 10% and about 20% Vitamin E TPGS, between about 1.5% and about 5% mannitol, between about 0.25% and about 2.5% boric acid, and between about 0.001% and about 0.05% polyquad; or about 2.4% PS, between about 10% and about 20% Vitamin E TPGS, between about 1.5% and about 5% mannitol, between about 0.25% and about 2.5% boric acid, and between about 0.001% and about 0.05% polyquad; or about 2.5% PS, between about 10% and about 20% Vitamin E TPGS, between about 1.5% and about 5% mannitol, between about 0.25% and about 2.5% boric acid, and between about 0.001% and about 0.05% polyquad; or about 2.6% PS, between about 10% and about 20% Vitamin E TPGS, between about 1.5% and about 5% mannitol, between about 0.25% and about 2.5% boric acid, and between about 0.001% and about 0.05% polyquad; or about 2.7% PS, between about 10% and about 20% Vitamin E TPGS, between about 1.5% and about 5% mannitol, between about 0.25% and about 2.5% boric acid, and between about 0.001% and about 0.05% polyquad; or about 2.8% PS, between about 10% and about 20% Vitamin E TPGS, between about 1.5% and about 5% mannitol, between about 0.25% and about 2.5% boric acid, and between about 0.001% and about 0.05% polyquad; or about 2.9% PS, between about 10% and about 20% Vitamin E TPGS, between about 1.5% and about 5% mannitol, between about 0.25% and about 2.5% boric acid, and between about 0.001% and about 0.05% polyquad; or about 3% PS, between about 10% and about 20% Vitamin E TPGS, between about 1.5% and about 5% mannitol, between about 0.25% and about 2.5% boric acid, and between about 0.001% and about 0.05% polyquad.

In some embodiments, the compositions and formulations described herein may include, by w/v % for solid components, and by v/v % for liquid components: about 0.1% PS, about 10% HP-β-CD, about 4% Tween 80, about 2.5% Vitamin E TPGS, about 1.4% polyvinyl alcohol (PVA) (13,000-26,000 molecular weight), and about 0.001% polyquad. In some embodiments, the compositions and formulations described herein may include, by w/v % for solid components, and by v/v % for liquid components: about 0.01% PS, about 10% HP-β-CD, about 4% Tween 80, about 2.5% Vitamin E TPGS, about 1.4% polyvinyl alcohol (PVA) (13,000-26,000 molecular weight), and about 0.001% polyquad. In some embodiments, the compositions and formulations described herein may include, by w/v % for solid components, and by v/v % for liquid components: about 0.02% PS, about 10% HP-β-CD, about 4% Tween 80, about 2.5% Vitamin E TPGS, about 1.4% polyvinyl alcohol (PVA) (13,000-26,000 molecular weight), and about 0.001% polyquad. In some embodiments, the compositions and formulations described herein may include, by w/v % for solid components, and by v/v % for liquid components: about 0.03% PS, about 10% HP-β-CD, about 4% Tween 80, about 2.5% Vitamin E TPGS, about 1.4% polyvinyl alcohol (PVA) (13,000-26,000 molecular weight), and about 0.001% polyquad. In some embodiments, the compositions and formulations described herein may include, by w/v % for solid components, and by v/v % for liquid components: about 0.04% PS, about 10% HP-β-CD, about 4% Tween 80, about 2.5% Vitamin E TPGS, about 1.4% polyvinyl alcohol (PVA) (13,000-26,000 molecular weight), and about 0.001% polyquad. In some embodiments, the compositions and formulations described herein may include, by w/v % for solid components, and by v/v % for liquid components: about 0.05% PS, about 10% HP-β-CD, about 4% Tween 80, about 2.5% Vitamin E TPGS, about 1.4% polyvinyl alcohol (PVA) (13,000-26,000 molecular weight), and about 0.001% polyquad. In some embodiments, the compositions and formulations described herein may include, by w/v % for solid components, and by v/v % for liquid components: about 0.06% PS, about 10% HP-β-CD, about 4% Tween 80, about 2.5% Vitamin E TPGS, about 1.4% polyvinyl alcohol (PVA) (13,000-26,000 molecular weight), and about 0.001% polyquad. In some embodiments, the compositions and formulations described herein may include, by w/v % for solid components, and by v/v % for liquid components: about 0.07% PS, about 10% HP-β-CD, about 4% Tween 80, about 2.5% Vitamin E TPGS, about 1.4% polyvinyl alcohol (PVA) (13,000-26,000 molecular weight), and about 0.001% polyquad. In some embodiments, the compositions and formulations described herein may include, by w/v % for solid components, and by v/v % for liquid components: about 0.08% PS, about 10% HP-β-CD, about 4% Tween 80, about 2.5% Vitamin E TPGS, about 1.4% polyvinyl alcohol (PVA) (13,000-26,000 molecular weight), and about 0.001% polyquad. In some embodiments, the compositions and formulations described herein may include, by w/v % for solid components, and by v/v % for liquid components: about 0.09% PS, about 10% HP-β-CD, about 4% Tween 80, about 2.5% Vitamin E TPGS, about 1.4% polyvinyl alcohol (PVA) (13,000-26,000 molecular weight), and about 0.001% polyquad. In some embodiments, the compositions and formulations described herein may include, by w/v % for solid components, and by v/v % for liquid components: about 0.1% PS, about 10% HP-β-CD, about 4% Tween 80, about 2.5% Vitamin E TPGS, about 1.4% polyvinyl alcohol (PVA) (13,000-26,000 molecular weight), and about 0.001% polyquad. In some embodiments, the compositions and formulations described herein may include, by w/v % for solid components, and by v/v % for liquid components: about 0.11% PS, about 10% HP-β-CD, about 4% Tween 80, about 2.5% Vitamin E TPGS, about 1.4% polyvinyl alcohol (PVA) (13,000-26,000 molecular weight), and about 0.001% polyquad. In some embodiments, the compositions and formulations described herein may include, by w/v % for solid components, and by v/v % for liquid components: about 0.12% PS, about 10% HP-β-CD, about 4% Tween 80, about 2.5% Vitamin E TPGS, about 1.4% polyvinyl alcohol (PVA) (13,000-26,000 molecular weight), and about 0.001% polyquad. In some embodiments, the compositions and formulations described herein may include, by w/v % for solid components, and by v/v % for liquid components: about 0.13% PS, about 10% HP-β-CD, about 4% Tween 80, about 2.5% Vitamin E TPGS, about 1.4% polyvinyl alcohol (PVA) (13,000-26,000 molecular weight), and about 0.001% polyquad. In some embodiments, the compositions and formulations described herein may include, by w/v % for solid components, and by v/v % for liquid components: about 0.14% PS, about 10% HP-β-CD, about 4% Tween 80, about 2.5% Vitamin E TPGS, about 1.4% polyvinyl alcohol (PVA) (13,000-26,000 molecular weight), and about 0.001% polyquad. In some embodiments, the compositions and formulations described herein may include, by w/v % for solid components, and by v/v % for liquid components: about 0.15% PS, about 10% HP-β-CD, about 4% Tween 80, about 2.5% Vitamin E TPGS, about 1.4% polyvinyl alcohol (PVA) (13,000-26,000 molecular weight), and about 0.001% polyquad. In some embodiments, the compositions and formulations described herein may include, by w/v % for solid components, and by v/v % for liquid components: about 0.16% PS, about 10% HP-β-CD, about 4% Tween 80, about 2.5% Vitamin E TPGS, about 1.4% polyvinyl alcohol (PVA) (13,000-26,000 molecular weight), and about 0.001% polyquad. In some embodiments, the compositions and formulations described herein may include, by w/v % for solid components, and by v/v % for liquid components: about 0.17% PS, about 10% HP-β-CD, about 4% Tween 80, about 2.5% Vitamin E TPGS, about 1.4% polyvinyl alcohol (PVA) (13,000-26,000 molecular weight), and about 0.001% polyquad. In some embodiments, the compositions and formulations described herein may include, by w/v % for solid components, and by v/v % for liquid components: about 0.18% PS, about 10% HP-β-CD, about 4% Tween 80, about 2.5% Vitamin E TPGS, about 1.4% polyvinyl alcohol (PVA) (13,000-26,000 molecular weight), and about 0.001% polyquad. In some embodiments, the compositions and formulations described herein may include, by w/v % for solid components, and by v/v % for liquid components: about 0.19% PS, about 10% HP-β-CD, about 4% Tween 80, about 2.5% Vitamin E TPGS, about 1.4% polyvinyl alcohol (PVA) (13,000-26,000 molecular weight), and about 0.001% polyquad. In some embodiments, the compositions and formulations described herein may include, by w/v % for solid components, and by v/v % for liquid components: about 0.2% PS, about 10% HP-β-CD, about 4% Tween 80, about 2.5% Vitamin E TPGS, about 1.4% polyvinyl alcohol (PVA) (13,000-26,000 molecular weight), and about 0.001% polyquad. In some embodiments, the compositions and formulations described herein may include, by w/v % for solid components, and by v/v % for liquid components: about 0.21% PS, about 10% HP-β-CD, about 4% Tween 80, about 2.5% Vitamin E TPGS, about 1.4% polyvinyl alcohol (PVA) (13,000-26,000 molecular weight), and about 0.001% polyquad. In some embodiments, the compositions and formulations described herein may include, by w/v % for solid components, and by v/v % for liquid components: about 0.22% PS, about 10% HP-β-CD, about 4% Tween 80, about 2.5% Vitamin E TPGS, about 1.4% polyvinyl alcohol (PVA) (13,000-26,000 molecular weight), and about 0.001% polyquad. In some embodiments, the compositions and formulations described herein may include, by w/v % for solid components, and by v/v % for liquid components: about 0.23% PS, about 10% HP-β-CD, about 4% Tween 80, about 2.5% Vitamin E TPGS, about 1.4% polyvinyl alcohol (PVA) (13,000-26,000 molecular weight), and about 0.001% polyquad. In some embodiments, the compositions and formulations described herein may include, by w/v % for solid components, and by v/v % for liquid components: about 0.24% PS, about 10% HP-β-CD, about 4% Tween 80, about 2.5% Vitamin E TPGS, about 1.4% polyvinyl alcohol (PVA) (13,000-26,000 molecular weight), and about 0.001% polyquad. In some embodiments, the compositions and formulations described herein may include, by w/v % for solid components, and by v/v % for liquid components: about 0.25% PS, about 10% HP-β-CD, about 4% Tween 80, about 2.5% Vitamin E TPGS, about 1.4% polyvinyl alcohol (PVA) (13,000-26,000 molecular weight), and about 0.001% polyquad. In some embodiments, the compositions and formulations described herein may include, by w/v % for solid components, and by v/v % for liquid components: about 0.26% PS, about 10% HP-β-CD, about 4% Tween 80, about 2.5% Vitamin E TPGS, about 1.4% polyvinyl alcohol (PVA) (13,000-26,000 molecular weight), and about 0.001% polyquad. In some embodiments, the compositions and formulations described herein may include, by w/v % for solid components, and by v/v % for liquid components: about 0.27% PS, about 10% HP-β-CD, about 4% Tween 80, about 2.5% Vitamin E TPGS, about 1.4% polyvinyl alcohol (PVA) (13,000-26,000 molecular weight), and about 0.001% polyquad. In some embodiments, the compositions and formulations described herein may include, by w/v % for solid components, and by v/v % for liquid components: about 0.28% PS, about 10% HP-β-CD, about 4% Tween 80, about 2.5% Vitamin E TPGS, about 1.4% polyvinyl alcohol (PVA) (13,000-26,000 molecular weight), and about 0.001% polyquad. In some embodiments, the compositions and formulations described herein may include, by w/v % for solid components, and by v/v % for liquid components: about 0.29% PS, about 10% HP-β-CD, about 4% Tween 80, about 2.5% Vitamin E TPGS, about 1.4% polyvinyl alcohol (PVA) (13,000-26,000 molecular weight), and about 0.001% polyquad. In some embodiments, the compositions and formulations described herein may include, by w/v % for solid components, and by v/v % for liquid components: about 0.3% PS, about 10% HP-β-CD, about 4% Tween 80, about 2.5% Vitamin E TPGS, about 1.4% polyvinyl alcohol (PVA) (13,000-26,000 molecular weight), and about 0.001% polyquad.

In some embodiments, the compositions and formulations described herein may include, by w/v % for solid components, and by v/v % for liquid components: about 0.01% PS, between about 7.5% and about 12.5% HP-β-CD, between about 2% and about 6% Tween 80, between about 0.5% and about 5% Vitamin E TPGS, between about 0.25% and about 2.5% polyvinyl alcohol (PVA) (13,000-26,000 molecular weight), and between about 0.0001% and about 0.005% polyquad; or about 0.02% PS, between about 7.5% and about 12.5% HP-β-CD, between about 2% and about 6% Tween 80, between about 0.5% and about 5% Vitamin E TPGS, between about 0.25% and about 2.5% polyvinyl alcohol (PVA) (13,000-26,000 molecular weight), and between about 0.0001% and about 0.005% polyquad; or about 0.03% PS, between about 7.5% and about 12.5% HP-β-CD, between about 2% and about 6% Tween 80, between about 0.5% and about 5% Vitamin E TPGS, between about 0.25% and about 2.5% polyvinyl alcohol (PVA) (13,000-26,000 molecular weight), and between about 0.0001% and about 0.005% polyquad; or about 0.04% PS, between about 7.5% and about 12.5% HP-β-CD, between about 2% and about 6% Tween 80, between about 0.5% and about 5% Vitamin E TPGS, between about 0.25% and about 2.5% polyvinyl alcohol (PVA) (13,000-26,000 molecular weight), and between about 0.0001% and about 0.005% polyquad; or about 0.04% PS, between about 7.5% and about 12.5% HP-β-CD, between about 2% and about 6% Tween 80, between about 0.5% and about 5% Vitamin E TPGS, between about 0.25% and about 2.5% polyvinyl alcohol (PVA) (13,000-26,000 molecular weight), and between about 0.0001% and about 0.005% polyquad; or about 0.05% PS, between about 7.5% and about 12.5% HP-β-CD, between about 2% and about 6% Tween 80, between about 0.5% and about 5% Vitamin E TPGS, between about 0.25% and about 2.5% polyvinyl alcohol (PVA) (13,000-26,000 molecular weight), and between about 0.0001% and about 0.005% polyquad; or about 0.06% PS, between about 7.5% and about 12.5% HP-β-CD, between about 2% and about 6% Tween 80, between about 0.5% and about 5% Vitamin E TPGS, between about 0.25% and about 2.5% polyvinyl alcohol (PVA) (13,000-26,000 molecular weight), and between about 0.0001% and about 0.005% polyquad; or about 0.07% PS, between about 7.5% and about 12.5% HP-β-CD, between about 2% and about 6% Tween 80, between about 0.5% and about 5% Vitamin E TPGS, between about 0.25% and about 2.5% polyvinyl alcohol (PVA) (13,000-26,000 molecular weight), and between about 0.0001% and about 0.005% polyquad; or about 0.08% PS, between about 7.5% and about 12.5% HP-β-CD, between about 2% and about 6% Tween 80, between about 0.5% and about 5% Vitamin E TPGS, between about 0.25% and about 2.5% polyvinyl alcohol (PVA) (13,000-26,000 molecular weight), and between about 0.0001% and about 0.005% polyquad; or about 0.09% PS, between about 7.5% and about 12.5% HP-β-CD, between about 2% and about 6% Tween 80, between about 0.5% and about 5% Vitamin E TPGS, between about 0.25% and about 2.5% polyvinyl alcohol (PVA) (13,000-26,000 molecular weight), and between about 0.0001% and about 0.005% polyquad; or about 0.1% PS, between about 7.5% and about 12.5% HP-β-CD, between about 2% and about 6% Tween 80, between about 0.5% and about 5% Vitamin E TPGS, between about 0.25% and about 2.5% polyvinyl alcohol (PVA) (13,000-26,000 molecular weight), and between about 0.0001% and about 0.005% polyquad; or about 0.11% PS, between about 7.5% and about 12.5% HP-β-CD, between about 2% and about 6% Tween 80, between about 0.5% and about 5% Vitamin E TPGS, between about 0.25% and about 2.5% polyvinyl alcohol (PVA) (13,000-26,000 molecular weight), and between about 0.0001% and about 0.005% polyquad; or about 0.12% PS, between about 7.5% and about 12.5% HP-β-CD, between about 2% and about 6% Tween 80, between about 0.5% and about 5% Vitamin E TPGS, between about 0.25% and about 2.5% polyvinyl alcohol (PVA) (13,000-26,000 molecular weight), and between about 0.0001% and about 0.005% polyquad; or about 0.13% PS, between about 7.5% and about 12.5% HP-β-CD, between about 2% and about 6% Tween 80, between about 0.5% and about 5% Vitamin E TPGS, between about 0.25% and about 2.5% polyvinyl alcohol (PVA) (13,000-26,000 molecular weight), and between about 0.0001% and about 0.005% polyquad; or about 0.14% PS, between about 7.5% and about 12.5% HP-β-CD, between about 2% and about 6% Tween 80, between about 0.5% and about 5% Vitamin E TPGS, between about 0.25% and about 2.5% polyvinyl alcohol (PVA) (13,000-26,000 molecular weight), and between about 0.0001% and about 0.005% polyquad; or about 0.15% PS, between about 7.5% and about 12.5% HP-β-CD, between about 2% and about 6% Tween 80, between about 0.5% and about 5% Vitamin E TPGS, between about 0.25% and about 2.5% polyvinyl alcohol (PVA) (13,000-26,000 molecular weight), and between about 0.0001% and about 0.005% polyquad; or about 0.16% PS, between about 7.5% and about 12.5% HP-β-CD, between about 2% and about 6% Tween 80, between about 0.5% and about 5% Vitamin E TPGS, between about 0.25% and about 2.5% polyvinyl alcohol (PVA) (13,000-26,000 molecular weight), and between about 0.0001% and about 0.005% polyquad; or about 0.17% PS, between about 7.5% and about 12.5% HP-β-CD, between about 2% and about 6% Tween 80, between about 0.5% and about 5% Vitamin E TPGS, between about 0.25% and about 2.5% polyvinyl alcohol (PVA) (13,000-26,000 molecular weight), and between about 0.0001% and about 0.005% polyquad; or about 0.18% PS, between about 7.5% and about 12.5% HP-β-CD, between about 2% and about 6% Tween 80, between about 0.5% and about 5% Vitamin E TPGS, between about 0.25% and about 2.5% polyvinyl alcohol (PVA) (13,000-26,000 molecular weight), and between about 0.0001% and about 0.005% polyquad; or about 0.19% PS, between about 7.5% and about 12.5% HP-β-CD, between about 2% and about 6% Tween 80, between about 0.5% and about 5% Vitamin E TPGS, between about 0.25% and about 2.5% polyvinyl alcohol (PVA) (13,000-26,000 molecular weight), and between about 0.0001% and about 0.005% polyquad; or about 0.2% PS, between about 7.5% and about 12.5% HP-β-CD, between about 2% and about 6% Tween 80, between about 0.5% and about 5% Vitamin E TPGS, between about 0.25% and about 2.5% polyvinyl alcohol (PVA) (13,000-26,000 molecular weight), and between about 0.0001% and about 0.005% polyquad.

In some embodiments, the compositions and formulations described herein may include, by w/v % for solid components, and by v/v % for liquid components: about 4.0% PS, about 20% Poloxamer 407, and about 12% VETPGS (d-α-tocopheryl polyethylene glycol 1000 succinate). In some embodiments, the compositions and formulations described herein may include, by w/v % for solid components, and by v/v % for liquid components: between about 1% and about 7.0% PS, between about 10% and about 30% Poloxamer 407, and between about 5% and about 20% VETPGS (d-α-tocopheryl polyethylene glycol 1000 succinate). In some embodiments, the compositions and formulations described herein may include, by w/v % for solid components, and by v/v % for liquid components: about 4.0% PS, between about 10% and about 30% Poloxamer 407, and between about 5% and about 20% VETPGS (d-α-tocopheryl polyethylene glycol 1000 succinate). In some embodiments, the compositions and formulations described herein may include, by w/v % for solid components, and by v/v % for liquid components: about 0.5% PS, between about 10% and about 30% Poloxamer 407, and between about 5% and about 20% VETPGS (d-α-tocopheryl polyethylene glycol 1000 succinate). In some embodiments, the compositions and formulations described herein may include, by w/v % for solid components, and by v/v % for liquid components: about 1% PS, between about 10% and about 30% Poloxamer 407, and between about 5% and about 20% VETPGS (d-α-tocopheryl polyethylene glycol 1000 succinate). In some embodiments, the compositions and formulations described herein may include, by w/v % for solid components, and by v/v % for liquid components: about 1.5% PS, between about 10% and about 30% Poloxamer 407, and between about 5% and about 20% VETPGS (d-α-tocopheryl polyethylene glycol 1000 succinate). In some embodiments, the compositions and formulations described herein may include, by w/v % for solid components, and by v/v % for liquid components: about 2% PS, between about 10% and about 30% Poloxamer 407, and between about 5% and about 20% VETPGS (d-α-tocopheryl polyethylene glycol 1000 succinate). In some embodiments, the compositions and formulations described herein may include, by w/v % for solid components, and by v/v % for liquid components: about 2.5% PS, between about 10% and about 30% Poloxamer 407, and between about 5% and about 20% VETPGS (d-α-tocopheryl polyethylene glycol 1000 succinate). In some embodiments, the compositions and formulations described herein may include, by w/v % for solid components, and by v/v % for liquid components: about 3% PS, between about 10% and about 30% Poloxamer 407, and between about 5% and about 20% VETPGS (d-α-tocopheryl polyethylene glycol 1000 succinate). In some embodiments, the compositions and formulations described herein may include, by w/v % for solid components, and by v/v % for liquid components: about 3.5% PS, between about 10% and about 30% Poloxamer 407, and between about 5% and about 20% VETPGS (d-α-tocopheryl polyethylene glycol 1000 succinate). In some embodiments, the compositions and formulations described herein may include, by w/v % for solid components, and by v/v % for liquid components: about 4% PS, between about 10% and about 30% Poloxamer 407, and between about 5% and about 20% VETPGS (d-α-tocopheryl polyethylene glycol 1000 succinate). In some embodiments, the compositions and formulations described herein may include, by w/v % for solid components, and by v/v % for liquid components: about 4.5% PS, between about 10% and about 30% Poloxamer 407, and between about 5% and about 20% VETPGS (d-α-tocopheryl polyethylene glycol 1000 succinate). In some embodiments, the compositions and formulations described herein may include, by w/v % for solid components, and by v/v % for liquid components: about 5% PS, between about 10% and about 30% Poloxamer 407, and between about 5% and about 20% VETPGS (d-α-tocopheryl polyethylene glycol 1000 succinate). In some embodiments, the compositions and formulations described herein may include, by w/v % for solid components, and by v/v % for liquid components: about 5.5% PS, between about 10% and about 30% Poloxamer 407, and between about 5% and about 20% VETPGS (d-α-tocopheryl polyethylene glycol 1000 succinate). In some embodiments, the compositions and formulations described herein may include, by w/v % for solid components, and by v/v % for liquid components: about 6% PS, between about 10% and about 30% Poloxamer 407, and between about 5% and about 20% VETPGS (d-α-tocopheryl polyethylene glycol 1000 succinate). In some embodiments, the compositions and formulations described herein may include, by w/v % for solid components, and by v/v % for liquid components: about 6.5% PS, between about 10% and about 30% Poloxamer 407, and between about 5% and about 20% VETPGS (d-α-tocopheryl polyethylene glycol 1000 succinate). In some embodiments, the compositions and formulations described herein may include, by w/v % for solid components, and by v/v % for liquid components: about 7% PS, between about 10% and about 30% Poloxamer 407, and between about 5% and about 20% VETPGS (d-α-tocopheryl polyethylene glycol 1000 succinate). In some embodiments, the compositions and formulations described herein may include, by w/v % for solid components, and by v/v % for liquid components: about 7.5% PS, between about 10% and about 30% Poloxamer 407, and between about 5% and about 20% VETPGS (d-α-tocopheryl polyethylene glycol 1000 succinate). In some embodiments, the compositions and formulations described herein may include, by w/v % for solid components, and by v/v % for liquid components: about 8% PS, between about 10% and about 30% Poloxamer 407, and between about 5% and about 20% VETPGS (d-α-tocopheryl polyethylene glycol 1000 succinate).

In some embodiments, the compositions and formulations described herein may include, by w/v % for solid components, and by v/v % for liquid components: about 0.2% PS, about 4% HP-fβ-CD, about 0.6% Tween 80, about 0.45% Carbopol 980, about 0.2% Vitamin E TPGS, about 0.3% PVA (13,000-26,000 molecular weight), NaCl, and mannitol. In some embodiments, the compositions and formulations described herein may include, by w/v % for solid components, and by v/v % for liquid components: about 0.1% PS, between about 2% and about 6% HP-β-CD, between about 0.1% and about 1.5% Tween 80, between about 0.1% and about 1% Carbopol 980, between about 0.01% and about 0.75% Vitamin E TPGS, between about 0.05% and about 1% PVA (13,000-26,000 molecular weight), NaCl, and mannitol. In some embodiments, the compositions and formulations described herein may include, by w/v % for solid components, and by v/v % for liquid components: about 0.11% PS, between about 2% and about 6% HP-β-CD, between about 0.1% and about 1.5% Tween 80, between about 0.1% and about 1% Carbopol 980, between about 0.01% and about 0.75% Vitamin E TPGS, between about 0.05% and about 1% PVA (13,000-26,000 molecular weight), NaCl, and mannitol. In some embodiments, the compositions and formulations described herein may include, by w/v % for solid components, and by v/v % for liquid components: about 0.12% PS, between about 2% and about 6% HP-β-CD, between about 0.1% and about 1.5% Tween 80, between about 0.1% and about 1% Carbopol 980, between about 0.01% and about 0.75% Vitamin E TPGS, between about 0.05% and about 1% PVA (13,000-26,000 molecular weight), NaCl, and mannitol. In some embodiments, the compositions and formulations described herein may include, by w/v % for solid components, and by v/v % for liquid components: about 0.13% PS, between about 2% and about 6% HP-β-CD, between about 0.1% and about 1.5% Tween 80, between about 0.1% and about 1% Carbopol 980, between about 0.01% and about 0.75% Vitamin E TPGS, between about 0.05% and about 1% PVA (13,000-26,000 molecular weight), NaCl, and mannitol. In some embodiments, the compositions and formulations described herein may include, by w/v % for solid components, and by v/v % for liquid components: about 0.14% PS, between about 2% and about 6% HP-β-CD, between about 0.1% and about 1.5% Tween 80, between about 0.1% and about 1% Carbopol 980, between about 0.01% and about 0.75% Vitamin E TPGS, between about 0.05% and about 1% PVA (13,000-26,000 molecular weight), NaCl, and mannitol. In some embodiments, the compositions and formulations described herein may include, by w/v % for solid components, and by v/v % for liquid components: about 0.15% PS, between about 2% and about 6% HP-β-CD, between about 0.1% and about 1.5% Tween 80, between about 0.1% and about 1% Carbopol 980, between about 0.01% and about 0.75% Vitamin E TPGS, between about 0.05% and about 1% PVA (13,000-26,000 molecular weight), NaCl, and mannitol. In some embodiments, the compositions and formulations described herein may include, by w/v % for solid components, and by v/v % for liquid components: about 0.16% PS, between about 2% and about 6% HP-β-CD, between about 0.1% and about 1.5% Tween 80, between about 0.1% and about 1% Carbopol 980, between about 0.01% and about 0.75% Vitamin E TPGS, between about 0.05% and about 1% PVA (13,000-26,000 molecular weight), NaCl, and mannitol. In some embodiments, the compositions and formulations described herein may include, by w/v % for solid components, and by v/v % for liquid components: about 0.17% PS, between about 2% and about 6% HP-β-CD, between about 0.1% and about 1.5% Tween 80, between about 0.1% and about 1% Carbopol 980, between about 0.01% and about 0.75% Vitamin E TPGS, between about 0.05% and about 1% PVA (13,000-26,000 molecular weight), NaCl, and mannitol. In some embodiments, the compositions and formulations described herein may include, by w/v % for solid components, and by v/v % for liquid components: about 0.18% PS, between about 2% and about 6% HP-β-CD, between about 0.1% and about 1.5% Tween 80, between about 0.1% and about 1% Carbopol 980, between about 0.01% and about 0.75% Vitamin E TPGS, between about 0.05% and about 1% PVA (13,000-26,000 molecular weight), NaCl, and mannitol. In some embodiments, the compositions and formulations described herein may include, by w/v % for solid components, and by v/v % for liquid components: about 0.19% PS, between about 2% and about 6% HP-β-CD, between about 0.1% and about 1.5% Tween 80, between about 0.1% and about 1% Carbopol 980, between about 0.01% and about 0.75% Vitamin E TPGS, between about 0.05% and about 1% PVA (13,000-26,000 molecular weight), NaCl, and mannitol. In some embodiments, the compositions and formulations described herein may include, by w/v % for solid components, and by v/v % for liquid components: about 0.2% PS, between about 2% and about 6% HP-β-CD, between about 0.1% and about 1.5% Tween 80, between about 0.1% and about 1% Carbopol 980, between about 0.01% and about 0.75% Vitamin E TPGS, between about 0.05% and about 1% PVA (13,000-26,000 molecular weight), NaCl, and mannitol. In some embodiments, the compositions and formulations described herein may include, by w/v % for solid components, and by v/v % for liquid components: about 0.21% PS, between about 2% and about 6% HP-β-CD, between about 0.1% and about 1.5% Tween 80, between about 0.1% and about 1% Carbopol 980, between about 0.01% and about 0.75% Vitamin E TPGS, between about 0.05% and about 1% PVA (13,000-26,000 molecular weight), NaCl, and mannitol. In some embodiments, the compositions and formulations described herein may include, by w/v % for solid components, and by v/v % for liquid components: about 0.22% PS, between about 2% and about 6% HP-β-CD, between about 0.1% and about 1.5% Tween 80, between about 0.1% and about 1% Carbopol 980, between about 0.01% and about 0.75% Vitamin E TPGS, between about 0.05% and about 1% PVA (13,000-26,000 molecular weight), NaCl, and mannitol. In some embodiments, the compositions and formulations described herein may include, by w/v % for solid components, and by v/v % for liquid components: about 0.23% PS, between about 2% and about 6% HP-β-CD, between about 0.1% and about 1.5% Tween 80, between about 0.1% and about 1% Carbopol 980, between about 0.01% and about 0.75% Vitamin E TPGS, between about 0.05% and about 1% PVA (13,000-26,000 molecular weight), NaCl, and mannitol. In some embodiments, the compositions and formulations described herein may include, by w/v % for solid components, and by v/v % for liquid components: about 0.24% PS, between about 2% and about 6% HP-β-CD, between about 0.1% and about 1.5% Tween 80, between about 0.1% and about 1% Carbopol 980, between about 0.01% and about 0.75% Vitamin E TPGS, between about 0.05% and about 1% PVA (13,000-26,000 molecular weight), NaCl, and mannitol. In some embodiments, the compositions and formulations described herein may include, by w/v % for solid components, and by v/v % for liquid components: about 0.25% PS, between about 2% and about 6% HP-β-CD, between about 0.1% and about 1.5% Tween 80, between about 0.1% and about 1% Carbopol 980, between about 0.01% and about 0.75% Vitamin E TPGS, between about 0.05% and about 1% PVA (13,000-26,000 molecular weight), NaCl, and mannitol. In some embodiments, the compositions and formulations described herein may include, by w/v % for solid components, and by v/v % for liquid components: about 0.26% PS, between about 2% and about 6% HP-β-CD, between about 0.1% and about 1.5% Tween 80, between about 0.1% and about 1% Carbopol 980, between about 0.01% and about 0.75% Vitamin E TPGS, between about 0.05% and about 1% PVA (13,000-26,000 molecular weight), NaCl, and mannitol. In some embodiments, the compositions and formulations described herein may include, by w/v % for solid components, and by v/v % for liquid components: about 0.27% PS, between about 2% and about 6% HP-β-CD, between about 0.1% and about 1.5% Tween 80, between about 0.1% and about 1% Carbopol 980, between about 0.01% and about 0.75% Vitamin E TPGS, between about 0.05% and about 1% PVA (13,000-26,000 molecular weight), NaCl, and mannitol. In some embodiments, the compositions and formulations described herein may include, by w/v % for solid components, and by v/v % for liquid components: about 0.28% PS, between about 2% and about 6% HP-β-CD, between about 0.1% and about 1.5% Tween 80, between about 0.1% and about 1% Carbopol 980, between about 0.01% and about 0.75% Vitamin E TPGS, between about 0.05% and about 1% PVA (13,000-26,000 molecular weight), NaCl, and mannitol. In some embodiments, the compositions and formulations described herein may include, by w/v % for solid components, and by v/v % for liquid components: about 0.29% PS, between about 2% and about 6% HP-β-CD, between about 0.1% and about 1.5% Tween 80, between about 0.1% and about 1% Carbopol 980, between about 0.01% and about 0.75% Vitamin E TPGS, between about 0.05% and about 1% PVA (13,000-26,000 molecular weight), NaCl, and mannitol. In some embodiments, the compositions and formulations described herein may include, by w/v % for solid components, and by v/v % for liquid components: about 0.3% PS, between about 2% and about 6% HP-β-CD, between about 0.1% and about 1.5% Tween 80, between about 0.1% and about 1% Carbopol 980, between about 0.01% and about 0.75% Vitamin E TPGS, between about 0.05% and about 1% PVA (13,000-26,000 molecular weight), NaCl, and mannitol.

In some embodiments, the compositions and formulations described herein may include, by w/v % for solid components, and by v/v % for liquid components: about 0.6% PS, about 5% HP-β-CD, about 4% Tween 80, about 0.45% Carbopol 980, about 1.25% Vitamin E TPGS, about 0.8% PVA (13,000-26,000 molecular weight), and mannitol (isotonic reagent). In some embodiments, the compositions and formulations described herein may include, by w/v % for solid components, and by v/v % for liquid components: between about 0.1% and about 1.5% PS, between about 2.5% and about 7.5% HP-β-CD, between about 2% and about 6% Tween 80, between about 0.1% and about 1.5% Carbopol 980, between about 0.25% and about 2.25% Vitamin E TPGS, between about 0.1% and about 1.8% PVA (13,000-26,000 molecular weight), and mannitol. In some embodiments, the compositions and formulations described herein may include, by w/v % for solid components, and by v/v % for liquid components: about 0.1% PS, between about 2.5% and about 7.5% HP-β-CD, between about 2% and about 6% Tween 80, between about 0.1% and about 1.5% Carbopol 980, between about 0.25% and about 2.25% Vitamin E TPGS, between about 0.1% and about 1.8% PVA (13,000-26,000 molecular weight), and mannitol; or about 0.2% PS, between about 2.5% and about 7.5% HP-β-CD, between about 2% and about 6% Tween 80, between about 0.1% and about 1.5% Carbopol 980, between about 0.25% and about 2.25% Vitamin E TPGS, between about 0.1% and about 1.8% PVA (13,000-26,000 molecular weight), and mannitol; or about 0.3% PS, between about 2.5% and about 7.5% HP-β-CD, between about 2% and about 6% Tween 80, between about 0.1% and about 1.5% Carbopol 980, between about 0.25% and about 2.25% Vitamin E TPGS, between about 0.1% and about 1.8% PVA (13,000-26,000 molecular weight), and mannitol; or about 0.4% PS, between about 2.5% and about 7.5% HP-β-CD, between about 2% and about 6% Tween 80, between about 0.1% and about 1.5% Carbopol 980, between about 0.25% and about 2.25% Vitamin E TPGS, between about 0.1% and about 1.8% PVA (13,000-26,000 molecular weight), and mannitol; or about 0.5% PS, between about 2.5% and about 7.5% HP-β-CD, between about 2% and about 6% Tween 80, between about 0.1% and about 1.5% Carbopol 980, between about 0.25% and about 2.25% Vitamin E TPGS, between about 0.1% and about 1.8% PVA (13,000-26,000 molecular weight), and mannitol; or about 0.6% PS, between about 2.5% and about 7.5% HP-β-CD, between about 2% and about 6% Tween 80, between about 0.1% and about 1.5% Carbopol 980, between about 0.25% and about 2.25% Vitamin E TPGS, between about 0.1% and about 1.8% PVA (13,000-26,000 molecular weight), and mannitol; or about 0.7% PS, between about 2.5% and about 7.5% HP-β-CD, between about 2% and about 6% Tween 80, between about 0.1% and about 1.5% Carbopol 980, between about 0.25% and about 2.25% Vitamin E TPGS, between about 0.1% and about 1.8% PVA (13,000-26,000 molecular weight), and mannitol; or about 0.8% PS, between about 2.5% and about 7.5% HP-β-CD, between about 2% and about 6% Tween 80, between about 0.1% and about 1.5% Carbopol 980, between about 0.25% and about 2.25% Vitamin E TPGS, between about 0.1% and about 1.8% PVA (13,000-26,000 molecular weight), and mannitol; or about 0.9% PS, between about 2.5% and about 7.5% HP-β-CD, between about 2% and about 6% Tween 80, between about 0.1% and about 1.5% Carbopol 980, between about 0.25% and about 2.25% Vitamin E TPGS, between about 0.1% and about 1.8% PVA (13,000-26,000 molecular weight), and mannitol; or about 1% PS, between about 2.5% and about 7.5% HP-β-CD, between about 2% and about 6% Tween 80, between about 0.1% and about 1.5% Carbopol 980, between about 0.25% and about 2.25% Vitamin E TPGS, between about 0.1% and about 1.8% PVA (13,000-26,000 molecular weight), and mannitol; or about 1.1% PS, between about 2.5% and about 7.5% HP-β-CD, between about 2% and about 6% Tween 80, between about 0.1% and about 1.5% Carbopol 980, between about 0.25% and about 2.25% Vitamin E TPGS, between about 0.1% and about 1.8% PVA (13,000-26,000 molecular weight), and mannitol; or about 1.2% PS, between about 2.5% and about 7.5% HP-β-CD, between about 2% and about 6% Tween 80, between about 0.1% and about 1.5% Carbopol 980, between about 0.25% and about 2.25% Vitamin E TPGS, between about 0.1% and about 1.8% PVA (13,000-26,000 molecular weight), and mannitol; or about 1.3% PS, between about 2.5% and about 7.5% HP-β-CD, between about 2% and about 6% Tween 80, between about 0.1% and about 1.5% Carbopol 980, between about 0.25% and about 2.25% Vitamin E TPGS, between about 0.1% and about 1.8% PVA (13,000-26,000 molecular weight), and mannitol; or about 1.4% PS, between about 2.5% and about 7.5% HP-β-CD, between about 2% and about 6% Tween 80, between about 0.1% and about 1.5% Carbopol 980, between about 0.25% and about 2.25% Vitamin E TPGS, between about 0.1% and about 1.8% PVA (13,000-26,000 molecular weight), and mannitol; or about 1.5% PS, between about 2.5% and about 7.5% HP-β-CD, between about 2% and about 6% Tween 80, between about 0.1% and about 1.5% Carbopol 980, between about 0.25% and about 2.25% Vitamin E TPGS, between about 0.1% and about 1.8% PVA (13,000-26,000 molecular weight), and mannitol.

In some embodiments, the compositions and formulations described herein may include between about 0.1% and about 1.3% (w/v) PS, about 10% (w/v) HP-β-CD, about 4%, or between about 0% and about 20% (v/v) Tween 80, about 2.5% (w/v) Vitamin E TPGS, between about 0% and about 1.4% (w/v) polyvinyl alcohol (PVA) (13000-23000 molecular weight), between about 0% and about 0.5% (w/v) carboxymethylcellulose (low, medium, and/or high viscosity), and about 0.001% (w/v) polyquad (Polyquaternium-1). In some embodiments, the compositions and formulations described herein may include between about 0.1% and about 1.3% (w/v) PS, between about 7.5% and about 12.5% (w/v) HP-β-CD, between about 0% and about 20% (v/v) Tween 80, between about 0.5% and about 5% (w/v) Vitamin E TPGS, between about 0% and about 1.4% (w/v) polyvinyl alcohol (PVA) (13000-23000 molecular weight), between about 0% and about 0.5% (w/v) carboxymethylcellulose (low, medium, and/or high viscosity), and between about 0.0005% and about 0.0015% (w/v) polyquad (Polyquaternium-1). In some embodiments, the compositions and formulations described herein may include about 0.1% (w/v) PS, between about 7.5% and about 12.5% (w/v) HP-β-CD, between about 0% and about 20% (v/v) Tween 80, between about 0.5% and about 5% (w/v) Vitamin E TPGS, between about 0% and about 1.4% (w/v) polyvinyl alcohol (PVA) (13000-23000 molecular weight), between about 0% and about 0.5% (w/v) carboxymethylcellulose (low, medium, and/or high viscosity), and between about 0.0005% and about 0.0015% (w/v) polyquad (Polyquaternium-1). In some embodiments, the compositions and formulations described herein may include about 0.2% (w/v) PS, between about 7.5% and about 12.5% (w/v) HP-β-CD, between about 0% and about 20% (v/v) Tween 80, between about 0.5% and about 5% (w/v) Vitamin E TPGS, between about 0% and about 1.4% (w/v) polyvinyl alcohol (PVA) (13000-23000 molecular weight), between about 0% and about 0.5% (w/v) carboxymethylcellulose (low, medium, and/or high viscosity), and between about 0.0005% and about 0.0015% (w/v) polyquad (Polyquaternium-1). In some embodiments, the compositions and formulations described herein may include about 0.3% (w/v) PS, between about 7.5% and about 12.5% (w/v) HP-β-CD, between about 0% and about 20% (v/v) Tween 80, between about 0.5% and about 5% (w/v) Vitamin E TPGS, between about 0% and about 1.4% (w/v) polyvinyl alcohol (PVA) (13000-23000 molecular weight), between about 0% and about 0.5% (w/v) carboxymethylcellulose (low, medium, and/or high viscosity), and between about 0.0005% and about 0.0015% (w/v) polyquad (Polyquaternium-1). In some embodiments, the compositions and formulations described herein may include about 0.4% (w/v) PS, between about 7.5% and about 12.5% (w/v) HP-β-CD, between about 0% and about 20% (v/v) Tween 80, between about 0.5% and about 5% (w/v) Vitamin E TPGS, between about 0% and about 1.4% (w/v) polyvinyl alcohol (PVA) (13000-23000 molecular weight), between about 0% and about 0.5% (w/v) carboxymethylcellulose (low, medium, and/or high viscosity), and between about 0.0005% and about 0.0015% (w/v) polyquad (Polyquaternium-1). In some embodiments, the compositions and formulations described herein may include about 0.5% (w/v) PS, between about 7.5% and about 12.5% (w/v) HP-β-CD, between about 0% and about 20% (v/v) Tween 80, between about 0.5% and about 5% (w/v) Vitamin E TPGS, between about 0% and about 1.4% (w/v) polyvinyl alcohol (PVA) (13000-23000 molecular weight), between about 0% and about 0.5% (w/v) carboxymethylcellulose (low, medium, and/or high viscosity), and between about 0.0005% and about 0.0015% (w/v) polyquad (Polyquaternium-1). In some embodiments, the compositions and formulations described herein may include about 0.6% (w/v) PS, between about 7.5% and about 12.5% (w/v) HP-β-CD, between about 0% and about 20% (v/v) Tween 80, between about 0.5% and about 5% (w/v) Vitamin E TPGS, between about 0% and about 1.4% (w/v) polyvinyl alcohol (PVA) (13000-23000 molecular weight), between about 0% and about 0.5% (w/v) carboxymethylcellulose (low, medium, and/or high viscosity), and between about 0.0005% and about 0.0015% (w/v) polyquad (Polyquaternium-1). In some embodiments, the compositions and formulations described herein may include about 0.7% (w/v) PS, between about 7.5% and about 12.5% (w/v) HP-β-CD, between about 0% and about 20% (v/v) Tween 80, between about 0.5% and about 5% (w/v) Vitamin E TPGS, between about 0% and about 1.4% (w/v) polyvinyl alcohol (PVA) (13000-23000 molecular weight), between about 0% and about 0.5% (w/v) carboxymethylcellulose (low, medium, and/or high viscosity), and between about 0.0005% and about 0.0015% (w/v) polyquad (Polyquaternium-1). In some embodiments, the compositions and formulations described herein may include about 0.8% (w/v) PS, between about 7.5% and about 12.5% (w/v) HP-β-CD, between about 0% and about 20% (v/v) Tween 80, between about 0.5% and about 5% (w/v) Vitamin E TPGS, between about 0% and about 1.4% (w/v) polyvinyl alcohol (PVA) (13000-23000 molecular weight), between about 0% and about 0.5% (w/v) carboxymethylcellulose (low, medium, and/or high viscosity), and between about 0.0005% and about 0.0015% (w/v) polyquad (Polyquaternium-1). In some embodiments, the compositions and formulations described herein may include about 0.9% (w/v) PS, between about 7.5% and about 12.5% (w/v) HP-β-CD, between about 0% and about 20% (v/v) Tween 80, between about 0.5% and about 5% (w/v) Vitamin E TPGS, between about 0% and about 1.4% (w/v) polyvinyl alcohol (PVA) (13000-23000 molecular weight), between about 0% and about 0.5% (w/v) carboxymethylcellulose (low, medium, and/or high viscosity), and between about 0.0005% and about 0.0015% (w/v) polyquad (Polyquaternium-1). In some embodiments, the compositions and formulations described herein may include about 1% (w/v) PS, between about 7.5% and about 12.5% (w/v) HP-β-CD, between about 0% and about 20% (v/v) Tween 80, between about 0.5% and about 5% (w/v) Vitamin E TPGS, between about 0% and about 1.4% (w/v) polyvinyl alcohol (PVA) (13000-23000 molecular weight), between about 0% and about 0.5% (w/v) carboxymethylcellulose (low, medium, and/or high viscosity), and between about 0.0005% and about 0.0015% (w/v) polyquad (Polyquaternium-1). In some embodiments, the compositions and formulations described herein may include about 1.1% (w/v) PS, between about 7.5% and about 12.5% (w/v) HP-β-CD, between about 0% and about 20% (v/v) Tween 80, between about 0.5% and about 5% (w/v) Vitamin E TPGS, between about 0% and about 1.4% (w/v) polyvinyl alcohol (PVA) (13000-23000 molecular weight), between about 0% and about 0.5% (w/v) carboxymethylcellulose (low, medium, and/or high viscosity), and between about 0.0005% and about 0.0015% (w/v) polyquad (Polyquaternium-1). In some embodiments, the compositions and formulations described herein may include about 1.2% (w/v) PS, between about 7.5% and about 12.5% (w/v) HP-β-CD, between about 0% and about 20% (v/v) Tween 80, between about 0.5% and about 5% (w/v) Vitamin E TPGS, between about 0% and about 1.4% (w/v) polyvinyl alcohol (PVA) (13000-23000 molecular weight), between about 0% and about 0.5% (w/v) carboxymethylcellulose (low, medium, and/or high viscosity), and between about 0.0005% and about 0.0015% (w/v) polyquad (Polyquaternium-1). In some embodiments, the compositions and formulations described herein may include about 1.3% (w/v) PS, between about 7.5% and about 12.5% (w/v) HP-β-CD, between about 0% and about 20% (v/v) Tween 80, between about 0.5% and about 5% (w/v) Vitamin E TPGS, between about 0% and about 1.4% (w/v) polyvinyl alcohol (PVA) (13000-23000 molecular weight), between about 0% and about 0.5% (w/v) carboxymethylcellulose (low, medium, and/or high viscosity), and between about 0.0005% and about 0.0015% (w/v) polyquad (Polyquaternium-1). In some embodiments, the compositions and formulations described herein may include about 1.4% (w/v) PS, between about 7.5% and about 12.5% (w/v) HP-β-CD, between about 0% and about 20% (v/v) Tween 80, between about 0.5% and about 5% (w/v) Vitamin E TPGS, between about 0% and about 1.4% (w/v) polyvinyl alcohol (PVA) (13000-23000 molecular weight), between about 0% and about 0.5% (w/v) carboxymethylcellulose (low, medium, and/or high viscosity), and between about 0.0005% and about 0.0015% (w/v) polyquad (Polyquaternium-1). In some embodiments, the compositions and formulations described herein may include about 1.5% (w/v) PS, between about 7.5% and about 12.5% (w/v) HP-β-CD, between about 0% and about 20% (v/v) Tween 80, between about 0.5% and about 5% (w/v) Vitamin E TPGS, between about 0% and about 1.4% (w/v) polyvinyl alcohol (PVA) (13000-23000 molecular weight), between about 0% and about 0.5% (w/v) carboxymethylcellulose (low, medium, and/or high viscosity), and between about 0.0005% and about 0.0015% (w/v) polyquad (Polyquaternium-1).

In some embodiments, the compositions and formulations described herein may include about 0.1% (w/v) PS, about 10% (w/v) HP-β-CD, about 4% (v/v) Tween 80, about 2.5% (w/v) Vitamin E TPGS, about 1.4% (w/v) polyvinyl alcohol (PVA) (13,000-26,000 molecular weight), about 0.5% (w/v) carboxymethylcellulose (medium viscosity), and about 0.001% (w/v) polyquad (Polyquaternium-1). In some embodiments, the compositions and formulations described herein may include about 0.05% (w/v) PS, about 10% (w/v) HP-β-CD, about 4% (v/v) Tween 80, about 2.5% (w/v) Vitamin E TPGS, about 1.4% (w/v) polyvinyl alcohol (PVA) (13,000-26,000 molecular weight), about 0.5% (w/v) carboxymethylcellulose (medium viscosity), and about 0.001% (w/v) polyquad (Polyquaternium-1). In some embodiments, the compositions and formulations described herein may include about 0.06% (w/v) PS, about 10% (w/v) HP-β-CD, about 4% (v/v) Tween 80, about 2.5% (w/v) Vitamin E TPGS, about 1.4% (w/v) polyvinyl alcohol (PVA) (13,000-26,000 molecular weight), about 0.5% (w/v) carboxymethylcellulose (medium viscosity), and about 0.001% (w/v) polyquad (Polyquaternium-1). In some embodiments, the compositions and formulations described herein may include about 0.07% (w/v) PS, about 10% (w/v) HP-β-CD, about 4% (v/v) Tween 80, about 2.5% (w/v) Vitamin E TPGS, about 1.4% (w/v) polyvinyl alcohol (PVA) (13,000-26,000 molecular weight), about 0.5% (w/v) carboxymethylcellulose (medium viscosity), and about 0.001% (w/v) polyquad (Polyquaternium-1). In some embodiments, the compositions and formulations described herein may include about 0.08% (w/v) PS, about 10% (w/v) HP-β-CD, about 4% (v/v) Tween 80, about 2.5% (w/v) Vitamin E TPGS, about 1.4% (w/v) polyvinyl alcohol (PVA) (13,000-26,000 molecular weight), about 0.5% (w/v) carboxymethylcellulose (medium viscosity), and about 0.001% (w/v) polyquad (Polyquaternium-1). In some embodiments, the compositions and formulations described herein may include about 0.09% (w/v) PS, about 10% (w/v) HP-β-CD, about 4% (v/v) Tween 80, about 2.5% (w/v) Vitamin E TPGS, about 1.4% (w/v) polyvinyl alcohol (PVA) (13,000-26,000 molecular weight), about 0.5% (w/v) carboxymethylcellulose (medium viscosity), and about 0.001% (w/v) polyquad (Polyquaternium-1). In some embodiments, the compositions and formulations described herein may include about 0.1% (w/v) PS, about 10% (w/v) HP-β-CD, about 4% (v/v) Tween 80, about 2.5% (w/v) Vitamin E TPGS, about 1.4% (w/v) polyvinyl alcohol (PVA) (13,000-26,000 molecular weight), about 0.5% (w/v) carboxymethylcellulose (medium viscosity), and about 0.001% (w/v) polyquad (Polyquaternium-1). In some embodiments, the compositions and formulations described herein may include about 0.11% (w/v) PS, about 10% (w/v) HP-β-CD, about 4% (v/v) Tween 80, about 2.5% (w/v) Vitamin E TPGS, about 1.4% (w/v) polyvinyl alcohol (PVA) (13,000-26,000 molecular weight), about 0.5% (w/v) carboxymethylcellulose (medium viscosity), and about 0.001% (w/v) polyquad (Polyquaternium-1). In some embodiments, the compositions and formulations described herein may include about 0.12% (w/v) PS, about 10% (w/v) HP-β-CD, about 4% (v/v) Tween 80, about 2.5% (w/v) Vitamin E TPGS, about 1.4% (w/v) polyvinyl alcohol (PVA) (13,000-26,000 molecular weight), about 0.5% (w/v) carboxymethylcellulose (medium viscosity), and about 0.001% (w/v) polyquad (Polyquaternium-1). In some embodiments, the compositions and formulations described herein may include about 0.13% (w/v) PS, about 10% (w/v) HP-β-CD, about 4% (v/v) Tween 80, about 2.5% (w/v) Vitamin E TPGS, about 1.4% (w/v) polyvinyl alcohol (PVA) (13,000-26,000 molecular weight), about 0.5% (w/v) carboxymethylcellulose (medium viscosity), and about 0.001% (w/v) polyquad (Polyquaternium-1). In some embodiments, the compositions and formulations described herein may include about 0.14% (w/v) PS, about 10% (w/v) HP-β-CD, about 4% (v/v) Tween 80, about 2.5% (w/v) Vitamin E TPGS, about 1.4% (w/v) polyvinyl alcohol (PVA) (13,000-26,000 molecular weight), about 0.5% (w/v) carboxymethylcellulose (medium viscosity), and about 0.001% (w/v) polyquad (Polyquaternium-1). In some embodiments, the compositions and formulations described herein may include about 0.15% (w/v) PS, about 10% (w/v) HP-β-CD, about 4% (v/v) Tween 80, about 2.5% (w/v) Vitamin E TPGS, about 1.4% (w/v) polyvinyl alcohol (PVA) (13,000-26,000 molecular weight), about 0.5% (w/v) carboxymethylcellulose (medium viscosity), and about 0.001% (w/v) polyquad (Polyquaternium-1).

In some embodiments, the compositions and formulations described herein may include about 0.1% (w/v) PS, about 10% (w/v) HP-β-CD, about 4% (v/v) Tween 80, about 2.5% (w/v) Vitamin E TPGS, about 0.5% (w/v) carboxymethylcellulose (medium viscosity), and about 0.001% (w/v) polyquad (Polyquaternium-1). In some embodiments, the compositions and formulations described herein may include about 0.05% (w/v) PS, about 10% (w/v) HP-β-CD, about 4% (v/v) Tween 80, about 2.5% (w/v) Vitamin E TPGS, about 0.5% (w/v) carboxymethylcellulose (medium viscosity), and about 0.001% (w/v) polyquad (Polyquaternium-1). In some embodiments, the compositions and formulations described herein may include about 0.06% (w/v) PS, about 10% (w/v) HP-β-CD, about 4% (v/v) Tween 80, about 2.5% (w/v) Vitamin E TPGS, about 0.5% (w/v) carboxymethylcellulose (medium viscosity), and about 0.001% (w/v) polyquad (Polyquaternium-1). In some embodiments, the compositions and formulations described herein may include about 0.07% (w/v) PS, about 10% (w/v) HP-β-CD, about 4% (v/v) Tween 80, about 2.5% (w/v) Vitamin E TPGS, about 0.5% (w/v) carboxymethylcellulose (medium viscosity), and about 0.001% (w/v) polyquad (Polyquaternium-1). In some embodiments, the compositions and formulations described herein may include about 0.08% (w/v) PS, about 10% (w/v) HP-β-CD, about 4% (v/v) Tween 80, about 2.5% (w/v) Vitamin E TPGS, about 0.5% (w/v) carboxymethylcellulose (medium viscosity), and about 0.001% (w/v) polyquad (Polyquaternium-1). In some embodiments, the compositions and formulations described herein may include about 0.09% (w/v) PS, about 10% (w/v) HP-β-CD, about 4% (v/v) Tween 80, about 2.5% (w/v) Vitamin E TPGS, about 0.5% (w/v) carboxymethylcellulose (medium viscosity), and about 0.001% (w/v) polyquad (Polyquaternium-1). In some embodiments, the compositions and formulations described herein may include about 0.1% (w/v) PS, about 10% (w/v) HP-β-CD, about 4% (v/v) Tween 80, about 2.5% (w/v) Vitamin E TPGS, about 0.5% (w/v) carboxymethylcellulose (medium viscosity), and about 0.001% (w/v) polyquad (Polyquaternium-1). In some embodiments, the compositions and formulations described herein may include about 0.11% (w/v) PS, about 10% (w/v) HP-β-CD, about 4% (v/v) Tween 80, about 2.5% (w/v) Vitamin E TPGS, about 0.5% (w/v) carboxymethylcellulose (medium viscosity), and about 0.001% (w/v) polyquad (Polyquaternium-1). In some embodiments, the compositions and formulations described herein may include about 0.12% (w/v) PS, about 10% (w/v) HP-β-CD, about 4% (v/v) Tween 80, about 2.5% (w/v) Vitamin E TPGS, about 0.5% (w/v) carboxymethylcellulose (medium viscosity), and about 0.001% (w/v) polyquad (Polyquaternium-1). In some embodiments, the compositions and formulations described herein may include about 0.13% (w/v) PS, about 10% (w/v) HP-β-CD, about 4% (v/v) Tween 80, about 2.5% (w/v) Vitamin E TPGS, about 0.5% (w/v) carboxymethylcellulose (medium viscosity), and about 0.001% (w/v) polyquad (Polyquaternium-1). In some embodiments, the compositions and formulations described herein may include about 0.14% (w/v) PS, about 10% (w/v) HP-β-CD, about 4% (v/v) Tween 80, about 2.5% (w/v) Vitamin E TPGS, about 0.5% (w/v) carboxymethylcellulose (medium viscosity), and about 0.001% (w/v) polyquad (Polyquaternium-1). In some embodiments, the compositions and formulations described herein may include about 0.15% (w/v) PS, about 10% (w/v) HP-β-CD, about 4% (v/v) Tween 80, about 2.5% (w/v) Vitamin E TPGS, about 0.5% (w/v) carboxymethylcellulose (medium viscosity), and about 0.001% (w/v) polyquad (Polyquaternium-1).

In some embodiments, the compositions and formulations described herein may include about 0.1% (w/v) PS, about 10% (w/v) HP-β-CD, about 4% (v/v) Tween 80, about 2.5% (w/v) Vitamin E TPGS, about 1.4% (w/v) polyvinyl alcohol (PVA) (13,000-26,000 molecular weight), and about 0.001% (w/v) polyquad (Polyquaternium-1). In some embodiments, the compositions and formulations described herein may include about 0.05% (w/v) PS, about 10% (w/v) HP-β-CD, about 4% (v/v) Tween 80, about 2.5% (w/v) Vitamin E TPGS, about 1.4% (w/v) polyvinyl alcohol (PVA) (13,000-26,000 molecular weight), and about 0.001% (w/v) polyquad (Polyquaternium-1). In some embodiments, the compositions and formulations described herein may include about 0.06% (w/v) PS, about 10% (w/v) HP-β-CD, about 4% (v/v) Tween 80, about 2.5% (w/v) Vitamin E TPGS, about 1.4% (w/v) polyvinyl alcohol (PVA) (13,000-26,000 molecular weight), and about 0.001% (w/v) polyquad (Polyquaternium-1). In some embodiments, the compositions and formulations described herein may include about 0.07% (w/v) PS, about 10% (w/v) HP-β-CD, about 4% (v/v) Tween 80, about 2.5% (w/v) Vitamin E TPGS, about 1.4% (w/v) polyvinyl alcohol (PVA) (13,000-26,000 molecular weight), and about 0.001% (w/v) polyquad (Polyquaternium-1). In some embodiments, the compositions and formulations described herein may include about 0.08% (w/v) PS, about 10% (w/v) HP-β-CD, about 4% (v/v) Tween 80, about 2.5% (w/v) Vitamin E TPGS, about 1.4% (w/v) polyvinyl alcohol (PVA) (13,000-26,000 molecular weight), and about 0.001% (w/v) polyquad (Polyquaternium-1). In some embodiments, the compositions and formulations described herein may include about 0.09% (w/v) PS, about 10% (w/v) HP-β-CD, about 4% (v/v) Tween 80, about 2.5% (w/v) Vitamin E TPGS, about 1.4% (w/v) polyvinyl alcohol (PVA) (13,000-26,000 molecular weight), and about 0.001% (w/v) polyquad (Polyquaternium-1). In some embodiments, the compositions and formulations described herein may include about 0.1% (w/v) PS, about 10% (w/v) HP-β-CD, about 4% (v/v) Tween 80, about 2.5% (w/v) Vitamin E TPGS, about 1.4% (w/v) polyvinyl alcohol (PVA) (13,000-26,000 molecular weight), and about 0.001% (w/v) polyquad (Polyquaternium-1). In some embodiments, the compositions and formulations described herein may include about 0.11% (w/v) PS, about 10% (w/v) HP-β-CD, about 4% (v/v) Tween 80, about 2.5% (w/v) Vitamin E TPGS, about 1.4% (w/v) polyvinyl alcohol (PVA) (13,000-26,000 molecular weight), and about 0.001% (w/v) polyquad (Polyquaternium-1). In some embodiments, the compositions and formulations described herein may include about 0.12% (w/v) PS, about 10% (w/v) HP-β-CD, about 4% (v/v) Tween 80, about 2.5% (w/v) Vitamin E TPGS, about 1.4% (w/v) polyvinyl alcohol (PVA) (13,000-26,000 molecular weight), and about 0.001% (w/v) polyquad (Polyquaternium-1). In some embodiments, the compositions and formulations described herein may include about 0.13% (w/v) PS, about 10% (w/v) HP-β-CD, about 4% (v/v) Tween 80, about 2.5% (w/v) Vitamin E TPGS, about 1.4% (w/v) polyvinyl alcohol (PVA) (13,000-26,000 molecular weight), and about 0.001% (w/v) polyquad (Polyquaternium-1). In some embodiments, the compositions and formulations described herein may include about 0.14% (w/v) PS, about 10% (w/v) HP-β-CD, about 4% (v/v) Tween 80, about 2.5% (w/v) Vitamin E TPGS, about 1.4% (w/v) polyvinyl alcohol (PVA) (13,000-26,000 molecular weight), and about 0.001% (w/v) polyquad (Polyquaternium-1). In some embodiments, the compositions and formulations described herein may include about 0.15% (w/v) PS, about 10% (w/v) HP-β-CD, about 4% (v/v) Tween 80, about 2.5% (w/v) Vitamin E TPGS, about 1.4% (w/v) polyvinyl alcohol (PVA) (13,000-26,000 molecular weight), and about 0.001% (w/v) polyquad (Polyquaternium-1).

In some embodiments, the compositions and formulations described herein may include about 1% PS, about 5% propylene glycol (PG), about 5% Tween 60, about 30% mineral oil, and about 59% petrolatum. In some embodiments, the compositions and formulations described herein may include between about 0.1% and about 2% PS, between about 2.5% and about 7.5% propylene glycol (PG), between about 2.5% and about 7.5% Tween 60, between about 10% and about 50% mineral oil, and between about 25% and about 75% petrolatum. In some embodiments, the compositions and formulations described herein may include: about 0.1% PS, between about 2.5% and about 7.5% propylene glycol (PG), between about 2.5% and about 7.5% Tween 60, between about 10% and about 50% mineral oil, and between about 25% and about 75% petrolatum; or about 0.2% PS, between about 2.5% and about 7.5% propylene glycol (PG), between about 2.5% and about 7.5% Tween 60, between about 10% and about 50% mineral oil, and between about 25% and about 75% petrolatum; or about 0.3% PS, between about 2.5% and about 7.5% propylene glycol (PG), between about 2.5% and about 7.5% Tween 60, between about 10% and about 50% mineral oil, and between about 25% and about 75% petrolatum; or about 0.4% PS, between about 2.5% and about 7.5% propylene glycol (PG), between about 2.5% and about 7.5% Tween 60, between about 10% and about 50% mineral oil, and between about 25% and about 75% petrolatum; or about 0.5% PS, between about 2.5% and about 7.5% propylene glycol (PG), between about 2.5% and about 7.5% Tween 60, between about 10% and about 50% mineral oil, and between about 25% and about 75% petrolatum; or about 0.6% PS, between about 2.5% and about 7.5% propylene glycol (PG), between about 2.5% and about 7.5% Tween 60, between about 10% and about 50% mineral oil, and between about 25% and about 75% petrolatum; or about 0.7% PS, between about 2.5% and about 7.5% propylene glycol (PG), between about 2.5% and about 7.5% Tween 60, between about 10% and about 50% mineral oil, and between about 25% and about 75% petrolatum; or about 0.8% PS, between about 2.5% and about 7.5% propylene glycol (PG), between about 2.5% and about 7.5% Tween 60, between about 10% and about 50% mineral oil, and between about 25% and about 75% petrolatum; or about 0.9% PS, between about 2.5% and about 7.5% propylene glycol (PG), between about 2.5% and about 7.5% Tween 60, between about 10% and about 50% mineral oil, and between about 25% and about 75% petrolatum; or about 1% PS, between about 2.5% and about 7.5% propylene glycol (PG), between about 2.5% and about 7.5% Tween 60, between about 10% and about 50% mineral oil, and between about 25% and about 75% petrolatum; or about 1.1% PS, between about 2.5% and about 7.5% propylene glycol (PG), between about 2.5% and about 7.5% Tween 60, between about 10% and about 50% mineral oil, and between about 25% and about 75% petrolatum; or about 1.2% PS, between about 2.5% and about 7.5% propylene glycol (PG), between about 2.5% and about 7.5% Tween 60, between about 10% and about 50% mineral oil, and between about 25% and about 75% petrolatum; or about 1.3% PS, between about 2.5% and about 7.5% propylene glycol (PG), between about 2.5% and about 7.5% Tween 60, between about 10% and about 50% mineral oil, and between about 25% and about 75% petrolatum; or about 1.4% PS, between about 2.5% and about 7.5% propylene glycol (PG), between about 2.5% and about 7.5% Tween 60, between about 10% and about 50% mineral oil, and between about 25% and about 75% petrolatum; or about 1.5% PS, between about 2.5% and about 7.5% propylene glycol (PG), between about 2.5% and about 7.5% Tween 60, between about 10% and about 50% mineral oil, and between about 25% and about 75% petrolatum; or about 1.6% PS, between about 2.5% and about 7.5% propylene glycol (PG), between about 2.5% and about 7.5% Tween 60, between about 10% and about 50% mineral oil, and between about 25% and about 75% petrolatum; or about 1.7% PS, between about 2.5% and about 7.5% propylene glycol (PG), between about 2.5% and about 7.5% Tween 60, between about 10% and about 50% mineral oil, and between about 25% and about 75% petrolatum; or about 1.8% PS, between about 2.5% and about 7.5% propylene glycol (PG), between about 2.5% and about 7.5% Tween 60, between about 10% and about 50% mineral oil, and between about 25% and about 75% petrolatum; or about 1.9% PS, between about 2.5% and about 7.5% propylene glycol (PG), between about 2.5% and about 7.5% Tween 60, between about 10% and about 50% mineral oil, and between about 25% and about 75% petrolatum; or about 2% PS, between about 2.5% and about 7.5% propylene glycol (PG), between about 2.5% and about 7.5% Tween 60, between about 10% and about 50% mineral oil, and between about 25% and about 75% petrolatum.

In some embodiments, the compositions and formulations described herein may include terpenes and their derivatives such as menthol. In some embodiments, a terpene can be used in any formulation described herein between about 0.025% and about 0.1%, for example about 0.025%, about 0.03%, about 0.035%, about 0.04%, about 0.045%, about 0.05%, about 0.055%, about 0.06%, about 0.065%, about 0.07%, about 0.075%, about 0.08%, about 0.085%, about 0.09%, about 0.095%, or about 0.1%.

In an embodiment, the compositions described herein may include, by weight, less than 10% PS and one or more of less than 40% Poloxamer 407 and less than 20% vitamin E TPGS.

In an embodiment, the compositions described herein may include, by weight, about 5.4% PS and one or more of about 20% Poloxamer 407 and about 12% vitamin E TPGS.

In an embodiment, the compositions described herein may be multicompartment formulations of PS such as, for example, nanoparticles, liposomes, dendrimers, or niosomes that may include PS. Nanoparticles are polymeric carriers, which improve bioavailability thanks to increased corneal penetration and a larger surface area for dissolution. A relative limitation of nanoparticles is their low capacity. Liposomes are limited by their suboptimal stability, high cost and challenging technology for their large-scale production. Niosomes and discosomes are two-layered carriers, which increase API bioavailability by extending its precorneal residence time. In an embodiment, the compositions described herein include nanoparticles that comprise a therapeutically effective amount of PS.

In an embodiment, the compositions described herein may include a nanoparticle formulation comprising a therapeutically effective amount of PS. In some embodiment, the nanoparticle formulation may include poly(ethylene glycol) (PEG) nanoparticles. In some embodiments the nanoparticle formulation may include methoxy poly(ethylene glycol)-poly(lactide) (mPEG-PLA) nanoparticles. In some embodiments, such formulations may allow for delivery of PS to anterior segments of the eye following topical administration. In some embodiments, such formulations may be used to deliver PS to the anterior segments of the eye in an amount sufficient to treat a disease described herein that is associated with such anterior segments of the eye (i.e., a therapeutically effective amount).

In an embodiment, the compositions described herein may include a nanoparticle formulation comprising, by weight, about 1% to about 5% PS and about 90% to about 98% mPEG-PLA.

In an embodiment, the compositions described herein may include a nanoparticle formulation comprising, by weight, about 3% to about 3.5% PS and about 96.5% to about 97% mPEG-PLA.

In certain embodiments, a substantial portion of the total PS that is distributed to the tissues after 1 hour, as determined by HPLC, is in a particular, or targeted, tissue or area. In certain embodiments, greater than 30% of the total PS in the cornea, conjunctiva, aqueous humor, vitreous body, retina, choroid, sclera, lacrimal gland and lens (referred to as tissues or areas of the eye) can be found in a single tissue or area of the eye. In certain embodiments, greater than 30% of the total PS in the cornea, conjunctiva, aqueous humor, vitreous body, retina, choroid, sclera, lacrimal gland and lens can be found in a single tissue or area. In certain embodiments, greater than 40% of the total PS in the cornea, conjunctiva, aqueous humor, vitreous body, retina, choroid, sclera, lacrimal gland and lens can be found in a single tissue or area. In certain embodiments, greater than 50% of the total PS in the cornea, conjunctiva, aqueous humor, vitreous body, retina, choroid, sclera, lacrimal gland and lens can be found in a single tissue or area. In certain embodiments, greater than 60% of the total PS in the cornea, conjunctiva, aqueous humor, vitreous body, retina, choroid, sclera, lacrimal gland and lens can be found in a single tissue or area. In certain embodiments, greater than 70% of the total PS in the cornea, conjunctiva, aqueous humor, vitreous body, retina, choroid, sclera, lacrimal gland and lens can be found in a single tissue or area. In certain embodiments, greater than 80% of the total PS in the cornea, conjunctiva, aqueous humor, vitreous body, retina, choroid, sclera, lacrimal gland and lens can be found in a single tissue or area. In certain embodiments, greater than 90% of the total PS in the cornea, conjunctiva, aqueous humor, vitreous body, retina, choroid, sclera, lacrimal gland and lens can be found in a single tissue or area.

Pharmaceutical Compositions for Injection

In preferred embodiments, the invention provides a pharmaceutical composition for injection, such as intraocular injection, containing a compound of formula (I) or formula (II) described herein, and a pharmaceutical excipient suitable for injection. Components and amounts of compounds in the compositions are as described herein.

The forms in which the compositions of the invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol and liquid polyethylene glycol, such as polyethylene glycol, (and suitable mixtures thereof (e.g., PEG-PLA)), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, and thimerosal.

Sterile injectable solutions are prepared by incorporating a compound of formula (I) or formula (II) described herein in the required amounts in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

### Other Pharmaceutical Compositions

Pharmaceutical compositions may also be prepared from compositions described herein and one or more pharmaceutically acceptable excipients suitable for ocular or intraocular administration. Preparations for such pharmaceutical compositions are well-known in the art. See, e.g., Anderson, et al., eds., Handbook of Clinical Drug Data, Tenth Edition, McGraw-Hill, 2002; and Pratt and Taylor, eds., Principles of Drug Action, Third Edition, Churchill Livingston, N.Y., 1990, each of which is incorporated by reference herein in its entirety.

Administration of a compound of formula (I) or formula (II) described herein or a pharmaceutical composition of these compounds can be effected by any method that enables delivery of the compounds to the site of action. These methods include parenteral injection (including intraocular injection) or topical application (e.g., application to a surface of the eye).

In some embodiments, administration of a compound of formula (I) or formula (II) described herein or a pharmaceutical composition of these compounds can be effected by any method that enables delivery of the compounds to the site of action, which may include oral routes, intraduodenal routes, parenteral injection (including intravenous, intraarterial, subcutaneous, intramuscular, intravascular, intraperitoneal or infusion), topical (e.g., transdermal application, ocular application), rectal administration, via local delivery by catheter or stent or through inhalation. In some embodiments, the compound of formula (I) or formula (II) described herein can also be administered intraadiposally or intrathecally.

Exemplary administration forms (e.g., parenteral, topical, or by drops) include solutions or suspensions of a compound of formula (I) or formula (II) in sterile aqueous solutions, for example, aqueous propylene glycol or dextrose solutions. Such dosage forms can be suitably buffered, if desired.

The invention also provides kits. The kits include a compound of formula (I) or formula (II) described herein in suitable packaging, and written material that can include instructions for use, discussion of clinical studies and listing of side effects. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another active pharmaceutical ingredient (e.g., an antibiotic). In some embodiments, the compound of formula (I) or formula (II) described herein and another active pharmaceutical ingredient are provided as separate compositions in separate containers within the kit. In some embodiments, the compound of formula (I) or formula (II) and the agent are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in some embodiments, be marketed directly to the consumer.

The kits described above are preferably for use in the treatment of the diseases and conditions described herein. In a preferred embodiment, the kits are for use in the treatment of dry eye disease or diabetic retinopathy.

### Dosages and Dosing Regimens

The amounts of a compound of formula (I) or formula (II) described herein administered will be dependent on the human or mammal being treated, the severity of the disorder or condition, the rate of administration, the disposition of the compounds and the discretion of the prescribing physician. However, an effective dosage of each is in the range of about 0.001 to about 100 mg per kg body weight per day, such as about 1 to about 35 mg/kg/day, in single or divided doses. For a 70 kg human, this would amount to about 0.05 to 7 g/day, such as about 0.05 to about 2.5 g/day. In some instances, dosage levels below the lower limit of the aforesaid range may be more than adequate, while in other cases still larger doses may be employed without causing any harmful side effect—e.g., by dividing such larger doses into several small doses for administration throughout the day. The dosage of a compound of formula (I) or formula (II) described herein may be provided in units of mg/kg of body mass or in mg/m² of body surface area.

In some embodiments, a compound of formula (I) or formula (II) described herein is administered in multiple doses. In a preferred embodiment, a compound of formula (I) or formula (II) described herein is administered in multiple doses. Dosing may be once, twice, three times, four times, five times, six times, or more than six times per day. Dosing may be once a month, once every two weeks, once a week, or once every other day. In other embodiments, a compound of formula (I) or formula (II) described herein is administered about once per day to about 6 times per day. In some embodiments, a compound of formula (I) or formula (II) described herein is administered once daily, while in other embodiments, a compound of formula (I) or formula (II) described herein is administered twice daily, and in other embodiments a compound of formula (I) or formula (II) described herein is administered three times daily.

Administration a compound of formula (I) or formula (II) described herein may continue as long as necessary. In some embodiments, a compound of formula (I) or formula (II) described herein is administered for more than 1, 2, 3, 4, 5, 6, 7, 14, or 28 days. In some embodiments, a compound of formula (I) or formula (II) described herein is administered for less than 28, 14, 7, 6, 5, 4, 3, 2, or 1 day. In some embodiments, a compound of formula (I) or formula (II) described herein is administered chronically on an ongoing basis—e.g., for the treatment of chronic effects. In another embodiment, the administration of a compound of formula (I) or formula (II) described herein continues for less than about 7 days. In yet another embodiment, the administration continues for more than about 6, 10, 14, 28 days, two months, six months, or one year. In some cases, continuous dosing is achieved and maintained as long as necessary.

In some embodiments, an effective dosage of a compound of formula (I) or formula (II) described herein is in the range of about 1 mg to about 500 mg, about 10 mg to about 300 mg, about 20 mg to about 250 mg, about 25 mg to about 200 mg, about 10 mg to about 200 mg, about 20 mg to about 150 mg, about 30 mg to about 120 mg, about 10 mg to about 90 mg, about 20 mg to about 80 mg, about 30 mg to about 70 mg, about 40 mg to about 60 mg, about 45 mg to about 55 mg, about 48 mg to about 52 mg, about 50 mg to about 150 mg, about 60 mg to about 140 mg, about 70 mg to about 130 mg, about 80 mg to about 120 mg, about 90 mg to about 110 mg, about 95 mg to about 105 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 202 mg.

In some embodiments, an effective dosage of a compound of formula (I) or formula (II) described herein is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg.

In some instances, dosage levels below the lower limit of the aforesaid ranges may be more than adequate, while in other cases still larger doses may be employed without causing any harmful side effect—e.g., by dividing such larger doses into several small doses for administration throughout the day.

In some embodiments, the compounds described herein are administered topically, e.g., in eye drops. In some embodiments, the therapeutically effective dose for a compound of formula (I) or formula (II) may be at least about 0.75 mg, at least about 1.5 mg, or at least about 2 mg. In some embodiments, the therapeutically effective dose for a compound of formula (I) or formula (II) may be about 0.75 mg, about 1.5 mg, or about 2 mg. In some embodiments, the therapeutically effective dose for a compound of formula (I) or formula (II) is no more than about 0.75 mg, no more than about 1.5 mg, or no more than about 2 mg.

An effective amount of a compound of formula (I) or formula (II) described herein may be administered in either single or multiple doses by any of the accepted modes of administration of agents having similar utilities, including by intraocular injection or topical application.

In some embodiments, the compounds described herein are delivered to mammals for the treatment of disease. A person having ordinary skill in the art would understand that, in certain embodiments, dosages of such compounds may be adjusted depending upon the mammal to be treated. For example, in certain embodiments, the treatment of rabbits is described herein and such dosages may or may not be revised upon the administration of the compounds of the invention to a human. However, a person having ordinary skill in the art may, if necessary, convert the dosages provided herein as set forth in Guidance for Industry: Estimating the Maximum Safe Starting Dose in Initial Clinical Trials for Therapeutics in Adult Healthy Volunteers, U.S. Department of Health and Human Services, Food and Drug Administration, Center for Drug Evaluation and Research (CDER), July 2005, the entirety of which is incorporated herein by reference. In some embodiments, a human equivalent dose (HED) may be determined from an animal dose, the animal dose may be multiplied by the following conversion factors, to provide units in mg/kg: mouse=0.08, hamster=0.13, rat=0.16, ferret=0.19, guinea pig=0.22, rabbit=0.32, dog=0.54, monkey=0.32, marmoset=0.16, squirrel monkey=0.19, baboon=0.54, micro-pig=0.73, and mini-pig=0.95. The foregoing conversion factors are exemplary and in no way limit the dosages provided herein as would be understood by a person having ordinary skill in the art.

While preferred embodiments of the invention are shown and described herein, such embodiments are provided by way of example only and are not intended to otherwise limit the scope of the invention. Various alternatives to the described embodiments of the invention may be employed in practicing the invention.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1—PS as an Efficacious Treatment of Dry Eye Disease in Rabbits

Phospho-sulindac (PS) is a small molecule whose potential clinical applications have been studied. PS is not a prodrug of the NSAID sulindac as the entire PS molecule is required for its pharmacological activity. Here, the potential efficacy of PS in DED is explored.

Various animal models of DED have been reported. In general, mouse models are commonly used in mechanistic studies because of the availability of transgenic strains and relevant antibodies. However, rabbit or dog models are more suitable for the study of dry eye signs and for therapeutic studies, as their eyes are closer to human in size, their ocular surface is easily accessible, and they can have decreased tear production and significant ocular surface changes, recapitulating to a large extent the human disease.

Initially, several DED animal models were experimented with, including benzalkonium and atropine, and their reported limitations were encountered. A clinically relevant short-term rabbit model of DED developed by Nagelhout et al. was focused upon in order to advance drug discovery. In this model, injection of the inferior lacrimal gland (ILG) with the T-cell mitogen Concanavalin A (Con A) led to a pronounced inflammatory process (dacryoadenitis) with elevated levels of MMP-9 and cytokines IL-1β, IL-8, and TGF-β1 in both the lacrimal gland and cornea. The dacryoadenitis suppresses tear production leading to ocular inflammation with attendant changes in clinical parameters of DED. An excellent choice of this model was the use of rabbits, whose eyes, as opposed to those of mice and rats, are closer to the human in size and other features. This model received some validation from reports that anti-inflammatory agents such as dexamethasone reversed clinical manifestations of DED in these rabbits.

Several limitations of this model were observed, mainly lack of reproducibility and the short duration of dry eye (acute model). The former largely stems from the relatively blind injection of Con A into the lacrimal gland, variations in animal anatomy, as well as compensatory tear production from not injected portions of the lacrimal gland system. We have overcome these limitations in our refined model.

The main improvements upon the original Con A-based method brought about our approach are provided herein.

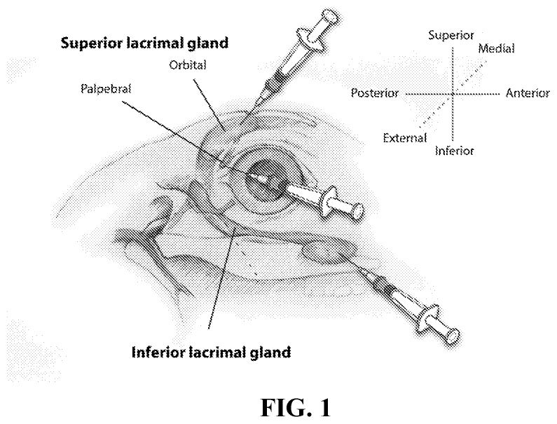

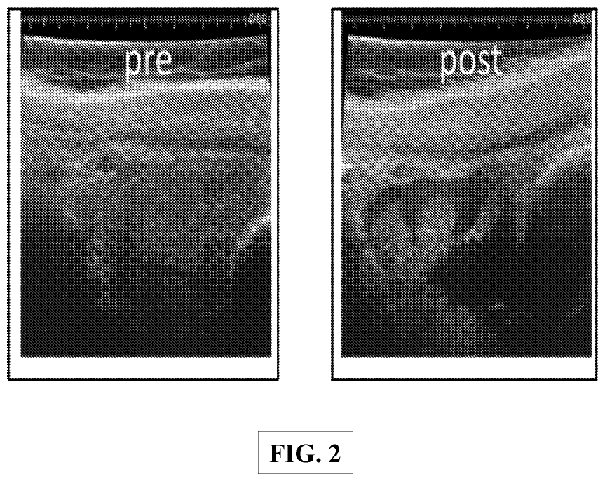

Con A was injected under ultrasound guidance into all the lacrimal glands and the success of the injection was verified by a post-injection ultrasound image (see FIG. 1 and FIG. 2). As observed, the size of the inferior lacrimal glands of rabbits varies 4.1 fold between the smallest and the largest (n=42). This variation explains why the blind injections recommended in the original method are often unsuccessful. This was confirmed by mixing the Con A solution with methylene blue and tracking its course after injection. In about ⅓ of the cases, Con A ended up outside the gland. Rabbits receive three Con A injections, one each into the inferior lacrimal gland (ILG), the palpebral portion of the of the superior lacrimal gland (PSLG), and the orbital portion of the SLG (OSLG).

Injecting all the lacrimal glands and not only the inferior lacrimal gland maximized the suppression of tear production, as it was observed that following the injection of Con A to only one, the remaining lacrimal gland could compensate for dry eye by overproducing tears.

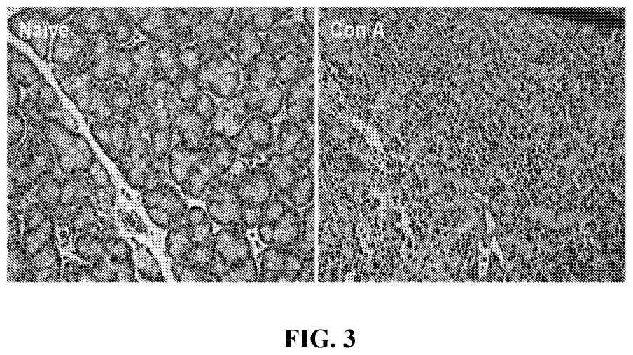

Con A induced a strong inflammatory response in the lacrimal glands characterized by a dense lymphocytic infiltrate (FIG. 3). The inflammation was followed by reduced tear production evidenced by significantly reduced STT values.

Four parameters of efficacy were evaluated instead of the usual one or two. They include (a) the tear break up time (TBUT), determined using 0.2% fluorescein over the eye and recording the time taken to develop black dots, lines or obvious disruption of the fluorescein film; (b) tear osmolarity, measured using TearLab Osmolarity Test and following the manufacturer's instructions (TearLab Corp., San Diego, Calif.); (3) Schirmer tear test (STT), determined using Schirmer strips (EagleVision, Denville, N.J.) inserted between the cornea and the palpebral conjunctiva at the mid-point of the lower lid and measuring the length of moistened strip at 5 min; and (4) tear lactoferrin levels measured by ELISA kit (MyBiosource, San Diego, Calif.) following the instructions of the manufacturer. All four have been used in clinical practice and correlate with the clinical activity of the disease. The STT is the least reliable and, as result, it is clinically used less than half as frequently as TBUT.

The injections of Con A to the lacrimal glands were repeated weekly as needed. When longer than a 1-week periods of study are needed, repeat injections prolong dry eye for at least 3 weeks, making the originally acute model chronic.

This model is robust and can be used to reliably study DED and its response to therapeutic agents.

PS Suppresses Con A—Induced Dry Eye in Rabbits.

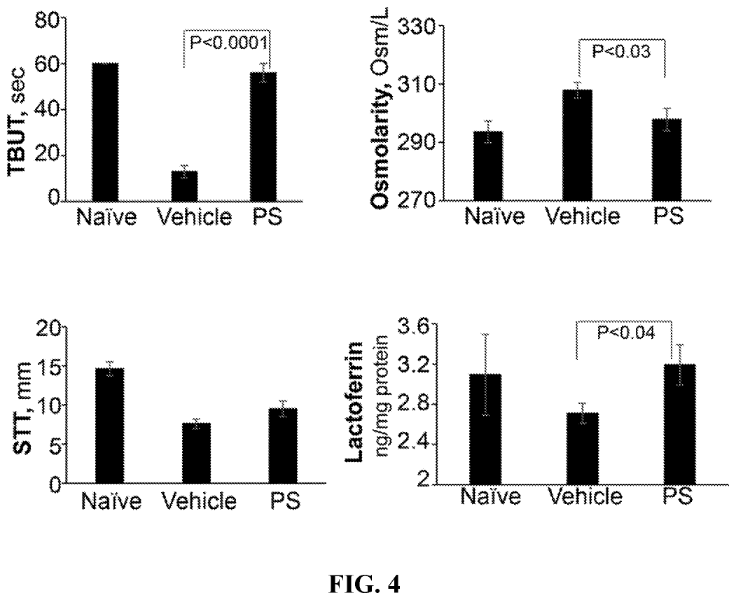

The effect of PS on dry eye was determined in New Zealand White (NZW) rabbits, 2-3 kg (Charles River Labs, Waltham, Mass.). These rabbits were housed singly in rooms with strict temperature (70±5° F.) and humidity (45±5%) control and acclimated for at least 2 weeks prior to induction of dry eye by injection of Con A as above. NZW rabbits with Con A-induced dry eye (three sets of injections) were treated with PS formulated as nanoparticles and administered topically as eye drops 3×/day for 21 days, starting on the day of Con A injection. As shown in FIG. 4, PS restored to normal TBUT, tear osmolarity and tear lactoferrin levels. The STT value also improved but the difference from the vehicle group was significant only for trend. Similar results were obtained on days 5 and 14 (data not shown).

PS is Superior in Efficacy to Cyclosporine and Lifitegrast in DED.

Using this model, we compared the effect of PS to that of cyclosporine and lifitegrast. Rabbits were treated for 6 days with PS as above or cyclosporine 0.05% or lifitegrast 5% eye drops 3×/day. In addition to determining TUBT, osmolarity and STT, we measured the levels of IL-8 and IL-10 in the ILGs of the rabbits harvested at euthanasia. Both of these cytokines are significant mediators of inflammation in DED. As shown in Table 1, PS had statistically significant effects on TBUT, tear osmolarity, IL-8 and IL-1β levels. Cyclosporine improved significantly STT but had no significant effect on the remaining parameters. Lifitegrast improved significantly tear osmolarity but none of the other parameters. Of note, lifitegrast suppressed STT below the levels of the vehicle group and this suppression was statistically significant, but in the opposite direction for a useful therapeutic effect.

TABLE 1
Comparison of PS to Cyclosporine
and Lifitegrast in DED in Rabbits

| | Vehicle | PS | Cyclosporine | Lifitegrast |
|---|---|---|---|---|
| | mean ± SEM | | | |
| TBUT, sec | 12.2 ± 2.8 | 43.6 ± 4.0 | 17 ± 5.4 | 9.1 ± 3.0 |
| | | p < 0.001 | p = 0.11 | p = 0.23 |
| Osmolarity, | 311 ± 2.0 | 294 ± 4.6 | 306 ± 4.1 | 290 ± 4.2 |
| Osm/L | | p < 0.002 | p = 0.22 | p < 0.003 |
| STT, mm | 11.7 ± 1.8 | 12.3 ± 0.6 | 18.3 ± 1.4 | 6.9 ± 0.7 |
| | | | p < 0.01 | p < 0.01* |
| IL-8, | 13.5 ± 5.0 | 4.9 ± 1.7 | 7.4 ± 2.6 | 9.0 ± 2.4 |
| pg/mg protein | | p < 0.05 | p = 0.12 | p = 0.19 |
| IL-1β, | 21.2 ± 6.6 | 8.4 ± 1.2 | 13.5 ± 3.1 | 11.5 ± 1.9 |
| pg/mg protein | | p < 0.03 | p = 0.13 | p = 0.06 |

*This change is in the opposite direction for a useful therapeutic effect.

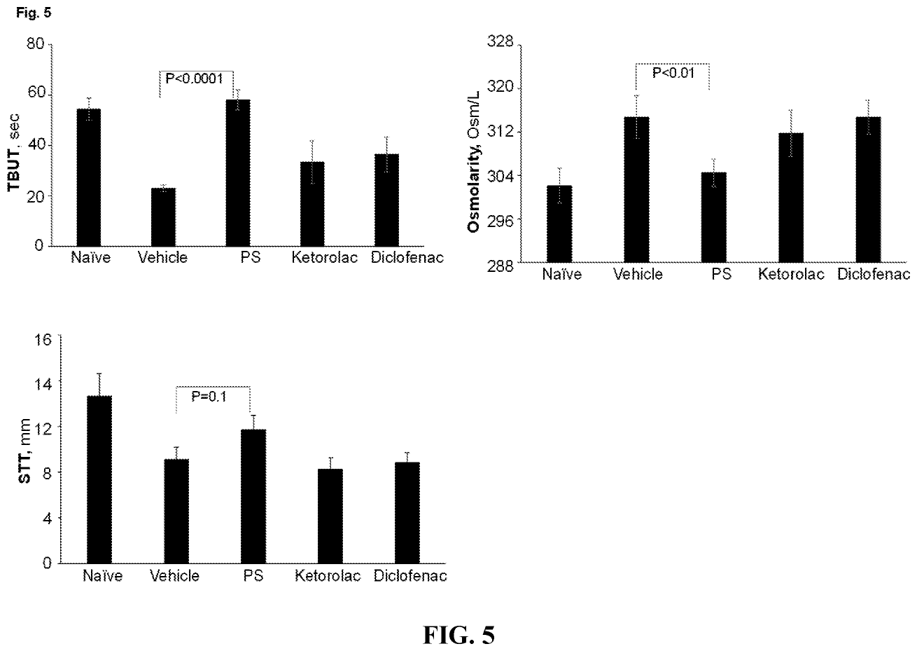

The efficacy of PS on DED was compared to that of ketorolac and diclofenac, two NSAIDs with strong ocular anti-inflammatory and analgesic properties (FIG. 5). After 1 week of treatment, PS as expected normalized TBUT and osmolarity while it had no significant effect on STT. Both ketorolac and diclofenac failed to improve any of these parameters.

The Efficacy of Lower Concentrations of PS.

The efficacy of lower concentrations of PS, 0.1% and 0.2% in DED, was also evaluated. The same animal model (rabbits with Concanavalin A-induced DED) was used. The same methodology described herein was followed, except that PS was administered as two eye drops per eye (˜25 μL each) four times a day. PS was formulated in: 10% (2-hydroxypropyl)-β-cyclodextrin, 4% Tween 80, 2.5% Vitamin E TPGS, 1.4% polyvinyl alcohol (13,000-26,000 molecular weight), 0.001% polyquad, as described herein. The Table below summarizes the corresponding findings:

| | |
|---|---|
| TBUT, sec | STT, mm |

mean ± SEM (n = 12)

| | Baseline | Day 5 | Baseline | Day 5 |
|---|---|---|---|---|
| Vehicle^{a} | 58.2 ± 1.1 | 28.1 ± 5.5 | 14.8 ± 1.2 | 8.2 ± 0.5 |
| PS 0.1%^{b} | 57.9 ± 1.5 | 45.4 ± 5.2 | 16.4 ± 1.5 | 12.0 ± 0.8 |
| PS 0.2%^{c} | 57.9 ± 2.1 | 45.7 ± 4.5 | 15.9 ± 1.0 | 11.8 ± 0.9 |

In this Table, differences are statistically significant only on Day 5 and as follows: For TBUT: a vs. b, p=0.03; a vs. c, p=0.02. For STT: a vs. b, p=0.0004; a vs. c, p=0.002

Both concentrations were very efficacious in treating DED and virtually equipotent. A sharp transition in the dose response of PS was observed regarding several pharmacological effects and these results are an example of this property.

The Safety of Topically Applied PS.

The ocular application of PS was very well tolerated by the rabbits without evidence of discomfort. Slit lamp examination performed weekly during a 1-month application of PS showed no evidence of follicular/papillary response or injection of the conjunctiva nor were there signs of corneal abnormalities (staining defects, corneal vascularization, opacification, epithelial defects, stromal thinning or evidence of melts). Intraocular pressure measured with Tonopen (Reichert Technologies, Depew, N.Y.) remained normal throughout. No animal developed signs of uveitis, and at necropsy the posterior segment appeared normal in all animals.

The Mechanism of Action of PS in Dry Eye.

Tissue culture, animal and human studies have established inflammation as the core mechanism of DED. To determine the mechanism of action of PS in DED the response to PS of several factors known to play an important role in the inflammation associated with DED was explored. They include NF-κB; the cytokines TGF-β, IL-1 β, IL-6 and IL-8; the collagenases MMP-1 and MMP-9; and PGE₂. In these studies human conjunctival epithelial cells were used, the Wong-Kilbourne derivative of Chang conjunctival cells (clone 1 to 5c-4l American Type Culture Collection (Manassas, Va.) certified cell line, 20.2).

PS Suppresses NF-κB Activation.

NF-κB is a transcription factor that modulates a large array of inflammatory mediators and cell signaling cascades, likely playing an important role in the pathogenesis of the ocular inflammation of DED. The effect of PS on NF-κB was evaluated in both cultured human conjunctival cells and in the ILG of rabbits with DED treated with PS or vehicle.

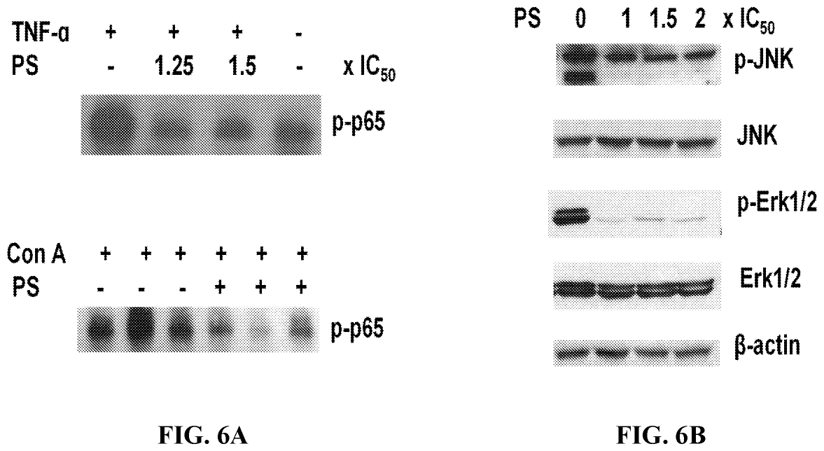

Human conjunctival cells were treated with various concentrations of PS. Five hours later, TNF-α was added to the culture medium to a final concentration of 10 ng/ml and the status of NF-κB activation was determined by EMSA 1 h later. As shown in FIG. 6A, PS significantly suppressed the activation of NF-κB. Similarly, after 1 week of treatment, PS suppressed NF-κB activation in the ILG of rabbits with DED compared to those treated with vehicle.

PS Suppresses MAPK Activation.

MAPKs mediate the response of cells to tear hyperosmolarity and inflammatory cytokines in DED. These kinases can activate the transcription of stress-related genes, including MMP-9. MAPKs stimulate the production of cytokines including IL-β and TNF-α, thereby causing ocular surface damage.

The conjunctiva cells used express only the JNK and Erk½ pathways. PS profoundly suppressed the activation by phosphorylation of both (FIG. 6B).

PS Suppresses Matrix Metalloproteinases (MMPs).

MMPs play a key role in the pathophysiology of DED. MMP-9 (mainly) and MMP-1 and have been implicated in DES. Tear MMP-9 activity parallels the severity of DED. MMPs, e.g., MMP-9, lyse components of the corneal epithelial basement membrane and tight junction proteins. Thus, it was determined that the effect of PS on MMP-1 in cultured conjunctival cells, and on MMP-9 in the ILG, cornea and aqueous humor of rabbits treated with PS.

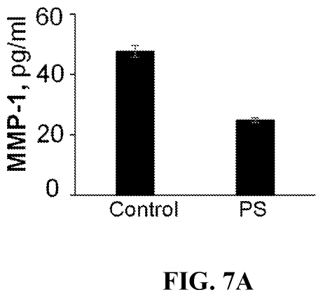

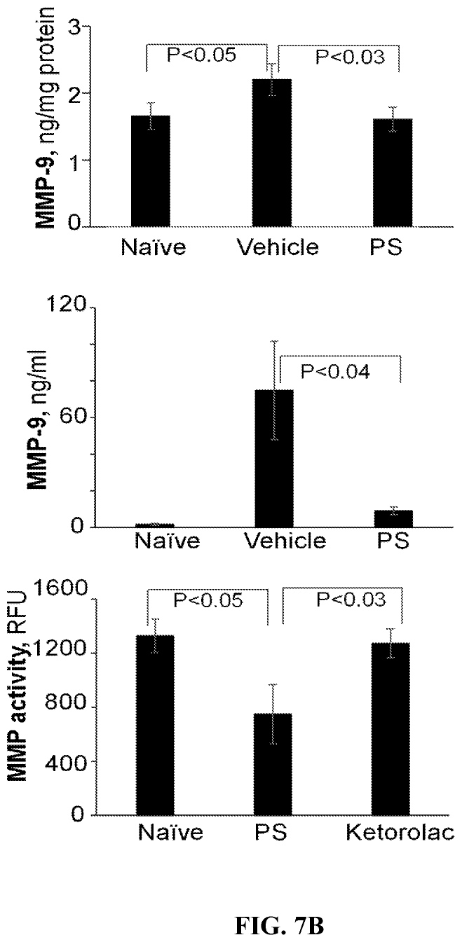

Treatment of cultured human conjunctival cells with PS 1×IC₅₀ or 1.5×IC₅₀ for 2 h, reduced the levels of MMP-1 secreted into the culture medium by 48% and 55%, respectively, compared to controls (47.7±2.0 vs. 24.9±0.8 and 21.6±0.8; mean±SEM; p<0.01 for both; FIG. 7A). These cells did not produce MMP-9. In rabbits treated with Con A the levels of MMP-9 in the ILG and the aqueous humor were significantly increased on day 7 compared to naïve rabbits (no Con A treatment), as shown in FIG. 7B. Treatment of the rabbits having DED with PS for 1 week brought the MMP-9 levels back to normal.

In an acute experiment, naïve rabbits were treated with either PS or ketorolac (both administered topically) for 1 h and determined the activity of MMP in the cornea. This assay determines the activity of MMPs collectively in a given tissue. As shown in FIG. 7B, PS suppressed the activity of MMPs by 43% (p<0.05). In contrast, the NSAID ketorolac failed to affect MMP activity in the cornea.

PS Suppresses Cytokines.

Cytokines play a significant role in DED, with the levels of some of them correlating with individual clinical parameters of DED in humans. It was determined that the response to PS of TGF-β, IL-6, IL-8 and IL-1β in the conjunctival cell line and the ILG of DED rabbits treated with PS.

Cells were treated with PS 1×IC₅₀ and 2 h later TNF-α was added to the medium to a final concentration of 10 ng/ml. Culture media were harvested 24 h later and the levels of TGF-β, IL-6 and IL-8 were determined by ELISA. Of note, the levels of IL-1β were below the limit of detection.

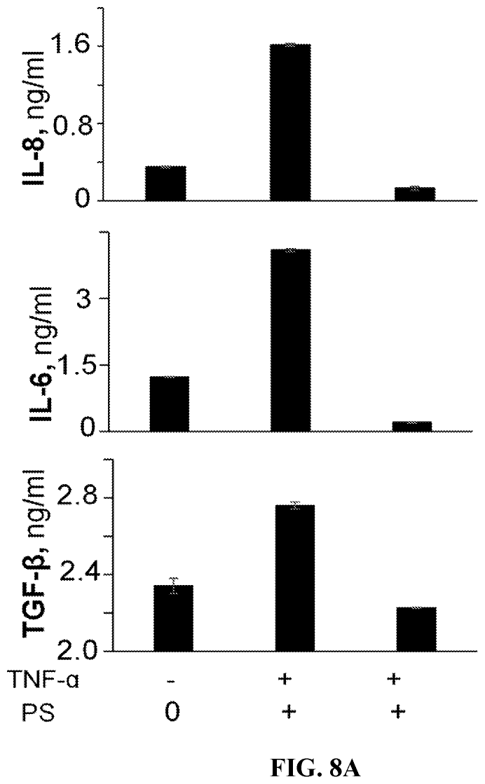

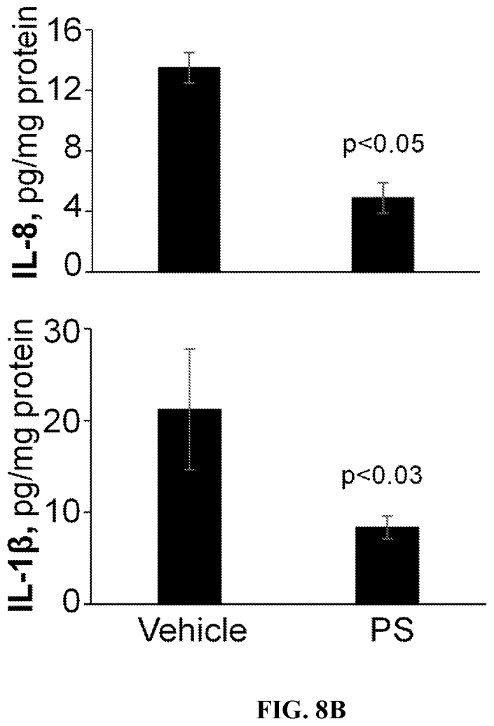

PS markedly suppressed the TNF-α-stimulated levels of IL-8 (92% reduction), IL-6 (95% reduction) and TGF-β (19% reduction) (FIG. 8A). Moreover, for all three cytokines PS suppressed their unstimulated levels as well (62%, 84% and 4.7% reduction, respectively). In addition, PS suppressed the levels of IL-8 by 64% and IL-1β (not expressed by the cultured cells) by 61% in the ILG of rabbits treated with PS for 1 week compared to controls treated with vehicle (FIG. 8B). TGF-β was not detectable by the method in ILG homogenates. All these changes were statistically significant (p<0.001-0.04, except for the unstimulated TGF-β).

PS Preserves the Levels of PGE₂ in Cornea and Tears.

Prostaglandins (PGs) are important inflammatory mediators acting at or near the site of their production. PGE₂ has been implicated in DED, with increased levels of PGE₂ in the tears of patients with DED. Increased COX-2 and PGE synthase expression levels were found in tear-producing tissues of DED mice (no tear levels were reported).

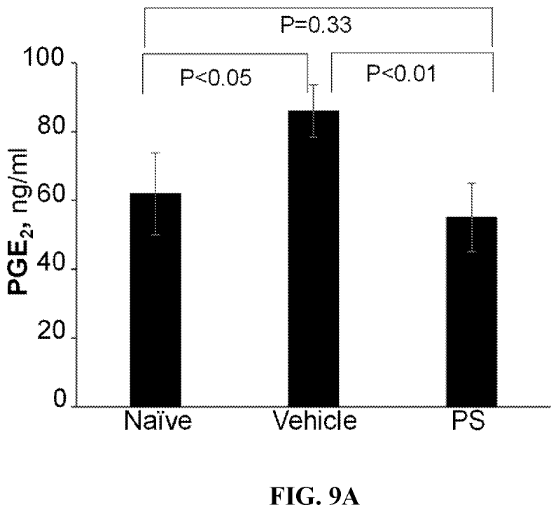

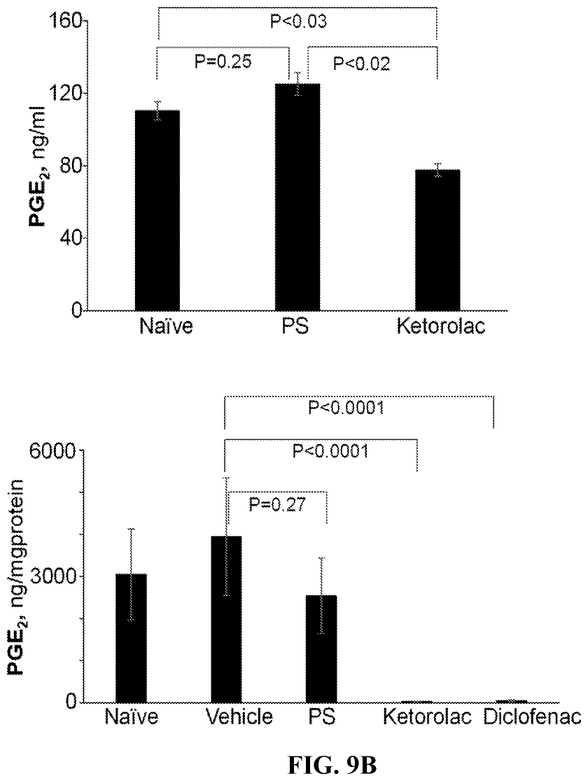

It was determined that the levels of PGE₂ in rabbit tears in three groups of rabbits, naïve and those with Con A-induced DED that were treated for 1 week either with PS or vehicle. As shown in FIGS. 9A and 9B, the tears of vehicle-treated rabbits had significantly higher levels of PGE₂ than naïve rabbits (no Con A, no drug treatment) whereas in PS-treated rabbits these levels were slightly lower than (but not significantly different from) those of naïve rabbits.

In an acute experiment, administered once topically to the eyes of four groups of rabbits with Con A-induced DED was one of the following: vehicle, PS, ketorolac or diclofenac; the latter two are NSAIDs used for the treatment of ocular inflammation and pain. It was determined that the levels of PGE₂ in the cornea of these rabbits obtained 1 h later as well as in the corneas of naïve rabbits. As shown in FIG. 9B, PS that PGE₂ levels in the PS-treated group were no different than those of vehicle-treated and naïve rabbits. This was in sharp contrast to ketorolac and diclofenac, which suppressed nearly completely the levels of PGE₂.

This improved Con A-based model was successfully employed to determine the therapeutic efficacy and safety of a new drug, which demonstrates its applicability to drug development studies and strengthens its validity.

Taken together, these results demonstrate the robust therapeutic effect of PS. PS restored to normal (represented by the naive group) the values of 3 out of the 4 clinical parameters of DED. The only exception was STT, which improved in the PS group, but the change was statistically significant only for trend. Given the serious limitations of this test, however, the STT result does not detract from the conclusion that PS is efficacious.

This conclusion is strengthened by the comparison of the efficacy of PS to that of the two clinically used drugs for DED, cyclosporine and lifitegrast. From a panel of 5 parameters, including two cytokines important in the inflammatory response, IL-1 and IL-8 (the latter correlates with pain in humans), PS induced clinically meaningful responses in 4, as opposed to 1 for each of the other two.

A very important finding has been the absence of any evidence of corneal melt, a feared side effect of NSAID molecules. A defining property of NSAIDs is their ability to inhibit PG synthesis. PS is reported to either inhibit or not affect PGE₂ synthesis. In the cornea and tears, PS preserved the levels of PGE₂. In contrast, ketorolac and diclofenac, two ophthalmic NSAIDs known to induce corneal melt, markedly suppressed PGE₂ levels. It is conceivable that the safety differences between PS and these two NSAIDs could in part be attributed to their different effects on PGE₂. In fact, the cornea of DED is particularly sensitive to NSAIDs, so that they are either contraindicated or should be avoided. A contributor to the development of corneal melt is the activation of MMPs that degrade the collagen stroma of the cornea REF. PS suppressed the levels of MMP9 and the overall activity of MMPs in the cornea. This is in contrast to the lack of such an effect by ketorolac. Without being limited to any one theory of the invention, it appears that the combined effect of PS on PGE₂ and MMP could account for part of the ocular safety of PS. These findings point out a crucial difference between PS and conventional NSAIDs and allow the prediction that corneal melt, not seen during the period of observation, will be an exceedingly unlikely outcome even after long-term administration of PS.

The efficacy of PS in DED appears to result from a constellation of effects on signaling pathways and effector molecules that participate in the pathogenesis of DED. Interestingly, PS displayed significant mechanistic effects on both the surface of the eye and the lacrimal gland, where it reached significant levels. This multi-pathway effect of PS likely explains its strong effect on DED. Inflammation results from the activation of multiple pathways. Thus, suppressing a single pathway even completely may not affect the manifestation of inflammation since the redundancy of the system compensates for the inactivation of one pathway. PS, acting in a multi-targeted manner, avoids such mechanistic resistance, hence its impressive efficacy.

### Example 2—The Ocular and Analgesic Effect of PS

The analgesic effect of PS was examined on the surface of the eye by determining the corneal touch threshold (CTT) using the Luneau Cochet-Bonnet Aesthesiometer (Western Ophthalmics, Lynwood, Wash.) an adjustable nylon monofilament with a defined diameter, which is applied in different lengths to the center of the cornea.

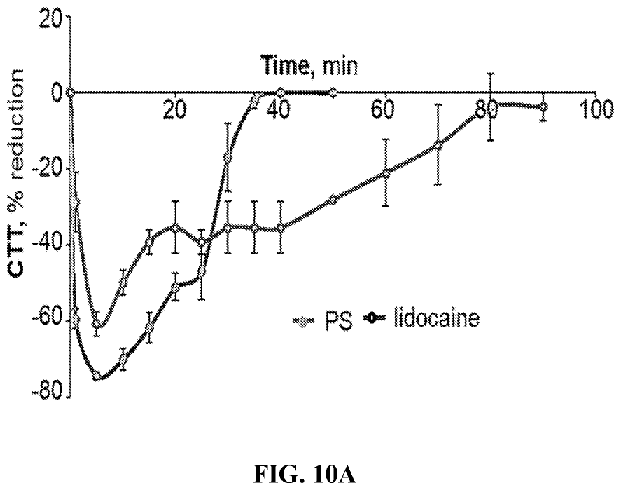

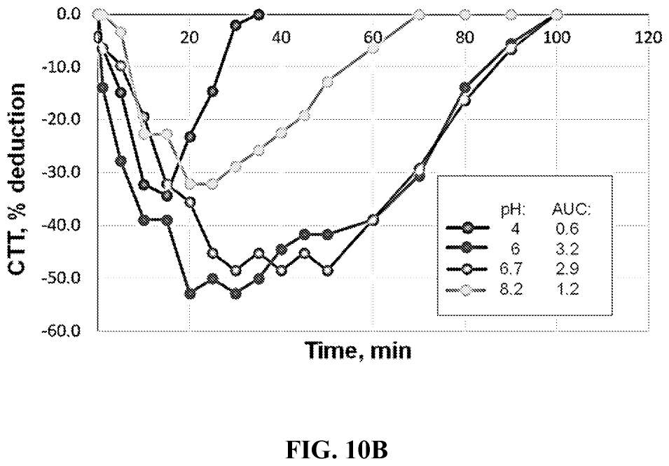

As shown in FIG. 10, PS applied topically to naïve rabbits as a single eye drop produced essentially instantaneous and significant analgesia. Vehicle, used as control, had no effect at all. Lidocaine 1% was the positive control.

Further exploration of the ocular analgesic effect of PS led to the unexpected discovery that both the intensity and duration of this effect can be controlled by controlling the pH of the PS preparation applied onto the ocular surface. FIG. 10B demonstrates that an exemplary cyclodextrin-based formulation of PS in which changes in its pH change the ocular analgesic effect of PS.

In this embodiment, the composition of the PS preparation was: 0.5% PS, 18% (2-hydroxypropyl)-β-cyclodextrin (HP-β-CD), 1-4% Tween 80. Preparation Method: HP-β-CD was dissolved in purified water maintained in a 50° C. water bath. PS was added into this solution and kept at 50° C. overnight with stirring at 500 rpm until PS was fully dissolved. Tween 80 was added into the PS HP-β-CD solution, which was then centrifuged at 3000 rpm for 10 min to remove undissolved particles. The supernatant was collected and pH was adjusted to the desired value using a NaOH solution. The analgesic effect of PS was examined as above.

Further studies of the analgesic effect of PS revealed a totally unexpected and unique property of PS, namely that it acts differently in normal eyes and dry eyes. Patients with DED have decreased corneal sensitivity that appears to be related to damage to corneal sensory innervation (e.g., Burcier T et al; Investigative Ophthalmology and Visual Sciences 2005; 45:2341-2345).

The effect of PS and other compounds on corneal sensitivity was determined in rabbits with normal or dry eyes using the CTT assay as above; DED was induced by Concanavalin A as previously described. The CTT score, expressed as filament length in mm, of normal eyes was 5.56±0.11 mm (mean±SEM for this and subsequent values) and in dry eyes 4.17±0.12 mm; the difference between the two is statistically significant (p<0.0001).

Dry eye disease (DED), considered not to be a single homogeneous disease, includes both dry eye symptoms (sensations of dryness, pain, and visual disturbances) and signs (decreased tear production, increased evaporation, ocular surface inflammation), which are often disparate. Most patients with DED report some degree of ocular pain, which correlates only moderately with the Ocular Surface Disease Index score. In some patients, eyes that feel dry are not dry, while other patients report the perception of dry eye, with burning, irritation and ocular pain that is unresponsive to DED management. Without wishing to be bound by any particular theory, it is believed that PS has a direct analgesic effect on the dry eye, independent of its anti-inflammatory effect. This is evidenced by the immediate (within 5 minutes) response of corneal sensation to it, which lasts less than 100 min, while the functional and anatomical manifestations of DED persist. Without wishing to be bound by any particular theory, it is believed that this analgesic property of PS, not shared by other ocular analgesic drugs or drugs used clinically for the treatment of DED, may be useful to patients with DED whose sensation of dryness and ocular pain persist despite control of DED, in particular its inflammatory component.

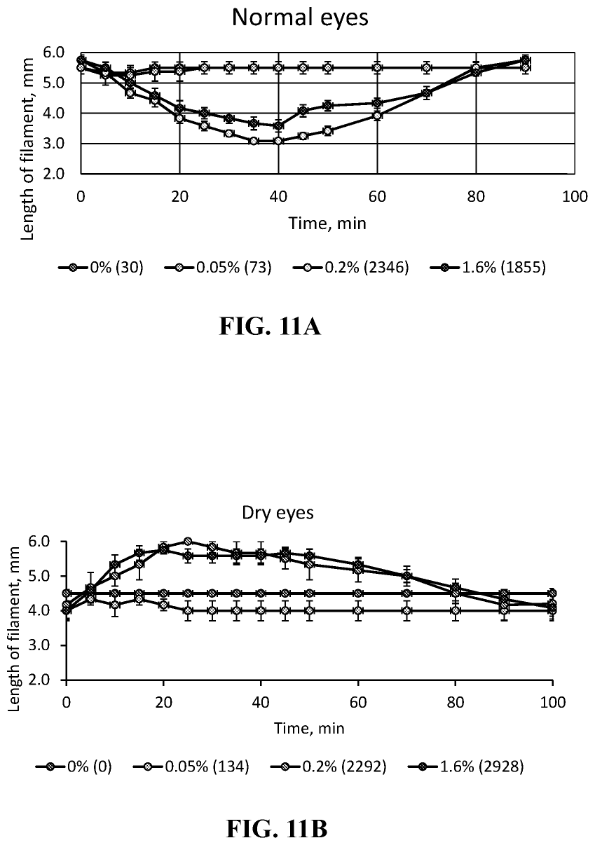

As shown in FIG. 11A, in normal eyes, PS had a dose-dependent analgesic effect. In dry eyes (FIG. 11B), PS restored the already suppressed ocular sensitivity, normalizing it between 15 and 50 min from the time of its application; values progressively returned to baseline, reaching it at 100 min. This effect of PS was detectable 5 min after its application to the cornea, the first time point assayed. There was a clear dose response, with 0.05% PS being ineffective and 0.2% and 1.6% PS being essentially equipotent.

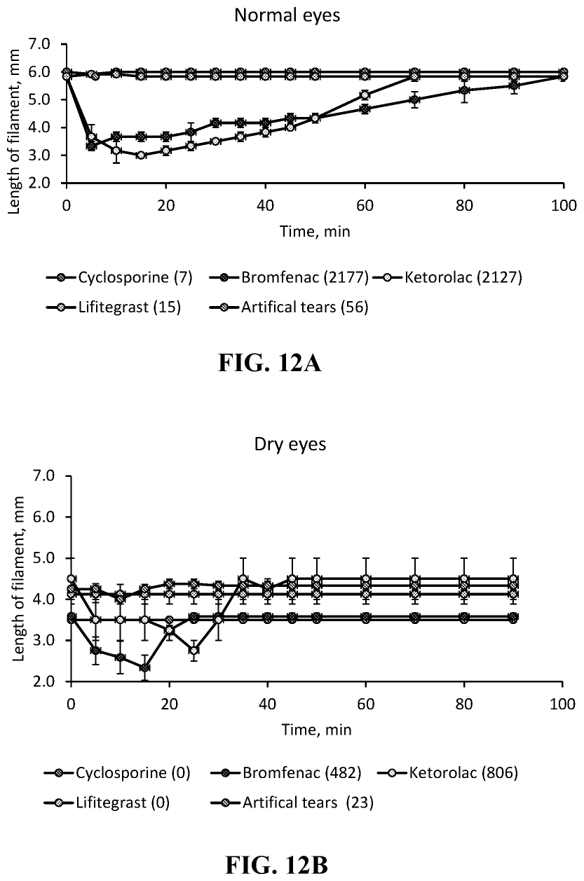

As shown in FIGS. 12A and 12B, only PS possesses the property of restoring an already suppressed ocular sensitivity. Cyclosporin and lifitegrast, both used in the treatment of DED, lack any ocular analgesic effect. Ketorolac and bromfenac, both analgesic/anti-inflammatory ocular agents, display analgesic efficacy in normal eyes, but have no analgesic effect on dry eyes. Artificial tears (Refresh Plus®, carboxymethylcellulose sodium 0.5%), had no effect on CTT scores in either study.

### Example 3—PS Inhibits the Production of VEGF and Neovascularization

Diabetic retinopathy is a disease driven mainly by the formation of new vessels. Inhibiting this process by targeting VEGF, the factor controlling new vessel formation is an established therapeutic strategy. Three sets of experiments demonstrated the ability of PS to inhibit VEGF and new vessel formation.

First, the effect of PS on VEGF production was evaluated by cultured human cancer ovarian cells, known to secrete VEGF to recruit vascular endothelial cells for angiogenesis. Therefore, VEGF is one of the most significant and direct targets in an anti-angiogenesis strategy. The experiments discovered that VEGF levels are reduced in ovarian cancer cells by PS. Secreted VEGF was assayed in the culture medium by ELISA. The results indicated that treatment with PS (1.0×IC₅₀, 24 h) reduced VEGF-A expression levels in both ovarian cancer parental (SKOV3, OVCAR3 and A2780) and resistant variants (A2780cis and A2780ADR). The degree of inhibition ranged between 65% and 100% compared to control as shown in Table 2.

TABLE 2

| | VEGF-A, |
|---|---|
| Cell line | % inhibition |
| SKOV-3 | 96 |
| OVCAR-3 | 100 |
| A2780 | 64 |
| A2780cis | 65 |
| A2780ADR | 77 |

Second, the effect of PS on new vessel formation (neovascularization) was evaluated using the chorioallantoic membrane (CAM) assay. In this assay, fertilized white chicken eggs (SPF Premium, Charles River Laboratory, North Franklin, Conn.) were incubated at 37° C. in 70% humidity for 3 days. The embryos were then incubated ex vivo in a sterile Petri dish for 7 days. Gelatin sponges adsorbed with or without VEGF plus PS or water (vehicle control) were implanted on the CAM surface and the neovasculature was counted on day 4 post implantation under a dissecting microscope.

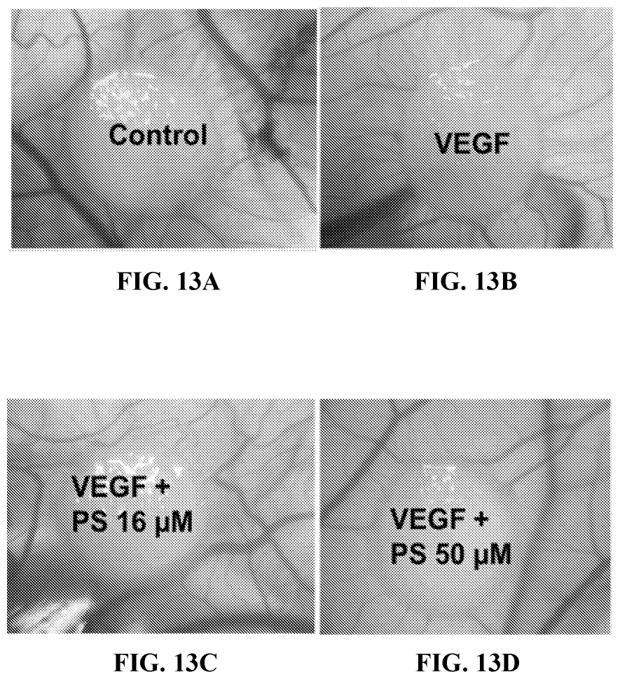

FIG. 13 shows representative images demonstrating the antiangiogenic effect of PS. Table 3 summarizes the associated findings. Within 4 days, PS inhibited neovascularization in CAMs by between 26% and 34% compared to control. The effect was present even when VEGF was not added to the system, as is standard practice

TABLE 3

| | |
|---|---|
| # of new vessels | % inhibition |

Mean ± SEM

| | | |
|---|---|---|
| Control | 58 ± 4.9 | |
| VEGF | 62.3 ± 1.8 | |
| VEGF + PS 16 μM | 46.4 ± 1.5 | 26 (P < 0.0001) |
| VEGF + PS 50 μM | 41.4 ± 1.0 | 34 (P < 0.0001) |
| PS 50 μM | 41 ± 3.2 | 29 (P < 0.016) |

### Example 4: PS Inhibits Oxygen-Induced Retinopathy In Vivo

Several animal models have been explored to understand retinal vascular development. The mouse model of oxygen-induced retinopathy is the most widely used, and has played a pivotal role in our understanding of retinal angiogenesis and in the development of therapeutics such as anti-vascular endothelial growth factor injections for wet age-related macular degeneration. In this model, retinas possess extensive central vaso-obliteration with pathologic neovessels forming around the junction of the vascular and avascular zones, mirroring oxygen-induced retinopathy in humans.

C57BL/6 mice were reared in 75±2% oxygen air starting on postnatal day 7 (P7) and moved into room air on P12, when they were injected intravitreally with 1 μl of 1% PS solution or vehicle. The PS solution consisted of 4.0% PS, 20% Poloxamer 407 and 12% VETPGS (d-α-tocopheryl polyethylene glycol 1000 succinate). On P17, the pups were euthanized, both eyes were enucleated and fixed with 4% paraformaldehyde (PFA). Following several intermediate steps, the retina was existed and fixed further with 4% PFA overnight. After appropriate washings, the retina was incubated with 10 μg/ml of FITC-conjugated anti-lectin antibody overnight and retina flat-mounts were prepared on glass slides and evaluated by fluorescence microscopy. The areas of the avascular, neovascular and whole retina were determined using ImageJ software.

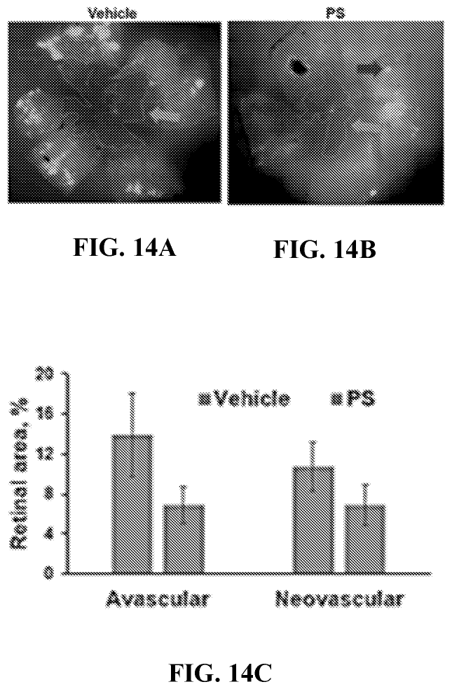

As shown in FIG. 14, compared to vehicle-treated controls, treatment of these mice with PS, reduced the central avascular area by 51% (p<0.04) as well as the peripheral neovascularization (36% inhibition; p<0.07).

### Example 5—PS Topically Applied has a Strong Ocular Anti-Inflammatory Effect

The anti-inflammatory effect of PS in New Zealand white rabbits was evaluated following cataract surgery and administration of the proinflammatory bacterial lipopolysaccharide (LPS). Briefly, the lens was removed by phacoemulsification and aspiration and replaced with the hydrophobic acrylic intraocular lens (AR40e, AMO). Upon completion of the operation, 1 μg of LPS dissolved in 10 μl PBS was injected into the vitreous to induce uveitis.

Rabbits were treated with PS 3.5% formulated in nanoparticles or vehicle (nanoparticles without PS) applied topically as eye drops three times per day. The first application was made within 1 h after completion of surgery. The rabbits were examined daily and the aqueous humor (AH) was sampled by needle aspiration on days 1, 3, and 5 following the injection of LPS. The number of infiltrating cells in the AH was determined following standard methods. On day 5, the rabbits were euthanized and the implanted lens was removed and fixed in 2.5% glutaraldehyde and the number of inflammatory cells attached to the lens was examined under a dissecting microscope.

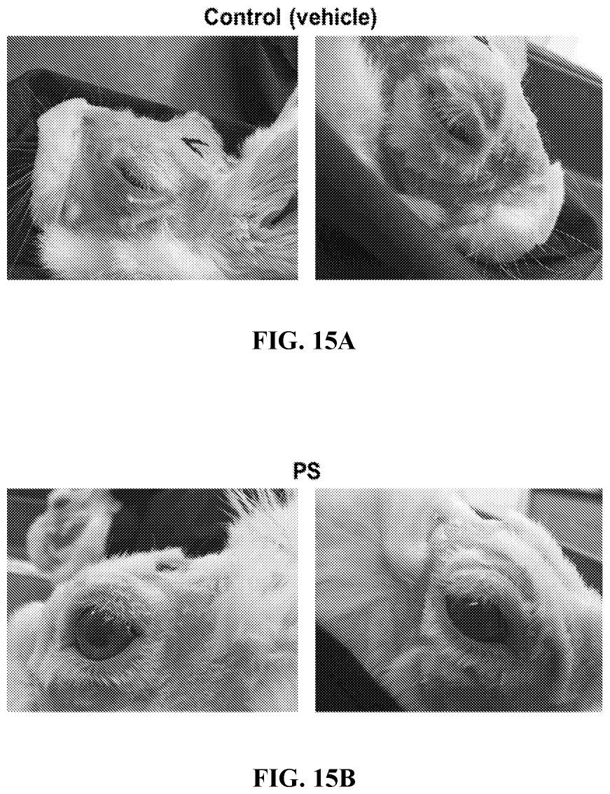

The combination of cataract surgery and LPS injection created a marked inflammatory reaction in the eye and periorbital tissues such that the rabbits were unable to fully open their eyes due to periorbital edema (FIG. 15). Treatment with vehicle failed to improve the ocular inflammation, whereas PS essentially eliminated it during the first 24 h of treatment. The difference in the clinical appearance of the two groups of rabbits (vehicle vs. PS) is dramatic.

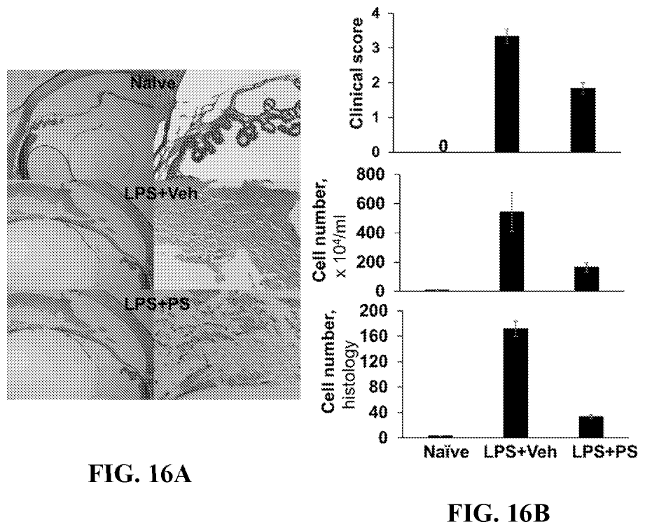

This clinical effect was paralleled by the effect of PS on the number of inflammatory cells in AH. As shown in FIG. 16, on day 3, vehicle-treated rabbits had increased numbers of cells (24-35×10⁴/ml) whereas those treated with PS had <7×10⁴/ml, an effect that paralleled the clinical manifestations of the inflammatory reaction. Similarly, we found that on day 5, when the implanted lenses were removed and examined; those from vehicle-treated rabbits had abundant inflammatory cells attached to them. In contrast, those from PS-treated rabbits had very few or no cells on them (FIG. 16, lower panel).

### Example 6. PS is Efficacious in the Treatment of Uveitis

Uveitis was produced in rats by injecting LPS 75 ng into the footpad of rats. The rats were injected once intravitreally with 2 μPS 3% or vehicle. A control group included naïve rats (not LPS, no treatment). Forty-eight hours later we examined their eyes, sampled the aqueous humor and after euthanizing them we excised, fixed and stained with H&E ocular tissues following standard protocols. As shown in FIG. 14, treatment with PS improved the clinical score (vehicle=3.3±0.2 vs PS=1.8±0.2 (mean±SEM for these and subsequent values); p<0.001), reduced the number of cells (vehicle=543±132 vs PS=164±31; p<0.001); and the inflammatory cells in the tissues of the anterior chamber (vehicle=203±39 vs PS=12±2.3; p<0.001). These findings document a very strong and unexpected therapeutic effect of PS against uveitis.

### Example 7—PS Combined with Antibiotics does not Inhibit Antimicrobial Efficacy

It was assessed whether the combination of PS with antibiotics for their topical application to the eye affects the antimicrobial activity of the antibiotics. To this end, the disk diffusion method was used.

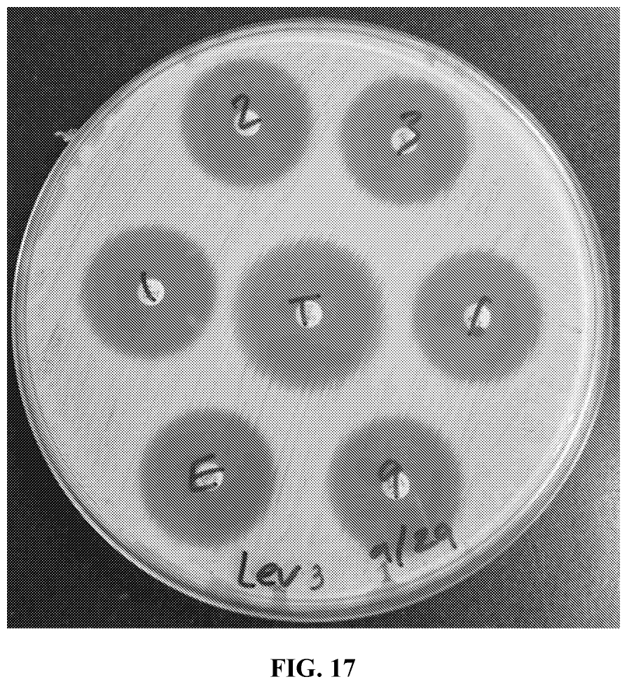

Briefly, Staphylococcus aureus grown in culture was seeded evenly on Muller-Hinton II Agar plates (BD Diagnostic Systems) at the standard concentration of 2×10⁸ colony-forming units per mL. Antibiotic antimicrobial susceptibility disks (Thermo Scientific Oxoid™) were impregnated with one of six concentrations of PS (0%, 1%, 2%, 3%, 6%, 9%); 10 μL of each was evenly dispensed on each disk. An additional control was disks with no PS and no vehicle. The various disks were lightly pressed onto the agar surface as shown in FIG. 17. The growth of bacteria around each disk was monitored and the area of “no growth” around each disk was measured 24 h later.

Results:

As summarized in Table 4 below, PS did not appreciably change the inhibition zone of each antibiotic compared to control (0% PS, i.e., only vehicle). Disks with no PS and no vehicle gave virtually identical results to vehicle controls (not shown). Thus the antimicrobial activity of these two quinolone antibiotics was maintained in the presence of PS even at concentrations significantly exceeding those applied to the eye as eye drops (typically 3%). Similar results were obtained with additional antibiotics.

TABLE 4

| | |
|---|---|
| Ciprofloxacin | Levofloxacin |

| | |
|---|---|
| | Inhibition Zone, mm |
| PS, % | mean ± SD |

| | | |
|---|---|---|
| 0% | 30.0 ± 0.0 | 32.3 ± 0.6 |
| 1% | 30.3 ± 0.6 | 32.7 ± 0.6 |
| 2% | 29.7 ± 0.6 | 32.0 ± 0.0 |
| 3% | 29.3 ± 0.6 | 31.7 ± 0.6 |
| 6% | 27.7 ± 0.4 | 32.3 ± 1.5 |
| 9% | 29.7 ± 0.6 | 31.0 ± 1.0 |

### Example 8—Exemplary PS Formulations that Deliver PS to the Retina

Composition:

3.5% PS; 16% Vitamin E TPGS (d-α-tocopheryl polyethylene glycol 1000 succinate); 3.18% mannitol; 1.2% boric acid; 0.005% polyquaternium-1 (Polyquad). Alternatively, vitamin E TPGS may be replaced by other solubilizing agents. Polyquad is added as a preservative.

Preparation Method:

Polyquad and Vitamin E TPGS were dissolved in purified water followed by addition of PS and stirring at 70° C. for 30 min. Then the solution was centrifuged to remove non-dissolved drug particles and the supernatant was collected, to which mannitol and boric acid were added. The final volume was adjusted with purified water after adjusting the pH to 6.7±0.2 with NaOH.

Results:

The above PS formulation was administered topically as eye drops to the eyes of New Zealand white rabbits. The levels of PS in ocular tissues 1 h and 3 h later were determined by HPLC. Table 5 below summarizes the findings:

TABLE 5

PS, μM

| Tissue | 1 h | 3 h |
|---|---|---|
| Cornea | 6.9 | 0.8 |
| Conjunctiva | 9.3 | 0.5 |
| Aqueous humor | 2.3 | 0.1 |
| Iris | 0.7 | 0.9 |
| Lens | 1.8 | 0.1 |
| Vitreous body | 3.6 | 0.0 |
| Retina | 2.7 | 0.2 |
| Choroid | 3.2 | 0.2 |
| Sclera | 2.3 | 0.2 |
| Lacrimal gland | 0.1 | 0.5 |

### Example 9—Exemplary PS Formulations that Deliver PS to the Anterior Segment of the Eye

Exemplary formulations that allow for delivery of PS exclusively to the anterior segment of the eye are described herein.

A formulation includes 2% PS; 5% Propylene glycol, 10% Mineral oil, 4% Tween 60, 4% Tween 80, 10% (2-hydroxypropyl)-β-cyclodextrin (HP-β-CD). Preparation method (2 mL scale): Oil phase: weight PS into glass vial, add propylene glycol, stir at 50° C. to obtain a clear solution. Then add mineral oil, stir to obtain a clear solution. Water phase: Dissolve HP-β-CD, Kolliphor EL and Tween 80 into water. Add water phase into oil phase, probe-sonicate for 5 sec, 8 times, with 5 sec intervals. Resultant emulsion is filtrated through 0.22 μm filter.

Rabbit ocular pharmacokinetic (PK) study: PS was administered to New Zealand rabbits topically to the eye; as three 25 μL eye drops, 5 min apart. Rabbits were euthanized at eight specified time points between 0.25 to 16 h, ocular tissues were dissected and PS was extracted with acetonitrile and its tissue levels as well as those of its metabolites were determined by HPLC as described (Xie G. et al., Br J Pharmacol 165:2′52-2166; 2012).

The biodistribution of PS was restricted to the anterior chamber; in particular, no PS was detected in the retina. Representative PK parameters shown below established that PS was present at high levels in the cornea and conjunctiva, its AUC0-16h levels dropping below 4 μM·h in the iris and ciliary body.

Ocular PK parameters of PS and its metabolites
after its administration as an emulsion

| | | | | | |
|---|---|---|---|---|---|
| | PS | PS | | sulindac | sulindac |
| PS | sulfide | sulfone | sulindac | sulfone | sulfide |

cornea

| | | | | | | |
|---|---|---|---|---|---|---|
| Cₘₐₓ, μM | 85.4 | 2.7 | 9.7 | 8.7 | 4.0 | 0.0 |
| Tₘₐₓ, h | 0.3 | 0.3 | 1.0 | 1.0 | 4.0 | 0 |
| AUC_{0-16 h}, | 95.0 | 1.0 | 19.7 | 44.9 | 24.4 | 0 |
| μM · h | | | | | | |

conjunctiva

| | | | | | | |
|---|---|---|---|---|---|---|
| Cₘₐₓ, μM | 33.6 | 0.0 | 3.6 | 4.2 | 0.8 | 0.0 |
| Tₘₐₓ, h | 0.3 | 0.0 | 0.3 | 0.3 | 2.0 | 0.0 |
| AUC_{0-16 h}, | 23.7 | 0.0 | 3.1 | 6.1 | 3.2 | 0.0 |
| μM · h | | | | | | |

aqueous humor

| | | | | | | |
|---|---|---|---|---|---|---|
| Cₘₐₓ, μM | 0.9 | 0.0 | 0.0 | 1.0 | 0.0 | 2.0 |
| Tₘₐₓ, h | 0.3 | 0.0 | 0.0 | 1.0 | 0.0 | 2.0 |
| AUC_{0-16 h}, | 1.0 | 0.0 | 0.0 | 1.8 | 0.0 | 0.6 |
| μM · h | | | | | | |

Another formulation includes PS 0.5%˜3%, (2-Hydroxypropyl)β-cyclodextrin (HP-β-CD) 18%˜66%, Tween 80 4%. Preparation Method: HP-β-CD and Tween 80 were dissolved in water; PS was added into above solution, and stirred at 50° C. until PS was fully dissolved. The pH of the solution was adjusted to the required value.

### Gellan Gum-Based In-Situ Gel Formulation

Composition:

2.4-3% PS; 0.5% Gellan gum; 5% Vitamin E TPGS; 10% (2-hydroxypropyl)-β-cyclodextrin. Preparation Method: A Gellan gum solution was prepared by adding a certain amount of gellan gum to deionized water and heating the mixture to 90° C. with fast stirring (500 rpm). Once completely dissolved, the solution was filtered through a 0.22 μm filter. Then, PS and additional excipients were added to the system to achieve the above concentrations and stirred at 50° C. at 500 rpm for 30 minutes to allow complete dissolution.

Results:

The above PS formulation was administered topically as eye drops to the eyes of New Zealand white rabbits. The levels of PS in ocular tissues at 2 h later were determined by HPLC. Table 6 summarizes the findings.

TABLE 6

| Tissue | PS, μM at 2 h |
|---|---|
| Cornea | 72.0 |
| Conjunctiva | 24.1 |
| Aqueous humor | 1.2 |
| Lens | 0.0 |
| Sclera | 0.0 |
| Iris | 0.0 |
| Choroid | 0.0 |
| Ciliary body | 0.0 |
| Vitreous | 0.0 |
| Retina | 0.0 |
| Lacrimal Gland | 0.0 |

### Alternative Gellan Gum-Based In-Situ Gel Formulation

Composition: 2.4-3% PS; 0.4% Gellan gum; 10% Vitamin E TPGS; 5% (2-hydroxypropyl)-β-cyclodextrin.

Preparation:

As above.

Results:

PS in this formulation was administered topically to the eyes of New Zealand white rabbits and its biodistribution was determined as above. Table 7 summarizes the findings.

TABLE 7

| | PS, μM | | |
|---|---|---|---|
| Time, h | Cornea | Conjunctiva | Aqueous humor |
| 0.5 | 24.3 | 37.7 | 0.6 |
| 1 | 50.8 | 20.8 | 0.4 |
| 3 | 1.5 | 0.7 | 0.0 |
| 5 | 1.1 | 1.1 | 0.0 |
| 8 | 1.6 | 0.7 | 0.0 |

### Sodium Alginate-Based In-Situ Gel Formulation

Composition: 3% PS, 1.5% sodium alginate, 5% Vitamin E TPGS, 10% (2-hydroxypropyl)β-cyclodextrin.

Preparation Method: A sodium alginate solution was prepared by adding a certain amount of sodium alginate to deionized water and heating the mixture to 90° C. with fast stirring (500 rpm). Once completely dissolved, the solution was filtered through a 0.22 μm filter. Then, PS and additional excipients were added to the system to achieve the above concentrations and stirred at 50° C. at 500 rpm for 30 minutes to allow complete dissolution.

### Alternative Sodium Alginate-Based In-Situ Gel Formulation

Composition:

3% PS, 1.5% sodium alginate, 15% Tween 80, 10% (2-hydroxypropyl)-β-cyclodextrin, 10% polyethylene glycol 400 (PEG400), 5% polyoxyl stearate.

Preparation Method:

A sodium alginate solution was prepared by adding an appropriate amount of sodium alginate to deionized water and heating the mixture to 90° C. with fast stirring (500 rpm). Once sodium alginate was completely dissolved, the solution was filtered through a 0.22 μm filter. Then, PS and additional excipients were added to achieve the above concentrations and stirred at 50° C. at 500 rpm until complete dissolution.

Results:

PS in this formulation was administered topically to the eyes of New Zealand white rabbits and its biodistribution was determined as above. Table 8 summarizes the findings.

TABLE 8

PS, μM

| Tissue | 1 h | 3 h | 5 h | 8 h |
|---|---|---|---|---|
| Cornea | 17.8 | 5.0 | 1.0 | 0.0 |
| Conjunctiva | 4.9 | 2.1 | 2.3 | 1.3 |
| Aqueous humor | 0.4 | 0.3 | 0.0 | 0.0 |
| Retina | 0.0 | 0.0 | 0.0 | 0.0 |

### Poloxamer 407-Based In-Situ Gel Formulation:

Composition:

5.4% PS; 20% Poloxamer 407; 12% Vitamin E TPGS.

Preparation Method:

Poloxamer 407 solution (thermosensitive gel solution) was prepared using a “cold method.” The required amount of Poloxamer 407 and other excipients were dissolved in cold double-distilled water at 4° C. The mixture was stirred continuously until a clear solution was obtained. Then the appropriate amount of PS was dissolved in cold PM solution with continuous stirring at room temperature until a clear solution formed.

Results:

PS in this formulation was administered topically as eye drops to the eyes of New Zealand white rabbits. The biodistribution of PS in ocular tissues at 3 h and 6 h was determined by HPLC. Table 9 summarizes the findings.

TABLE 9

PS, μM

| Tissue | 3 h | 6 h |
|---|---|---|
| Cornea | 45.1 | 13.6 |
| Conjunctiva | 5.6 | 10.7 |
| Aqueous humor | 0.3 | 0.3 |
| Iris | 0.0 | 0.0 |
| Lens | 0.0 | 0.0 |
| Vitreous | 0.0 | 0.0 |
| Retina | 0.0 | 0.0. |
| Choroid | 0.9 | 0.0 |
| Ciliary body | 0.0 | 0.0 |
| Sclera | 0.0 | 0.0 |

### Nanoparticle Formulation.

Composition:

˜3.0-3.5% PS, 96.5˜97% methoxy poly(ethylene glycol)-poly(lactide) (mPEG-PLA).

Preparation Method:

Oil phase: 150 mg of PS and 1 g of PEG-PLA (Akina, Inc) were dissolved in 20 mL dichloromethane (DCM). Water phase: 365 mg of sodium cholate were dissolved in 60 ml of purified water. 5 mL of the oil phase was gently added into 15 mL of the water phase in a 50 mL Eppendorf conical tube. To create an emulsion, we used robe sonication for 2 min at 75% output (Branson 150, Fisher Scientific™, USA); the watt output was 12-13. The emulsion was transferred into a 100 mL beaker and stirred overnight at 600 rpm in a chemical hood until the DCM was fully evaporated. This was followed by centrifugation at 14,000 rpm for 1 h (Dupont, RC-5C). Then, the supernatant was transferred to another tube into which 3 mL of PBS were added to resuspend the nanoparticles. The nanoparticle solution was centrifuged for 6-7 seconds to remove aggregates. This supernatant was the final preparation.

Results:

Characterization of PS Nanoparticles:

Effective diameter=109.4 nm; particle size distribution: polydispersity index=0.163; Drug Encapsulation Efficiency (EE)=46.4% (it was calculated as % EE=drug encapsulated/drug added *100).

Ocular PK Study:

PS formulated in nanoparticles as above was administered topically as eye drops to New Zealand white rabbits. The biodistribution of PS in ocular tissues at the indicated time points post administration was determined by HPLC. Tables 10 and 11 summarize these findings.

TABLE 10
PK Parameters of PS in rabbit eyes

| | | | AUC_{0-16 h}, |
|---|---|---|---|
| | Cₘₐₓ, μM | Tₘₐₓ, h | μM · h |
| Cornea | 101.3 | 1 | 156.5 |
| Conjunctiva | 26.7 | 1 | 61.7 |
| Aqueous humor | 2.6 | 1 | 3.4 |
| Iris | 5.2 | 1 | 13.6 |
| Lens | 0 | —* | 0.0 |
| Ciliary body | 2.6 | 1 | 3.6 |
| Vitreous body | 0 | — | 0.0 |
| Sclera | 2.1 | 1 | 3.3 |
| Choroid | 0 | — | 0.0 |
| Retina | 0 | — | 0.0 |

*cannot be calculated as PS was undetectable. Values are the average of two samples; in all cases the paired values were within <9%.)

Table 11: PK Parameters of PS and its metabolites in rabbit cornea and conjunctiva

| | | | | | |
|---|---|---|---|---|---|
| | | | | Sulin- | Sulin- |
| | PS | PS | Sulin- | dac | dac |
| PS | sulfide | sulfone | dac | sulfide | sulfone |

cornea

| | | | | | | |
|---|---|---|---|---|---|---|
| Cₘₐₓ, μM | 101.3 | 2.7 | 13 | 3.6 | 0.6 | 1.3 |
| Tₘₐₓ, h | 1 | 0.25 | 1 | 0.25 | 1 | 4 |
| AUC_{0-16 h}, | 156.5 | 3 | 56.9 | 19.1 | 0.7 | 10 |
| μM · h | | | | | | |

conjunctiva

| | | | | | | |
|---|---|---|---|---|---|---|
| Cₘₐₓ, μM | 26.7 | 0 | 4.7 | 3.8 | 0 | 1.6 |
| Tₘₐₓ, h | 1 | —* | 1 | 0.5 | — | 4 |
| AUC_{0-16 h}, | 61.7 | 0 | 7.2 | 6 | 0 | 7.3 |
| μM · h | | | | | | |

*cannot be calculated as PS was undetectable. Values are the average of two samples; in all cases the paired values were within <9%.

Biodistribution of PS after Intravitreal Injection:

PS formulated in nanoparticles as above was injected directly into the vitreous of New Zealand white rabbits. The biodistribution of PS in ocular tissues at the indicated time points post administration was determined by HPLC. Table 12 summarizes the findings.

TABLE 12

| | PS, μM | | | |
|---|---|---|---|---|
| | 2% PS | | 0.2% PS | |
| | Nanoparticle Soln. | | Nanoparticle Soln. | |
| Tissue | 0.5 h | 1 h | 0.5 h | 1 h |
| Cornea | 187.4 | 147.4 | 23.5 | 22.4 |
| Sclera | 223.7 | 180.2 | 39.8 | N.A. |
| Retina | 376.3 | 219.7 | 187.3 | 109.4 |
| Vitreous body | 125.4 | 34.0 | 198.5 | 56.2 |
| Aqueous humor | 0.0 | 1.3 | 0.0 | 0.1 |

Biodistribution of PS in Human Eyes (Ex Vivo):

Human cadaveric eyes were obtained through the Lions Eye Bank for Long Island, Valley Stream, N.Y. They were preserved on ice and used within 2 h from removal from the donors.

The anterior surface of the human eye (corresponding to an area slightly larger than the palpebral fissure) was brought into direct contact with a PS nanoparticle (NP) solution (PS concentrations were 0.2%, 1% and 2%) and treated as above for the solution formulations of PS. Table 13 summarizes the results.

TABLE 13

| | PS, μM | | |
|---|---|---|---|
| Tissue | 0.2% PS-NPs | 1% PS-NPs | 2% PS-NPs |
| Cornea | 22.8 | 58.8 | 92.7 |
| Iris | 8.0 | 35.5 | 17.4 |
| Lens | 0.4 | 1.6 | 0.6 |
| Retina | 2.2 | 4.8 | 1.2 |
| Sclera | 30.7 | 152.0 | 113.0 |

In another similar study, the anterior surface of the human eye was brought into direct contact with a PS HP-β-CD solution (PS concentration at 0.5%, 2.0% and 3.3%) and incubated for 10 min at 37° C. The eye was then rinsed with 10% dimethylsulfoxide (DMSO) to remove residual PS from the surface of the eye and incubated in PBS for 60 min. (Control experiments showed this DMSO concentration to completely remove PS without damaging the ocular tissues). At the specified times, ocular tissues were dissected and PS levels determined by HPLC. Table 14 summarizes the findings.

TABLE 14

| | PS, μM | | |
|---|---|---|---|
| Tissue | 3.3% PS | 2.0% PS | 0.5% PS |
| Cornea | 266.4 | 397.7 | 187.2 |
| Aqueous | 19.5 | ND | 2.4 |
| Iris | 169.3 | 34.2 | 25.6 |
| Lens | 1.9 | 1.4 | 0.6 |
| Vitreous | 4.3 | ND | 0.3 |
| Retina | 48.5 | 38.7 | 2.9 |
| Choroid | 261.4 | ND* | 28.5 |
| Sclera | 2,596.6 | 870.9 | 381.3 |

*ND: Not Determined

### Solution Formulations

One embodiment of this type of formulation of PS is the following: 2% PS, 16% Vitamin E TPGS, 3.18% mannitol, 1.2% boric acid, 0.005% polyquad (preservative). Preparation Method: Polyquaternium-1 and Vitamin E TPGS (D-α-Tocopheryl polyethylene glycol 1000 succinate) were dissolved in purified water, PS was added to this solution and stirred at 70° C. for 30 min. This solution was then centrifuged at 13,200 rpm for 10 min and the supernatant was collected. Mannitol and boric acid were added to the collected supernatant of the previous step. Purified water was then added to the final volume after pH adjustment to 6.7±0.2 using NaOH.

Another embodiment of this type of formulation is: 0.1% PS, 10% HP-β-CD, 4% Tween 80, 2.5% Vitamin E TPGS, 1.4% polyvinyl alcohol (PVA) (13,000-26,000 molecular weight), 0.001% polyquad. Another embodiment of this type of formulation is: 0.2% PS; 10% HP-β-CD; 4% Tween 80; 2.5% Vitamin E TPGS; 1.4% polyvinyl alcohol (PVA) (13,000-26,000 molecular weight); and 0.001% polyquad. Preparation method: PVA was dissolved into water by stirring at 95° C. for 6 h. All ingredients including the PVA solution and PS were added into a glass vial, stirred at 50° C. (in a water bath) for 4 h, and then stirred at RT overnight. The pH was adjusted to 7.4±0.2 with NaOH and the osmolarity to 280-320 mOsm with NaCl 18%. For a sterile final product, this solution was filtered through a 0.22 μM film.

The corneal levels of a PS 0.2% formulation were determined after its single topical application to the surface of the eye. The following results were obtained:

| | Cornea |
|---|---|
| Parameter | mean ± SEM |
| Cₘₐₓ, μM | 63.4 ± 10.0 |
| tₘₐₓ, h | 0.5 ± 0.0 |
| t_{1/2}, h | 0.7 ± 0.2 |
| AUC ₀₋ₜ, μM · h | 62.6 ± 10.2 |
| MRT _{0-inf}_obs, h | 1.0 ± 0.1 |

Other Solution Formulations

PS=0.1-1.3% (w/v); HP-β-CD=10% (w/v); Tween 80 (v/v)=4% (range: 0-20%); Vitamin E TPGS (w/v)=2.5%; polyvinyl alcohol (PVA) (13000-23000 molecular weight) 0-1.4% (w/v); carboxymethylcellulose (low, medium and high viscosity) 0-0.5% (w/v); polyquad (Polyquaternium-1)=0.001% (w/v). Preparation method: When PVA was included in the formulation, it was dissolved first into water by stirring at 95° C. for 6 h. When CMC was included in the formulation, it was dissolved into water it was heated at 50° C. for 2 h or until completely dissolved. When both PVA and CMC were used together, solutions of the two were made independently and maintained at room temperature (RT). After that, all ingredients including the PVA and or CMC solution(s) and PS were added into a glass vial, stirred at 50° C. (in a water bath) for 4 h, and then stirred at RT overnight. The pH was adjusted to 7.4±0.2 with NaOH and the osmolarity to 280-320 mOsm with NaCl 18%. For a sterile final product, this solution was filtered through a 0.22 μM film.

A formulation is made as following: PS=0.1% (w/v); HP-β-CD=10% (w/v); Tween 80 (v/v)=4%; Vitamin E TPGS (w/v)=2.5%; polyvinyl alcohol (PVA) (13,000-26,000 molecular weight) 1.4% (w/v); carboxymethylcellulose (medium viscosity) 0.5%; polyquad (Polyquaternium-1)=0.001% (w/v).

Another formulation is made as following: PS=0.1% (w/v); HP-β-CD=10% (w/v); Tween 80 (v/v)=4%; Vitamin E TPGS (w/v)=2.5%; carboxymethylcellulose (medium viscosity) 0.5% (w/v); polyquad (Polyquaternium-1)=0.001% (w/v).

Another formulation is made as following: PS=0.1% (w/v); HP-β-CD=10% (w/v); Tween 80 (v/v)=4%; Vitamin E TPGS (w/v)=2.5%; polyvinyl alcohol (PVA) (13,000-26,000 molecular weight) 1.4% (w/v); polyquad (Polyquaternium-1)=0.001% (w/v).

PS Solution Formulation Development for Ocular Application

PS 1.6%—Process: Dissolve PVA (MW, 13000-23000) into water by stirring at 95° C. for 6 h. Add all ingredients into glass vial including PS, stir at 50° C. (water bath) for 4 h, stir at RT overnight. Adjust pH and Osmolarity. Optionally, for a sterile solution, filter through 0.22 μM film.

| Ingredient | Composition, % | Amount | |
|---|---|---|---|
| PS | 1.6 | 16 | mg |
| HP-B-CD | 10 | 100 | mg (powder) |
| VETPGS | 2.5 | 250 μL of 10% solution | |
| | | in H₂O | |
| Tween 80 | 4 | 40 | μL (liquid) |
| PVA | 1.4 | 280 μL of 5% solution | |
| | | in H₂O | |
| Polyquaternium-1 | 0.001 | 2 μL of 0.5% aqueous | |
| | | solution | |
| 18% NaCl | Adjust Osmolarity | ~20 | μL |
| | to 280-320 mosm/kg | | |
| NaOH 2M | Adjust pH to | ~5 | μL |
| | 7.4 ± 0.2 | | |
| Water | | Up to 1000 μL | |
| Total | 100 | 1 | ml |

PS 0.1%—Process: Dissolve PVA (MW, 13000-23000) into water by stirring at 95° C. for 6 h. Add all ingredients into glass vial including PS, stir at 50° C. (water bath) for 4 h, stir at RT overnight. Adjust pH and Osmolarity, and then optionally filtrate through 0.22 μM film to get the final product sterile.

| Ingredient | Composition, % | Amount | |
|---|---|---|---|
| PS | 0.1 | 1 | mg |
| HP-B-CD | 10 | 100 | mg (powder) |
| VETPGS | 2.5 | 250 μL of 10% solution | |
| | | in H₂O | |
| Tween 80 | 4 | 40 | μL (liquid) |
| PVA | 1.4 | 280 μL of 5% solution | |
| | | in H₂O | |
| Polyquaternium-1 | 0.001 | 2 μL of 0.5% aqueous | |
| | | solution | |
| 18% NaCl | Adjust Osmolarity | ~20 | μL |
| | to 280-320 mosm/kg | | |
| NaOH 2M | Adjust pH to | ~5 | μL |
| | 7.4 ± 0.2 | | |
| Water | | Up to 1000 μL | |
| Total | 100 | 1 | ml |

PS 0.1% with CMC no PVA—Process: Dissolve CMC Na (medium viscosity) into water by stirring at 50° C. for 1 h. Add all ingredients into glass vial including PS, stir at 50° C. (water bath) for 4 h, stir at RT overnight.

| Ingredient | Composition, % | Amount |
|---|---|---|
| PS | 0.1 | 10 mg |
| HP-B-CD | 10 | 1000 mg (takes about 0.5 ml |
| | | volume) |
| VETPGS | 2.5 | 2.5 ml of 10% aq. solution |
| Tween 80 | 4 | 0.4 ml |
| CMC Na (medium | 0.5 | 3.3 ml 1.5% aq. solution |
| viscosity) | | |
| Polyquaternium-1 | 0.001% | 20 μL 0.5% solution |
| 18% NaCl | Adjust Osmolarity | |
| | to 280-320 mosm/kg | |
| NaOH 2M | Adjust pH to | |
| | 7.4 ± 0.2 | |
| Water | Up to 100 | 3.1 ml (including pH |
| | | adjustment) |
| Total | 100 | 10 mL |

PS 0.1% with CMC and PVA—Process: Add PVA solution into water by stirring at 50° C. for 1 h. Add all ingredients into glass vial including PS, stir at 50° C. (water bath) for 4 h, stir at RT overnight.

| Ingredient | Composition, % | Amount | |
|---|---|---|---|
| PS | 0.1 | 1 | mg |
| HP-B-CD | 10 | 100 | mg (powder) |
| VETPGS | 2.5 | 250 μL of 10% solution | |
| | | in H₂O | |
| Tween 80 | 4 | 40 | μL (liquid) |
| PVA | 1.4 | 280 μL of 5% solution | |
| | | in H₂O | |
| CMC Na (medium | 0.5 | 5 | mg (powder) |
| viscosity) | | | |
| Polyquaternium-1 | 0.001 | 2 μL of 0.5% aqueous | |
| | | solution | |
| 18% NaCl | Adjust Osmolarity | ~20 | μL |
| | to 280-320 mosm/kg | | |
| NaOH 2M | Adjust pH to | ~5 | μL |
| | 7.4 ± 0.2 | | |
| Water | | Up to 1000 μL | |
| Total | 100 | 1 | ml |

Hydrogel Formulations

PS was formulated in hydrogels, with two exemplary formulations described herein. Hydrogel formulation containing PS 0.2%: 0.2% PS, 4% HP-β-CD, 0.6% Tween 80, 0.45% Carbopol 980, 0.2% Vitamin E TPGS, 0.3% PVA (13,000-26,000 molecular weight), NaCl and mannitol (isotonic reagent). Preparation method: Dissolved Carbopol 980 into water at concentration of 0.6%, adjust pH to 6.0 to form a gel. Prepared stock solution of PS at 0.8% concentration. Mixed 1 ml 0.8% PS stock solution with 3 ml prepared Carbopol gel and vortexed to obtain the PS hydrogel.

| | |
|---|---|
| Ingredient | Composition, % |

Stock Solution

| | |
|---|---|
| PS | 0.8 |
| HP-β-CD | 16 |
| VETPGS | 0.8 |
| Mannitol | 4.2 |
| NaCl | 0.3 |
| Tween 80 | 2.4 |
| PVA (13000-23000 MW) | 1.12 |
| Water | Up to 100 |
| Total | 100 |

Final Concentrations

| | |
|---|---|
| PS | 0.2 |
| HP-B-CD | 4 |
| VETPGS | 0.2 |
| mannitol | 1.3 |
| NaCl | 0.075 |
| Tween 80 | 0.6 |
| PVA (13000-23000 MWt) | 0.28 |
| Carbopol 980 | 0.45 |
| Water | 92.9 |
| Total | 100 |

Hydrogel formulation containing PS 0.6%: 0.6% PS, 5% HP-β-CD, 4% Tween 80, 0.45% Carbopol 980, 1.25% Vitamin E TPGS, 0.8% PVA (13,000-26,000 molecular weight), mannitol (isotonic reagent). Preparation method: Carbopol 980 was dissolved into water at concentration of 0.9%, pH was adjusted to 6.0 to form a gel. Stock solution of PS was prepared as follows. Added 2 ml 1.2% PS stock solution to 2 ml prepared Carbopol gel and vortexed to obtain the PS hydrogel.

Ointment Formulation

PS was formulated as an ointment. Composition: 1% PS, 5% propylene glycol (PG), 5% Tween 60, 30% mineral oil, 59% petrolatum. Preparation method: PS was dissolved in PG by stirring at 50° C., mineral oil and Tween 60 were added, and the mixture kept at 50° C. Petrolatum was preheated to 50° C. to allow its complete melting, and added to the PS solution. The resultant solution was mixed well and cooled down to room temperature to obtain a uniform PS ointment.

Formulations Containing Terpenes or their Derivatives

Terpenes and their derivatives such as menthol were used in ocular formulations of PS because of their cooling and analgesic properties. In exemplary formulations of PS containing menthol solution formulations as those described above were used and menthol was added at a concentration that ranged between 0.025 and 0.1%.

These findings indicate, without being limited to any one theory of the invention, that each of the various formulations exemplified herein targets PS to ocular tissues in a specific manner.

### REFERENCES

1. The definition and classification of dry eye disease: report of the Definition and Classification Subcommittee of the International Dry Eye WorkShop (2007). Ocul Surf 2007; 5(2):75-92.
2. Phadatare S P, Momin M, Nighojkar P, Askarkar S, Singh K K. A Comprehensive Review on Dry Eye Disease: Diagnosis, Medical Management, Recent Developments, and Future Challenges. Advances in Pharmaceutics 2015; 2015:1-12.
3. Paulsen A J, Cruickshanks K J, Fischer M E, Huang G H, Klein B E, Klein R, et al. Dry eye in the beaver dam offspring study: prevalence, risk factors, and health-related quality of life. Am J Ophthalmol 2014; 157(4):799-806.
4. The epidemiology of dry eye disease: report of the Epidemiology Subcommittee of the International Dry Eye WorkShop (2007). Ocul Surf 2007; 5(2):93-107.
5. Lin H, Yiu S C. Dry eye disease: A review of diagnostic approaches and treatments. Saudi J Ophthalmol 2014; 28(3):173-81.
6. de Paiva C S, Pflugfelder S C. Rationale for anti-inflammatory therapy in dry eye syndrome. Arq Bras Oftalmol 2008; 71(6 Suppl):89-95.
7. Hessen M, Akpek E K. Dry eye: an inflammatory ocular disease. J Ophthalmic Vis Res 2014; 9(2):240-50.
8. Lan W, Petznick A, Heryati S, Rifada M, Tong L. Nuclear Factor-kappaB: central regulator in ocular surface inflammation and diseases. Ocul Surf 2012; 10(3):137-48.
9. Peng W J, Yan J W, Wan Y N, Wang B X, Tao J H, Yang G J, et al. Matrix metalloproteinases: a review of their structure and role in systemic sclerosis. J Clin Immunol 2012; 32(6):1409-14.
10. Yoon K C, De Paiva C S, Qi H, Chen Z, Farley W J, Li D Q, et al. Expression of Th-1 chemokines and chemokine receptors on the ocular surface of C57B L/6 mice: effects of desiccating stress. Invest Ophthalmol Vis Sci 2007; 48(6):2561-9.
11. The management of dry eye. BMJ 2016; 354:i4463.
12. Moshirfar M, Pierson K, Hanamaikai K, Santiago-Caban L, Muthappan V, Passi S F. Artificial tears potpourri: a literature review. Clin Ophthalmol 2014; 8:1419-33.
13. Wan K H, Chen L J, Young A L. Efficacy and Safety of Topical 0.05% Cyclosporine Eye Drops in the Treatment of Dry Eye Syndrome: A Systematic Review and Meta-analysis. Ocul Surf 2015; 13(3):213-25.
14. Zhou X Q, Wei R L. Topical cyclosporine A in the treatment of dry eye: a systematic review and meta-analysis. Cornea 2014; 33(7):760-7.
15. Perez V L, Pflugfelder S C, Zhang S, Shoj aei A, Haque R. Lifitegrast, a Novel Integrin Antagonist for Treatment of Dry Eye Disease. Ocul Surf 2016; 14(2):207-15.
16. Semba C P, Gadek T R. Development of lifitegrast: a novel T-cell inhibitor for the treatment of dry eye disease. Clin Ophthalmol 2016; 10:1083-94.
17. Gaynes B I, Onyekwuluje A. Topical ophthalmic NSAIDs: a discussion with focus on nepafenac ophthalmic suspension. Clin Ophthalmol 2008; 2(2):355-68.
18. Mackenzie G G, Sun Y, Huang L, Xie G, Ouyang N, Gupta R C, et al. Phospho-sulindac (OXT-328), a novel sulindac derivative, is safe and effective in colon cancer prevention in mice. Gastroenterology 2010; 139(4): 1320-32.
19. Cheng K W, Wong C C, Alston N, Mackenzie G G, Huang L, Ouyang N, et al. Aerosol administration of phospho-sulindac inhibits lung tumorigenesis. Mol Cancer Ther 2013; 12(8):1417-28.
20. Huang L, Mackenzie G, Ouyang N, Sun Y, Xie G, Johnson F, et al. The novel phospho-non-steroidal anti-inflammatory drugs, OXT-328, MDC-22 and MDC-917, inhibit adjuvant-induced arthritis in rats. Br J Pharmacol 2011; 162(7):1521-33.
21. Wong C C, Cheng K W, Papayannis I, Mattheolabakis G, Huang L, Xie G, et al. Phospho-NSAIDs have enhanced efficacy in mice lacking plasma carboxylesterase: implications for their clinical pharmacology. Pharmaceutical research 2015; 32(5):1663-75.
22. Wong C C, Cheng K W, Xie G, Zhou D, Zhu C H, Constantinides P P, et al. Carboxylesterases 1 and 2 hydrolyze phospho-nonsteroidal anti-inflammatory drugs: relevance to their pharmacological activity. J Pharmacol Exp Ther 2012; 340(2):422-32.
23. Schrader S, Mircheff A K, Geerling G. Animal models of dry eye. Dev Ophthalmol 2008; 41:298-312.
24. Xiong C, Chen D, Liu J, Liu B, Li N, Zhou Y, et al. A rabbit dry eye model induced by topical medication of a preservative benzalkonium chloride. Invest Ophthalmol Vis Sci 2008; 49(5):1850-6.
25. Barabino S. Animal models of dry eye. Arch Soc Esp Oftalmol 2005; 80(12):693-4; 95-6.
26. Barabino S, Chen W, Dana M R. Tear film and ocular surface tests in animal models of dry eye: uses and limitations. Exp Eye Res 2004; 79(5):613-21.
27. Barabino S, Dana M R. Animal models of dry eye: a critical assessment of opportunities and limitations. Invest Ophthalmol Vis Sci 2004; 45(6):1641-6.
28. Singh S, Moksha L, Sharma N, Titiyal J S, Biswas N R, Velpandian T. Development and evaluation of animal models for sex steroid deficient dry eye. J Pharmacol Toxicol Methods 2014; 70(1):29-34.
29. Burgalassi S, Panichi L, Chetoni P, Saettone M F, Boldrini E. Development of a simple dry eye model in the albino rabbit and evaluation of some tear substitutes. Ophthalmic research 1999; 31(3):229-35.
30. Nagelhout T J, Gamache D A, Roberts L, Brady M T, Yanni J M. Preservation of tear film integrity and inhibition of corneal injury by dexamethasone in a rabbit model of lacrimal gland inflammation-induced dry eye. J Ocul Pharmacol Ther 2005; 21(2):139-48.
31. Seo M J, Kim J M, Lee M J, Sohn Y S, Kang K K, Yoo M. The therapeutic effect of DA-6034 on ocular inflammation via suppression of MMP-9 and inflammatory cytokines and activation of the MAPK signaling pathway in an experimental dry eye model. Curr Eye Res 2010; 35(2):165-75.
32. Zheng W, Ma M, Du E, Zhang Z, Jiang K, Gu Q, et al. Therapeutic efficacy of fibroblast growth factor 10 in a rabbit model of dry eye. Mol Med Rep 2015; 12(5):7344-50.
33. Williams J L, Ji P, Ouyang N, Liu X, Rigas B. N O-donating aspirin inhibits the activation of N F-kappaB in human cancer cell lines and Min mice. Carcinogenesis 2008; 29(2):390-7.
34. Davis F A. The Anatomy and Histology of the Eye and Orbit of the Rabbit. Trans Am

Ophthalmol Soc 1929; 27:400 2-41.
35. Senchyna M, Wax M B. Quantitative assessment of tear production: A review of methods and utility in dry eye drug discovery. J Ocul Biol Dis Infor 2008; 1(1):1-6.
36. Demetriades A M, Leyngold I M, D'Anna S, Eghrari A O, Emmert D G, Grant M P, et al. Intraglandular injection of botulinum toxin a reduces tear production in rabbits. Ophthal Plast Reconstr Surg 2013; 29(1):21-4.
37. Enriquez-de-Salamanca A, Castellanos E, Stern M E, Fernandez I, Carreno E, Garcia-Vazquez C, et al. Tear cytokine and chemokine analysis and clinical correlations in evaporative-type dry eye disease. Mol Vis 2010; 16:862-73.
38. Cargnello M, Roux P P. Activation and function of the MAPKs and their substrates, the MAPK-activated protein kinases. Microbiol Mol Biol Rev 2011; 75(1):50-83.
39. Pflugfelder S C, Wilhelmus K R, Osato M S, Matoba A Y, Font R L. The autoimmune nature of aqueous tear deficiency. Ophthalmology 1986; 93(12):1513-7.
40. Luo L, Li D Q, Doshi A, Farley W, Corrales R M, Pflugfelder S C. Experimental dry eye stimulates production of inflammatory cytokines and MMP-9 and activates MAPK signaling pathways on the ocular surface. Invest Ophthalmol Vis Sci 2004; 45(12):4293-301.
41. Leonardi A, Brun P, Abatangelo G, Plebani M, Secchi A G. Tear levels and activity of matrix metalloproteinase (MMP)-1 and MMP-9 in vernal keratoconjunctivitis. Invest Ophthalmol Vis Sci 2003; 44(7):3052-8.
42. Sobrin L, Liu Z, Monroy D C, Solomon A, Selzer M G, Lokeshwar B L, et al. Regulation of MMP-9 activity in human tear fluid and corneal epithelial culture supernatant. Invest Ophthalmol Vis Sci 2000; 41(7):1703-9.
43. Pflugfelder S C, Farley W, Luo L, Chen L Z, de Paiva C S, Olmos L C, et al. Matrix metalloproteinase-9 knockout confers resistance to corneal epithelial barrier disruption in experimental dry eye. Am J Pathol 2005; 166(1):61-71.
44. Kim H S, Luo L, Pflugfelder S C, Li D Q. Doxycycline inhibits TGF-beta1-induced MMP-9 via Smad and MAPK pathways in human corneal epithelial cells. Invest Ophthalmol Vis Sci 2005; 46(3):840-8.
45. Solomon A, Dursun D, Liu Z, Xie Y, Macri A, Pflugfelder S C. Pro- and anti-inflammatory forms of interleukin-1 in the tear fluid and conjunctiva of patients with dry-eye disease. Invest Ophthalmol Vis Sci 2001; 42(10):2283-92.
46. Li D Q, Luo L, Chen Z, Kim H S, Song X J, Pflugfelder S C. JNK and ERK MAP kinases mediate induction of IL-1beta, TNF-alpha and IL-8 following hyperosmolar stress in human limbal epithelial cells. Exp Eye Res 2006; 82(4):588-96.
47. Pflugfelder S C, Jones D, Ji Z, Afonso A, Monroy D. Altered cytokine balance in the tear fluid and conjunctiva of patients with Sjögren's syndrome keratoconjunctivitis sicca. Curr Eye Res 1999; 19(3):201-11.
48. Abelson MBL, Lauren. Melting Away the Myths of NSAIDs. Review of Ophthalmology 2007; 14(11): 124-28.
49. Shim J, Park C, Lee H S, Park M S, Lim H T, Chauhan S, et al. Change in prostaglandin expression levels and synthesizing activities in dry eye disease. Ophthalmology 2012; 119(11):2211-9.
50. McGinnigle S, Naroo S A, Eperjesi F. Evaluation of dry eye. Sury Ophthalmol 2012; 57(4):293-316.
51. Mackenzie G G, Ouyang N, Xie G, Vrankova K, Huang L, Sun Y, et al. Phospho-sulindac (OXT-328) combined with difluoromethylornithine prevents colon cancer in mice. Cancer Prev Res (Phila) 2011; 4(7):1052-60.
52. Guidera A C, Luchs J I, Udell U. Keratitis, ulceration, and perforation associated with topical nonsteroidal anti-inflammatory drugs. Ophthalmology 2001; 108(5):936-44.
53. Galor A, Feuer W, Lee D J, Florez H, Venincasa V D, Perez V L. Ocular surface parameters in older male veterans. Invest Ophthalmol Vis Sci 2013; 54(2):1426-33.
54. Satitpitakul V, Kheirkhah A, Crnej A, Hamrah P, Dana R. Determinants of Ocular Pain Severity in Patients With Dry Eye Disease. Am J Ophthalmol 2017; 179:198-204.

1-86. (canceled)

87. A pharmaceutical composition comprising a compound selected from a compound of formula I and a compound of formula II: 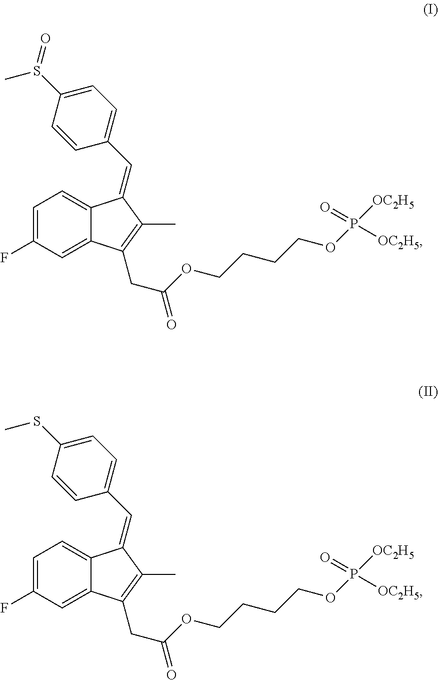 [SMILES: CCOP(=O)(OCC)OCCCCOC(=O)CC1=C(C)/C(=C/c2ccc(S(C)=O)cc2)c2ccc(F)cc21 ; CCOP(=O)(OCC)OCCCCOC(=O)CC1=C(C)/C(=C/c2ccc(SC)cc2)c2ccc(F)cc21]
or a pharmaceutically acceptable salt thereof.

88. The composition of claim 87, wherein the composition comprises 20% or less by weight of the compound of formula I or formula II, or the pharmaceutically acceptable salt thereof.

89. The composition of claim 87, wherein the composition comprises about 0.20% or less by weight of the compound of formula I or formula II, or the pharmaceutically acceptable salt thereof.

90. The composition of claim 87, wherein the composition comprises about 0.10% or less by weight of the compound of formula I or formula II, or the pharmaceutically acceptable salt thereof.

91. The composition of claim 87, further comprising one or more of a cyclodextrin, a solubilizing agent, a surfactant, a polyvinyl alcohol (PVA), Carbopol® 980, a sugar alcohol, and a preservative.

92. The composition of claim 91, wherein the cyclodextrin comprises (2-hydroxypropyl)-β-cyclodextrin (HP-β-CD).

93. The composition of claim 92, wherein the composition comprises between about 0% and about 20% HP-β-CD by weight.

94. The composition of claim 92, wherein the composition comprises about 5% HP-β-CD by weight.

95. The composition of claim 91, wherein the solubilizing agent comprises vitamin E TPGS (d-α-tocopheryl polyethylene glycol 1000 succinate).

96. The composition of claim 95, wherein the composition comprises less than about 25% vitamin E TPGS by weight.

97. The composition of claim 95, wherein the composition comprises about 1.25% vitamin E TPGS by weight.

98. The composition of claim 91, wherein the surfactant comprises Tween 80.

99. The composition of claim 98, wherein the composition comprises between 0% and about 25% Tween 80 by volume.

100. The composition of claim 99, wherein the composition comprises about 2% Tween 80 by volume.

101. The composition of claim 91, wherein the PVA has a molecular weight of 13,000-26,000.

102. The composition of claim 91, wherein the composition comprises between about 0% and about 2.5% PVA by weight.

103. The composition of claim 91, wherein the composition comprises about 0.7% PVA by weight.

104. The composition of claim 91, wherein the composition comprises between about 0.1% and about 1.5% Carbopol® 980 by weight.

105. The composition of claim 91, wherein the composition comprises about 0.45% Carbopol® 980 by weight.

106. The composition of claim 91, wherein the sugar alcohol is mannitol.

107. The composition of claim 106, wherein the composition comprises between 0% and about 10% mannitol by weight.

108. The composition of claim 106, wherein the composition comprises about 2% mannitol by weight.

109. The composition of claim 91, wherein the preservative is polyquaternium-1 (polyquad).

110. The composition of claim 109, wherein the composition comprises between 0% and about 5% polyquad by weight.

111. The composition of claim 109, wherein the composition comprises about 0.001% polyquad by weight.

112. The composition of claim 87, further comprising a therapeutically effective amount of an additional active agent.

113. The composition of claim 112, wherein the additional active agent is selected from the group consisting of an antibiotic, cyclosporine, lifitegrast, and a combination thereof.

114. A method of treating an ophthalmic condition in a patient in need thereof, the method comprising administering to the patient a therapeutically effective amount of the pharmaceutical composition according to claim 87.

115. The method of claim 114, wherein the ophthalmic condition is one or more of dry eye disease, retinopathy, uveitis, ocular pain, ocular discomfort, ocular burning sensation, ocular sensation of dryness in the eye, or inflammation in the eye.

116. The method of claim 115, wherein the retinopathy is selected from the group consisting of diabetic retinopathy, retinopathy of prematurity, VEGF retinopathy, age related macular degeneration, retinal vein occlusion, and hypertensive retinopathy.

117. The method of claim 114, wherein the composition is delivered to a site of action via one or more of: an oral route, an intraduodenal route, parenteral injection, intravenous injection, intraarterial injection, subcutaneous injection, intramuscular injection, intravascular injection, intraperitoneal injection, infusion, topical application, transdermal application, ocular application, rectal administration, local delivery by a catheter, local delivery by an implantable slow-release device, local delivery by a stent, inhalation, intraadiposally, intrathecally, and intraocular injection.

118. The method of claim 117, wherein the topical application is via an eye drop.

119. A method of relieving pain in a patient in need thereof, the method comprising administering to the patient a therapeutically effective amount of the pharmaceutical composition according to claim 87.
